(19) United States
(12) Patent Application Publication
(10) Pub. No.: US 2026/0327572 A1
(43) Pub. Date: Oct. 22, 2026

(54) TRANSFECTION OF CELLS VIA REVERSIBLE PERMEABILIZATION

(71) Applicant: Avectas Limited, Co. Dublin (IE)

(72) Inventors: Darren Martin, Co. Kildare (IE); Emer Hackett, Dublin (IE)

(21) Appl. No.: 19/526,660

(22) PCT Filed: Oct. 9, 2024
(86) PCT No.: PCT/IB2024/059886

§ 371 Date: Apr. 8, 2026

Related U.S. Application Data
(60) Provisional application No. 63/588,910, filed on Oct. 9, 2023.
(60) Provisional application No. 63/620,207, filed on Jan. 12, 2024.

(51) Int. Cl. C12N 5/074; C12N 15/87

(52) U.S. Cl. CPC C12N 5/0696; C12N 15/87

(57) ABSTRACT

The current subject matter provides a cell engineering platform for vector-free and/or viral delivery of payload/cargo compounds and compositions into cells. The platform achieves delivery to cells quickly and in an easy to use manner in an increased environmental temperature. Also provided herein are non-viral methods for reprogramming blood cells to induced pluripotent stem cells (iPSCs). Related apparatus, systems, techniques, articles and compositions are also described.

### RELATED APPLICATIONS

This application claims the benefit of priority under 35 U.S.C. § 119(e) to U.S. Provisional Application No. 63/588,910, filed Oct. 9, 2023, and U.S. Provisional Application No. 63/620,207, filed Jan. 12, 2024, the entire contents of each of which is incorporated herein by reference in their entireties.

### TECHNICAL FIELD

The subject matter described herein relates to a cell engineering platform utilizing solution-based intracellular delivery.

### BACKGROUND

Variability in intracellular delivery efficiency exists among different cell types and intracellular delivery methods. Obtaining sufficient quantities of viable cells following intracellular delivery can require large scale cell engineering platforms, which can be costly to operate and require of greater quantities of target cells and reagent media. Rapidly generating high-quality, repeatable experimental data from reversibly permeabilizing smaller quantities of target cells can be time-consuming and manually intensive using conventional systems and methods.

### SUMMARY

In an aspect, a method includes filling a pod of a cell engineering platform with a mixture of cells and a first medium; and discharging the first medium from the pod through a filter, leaving the cells deposited on the filter. The cell engineering platform includes an atomizer; and a pod holder configured to receive the pod. The pod includes a filter plate and an upper portion forming a well for holding cells and media.

One or more of the following features can be included in any feasible combination.

In aspects, provided herein are methods for delivering a payload across a plasma membrane of an adherent cell, the method including, providing a population of adherent cells, contacting the population of cells with a volume of an aqueous solution, the aqueous solution including the payload and an alcohol at greater than 2 percent (v/v) concentration, and wherein the contacting the population of cells with the volume of the aqueous solution is performed in an environment having a temperature greater than 25 °C.

In some embodiments, the adherent cell include stem cells, wherein the stem cells comprise induced pluripotent stem cells (iPSCs), primary mesenchymal stem cells, neuronal stem cells, hematopoietic stem cells, mouse embryonic stem cells, and human embryonic stem cells.

In other embodiments, the environment has a temperature greater than 25 °C, or wherein the environment has a temperature of about 37 °C, or wherein the environment has a temperature from about 37 °C to about 25 °C. In some embodiments, the population of cells has a temperature of greater than 25 °C, or of about 37 °C during the contacting step. In embodiments, the aqueous solution has a temperature of greater than 25 °C, or of about 37 °C during the contacting step. In embodiments, the population of cells and the aqueous solution have a temperature the same as the environment. In still other embodiments, the environment is an environment enclosed within an incubator. In embodiments, the stop solution has a temperature of greater than 25 °C, or of about 37 °C during the contacting step.

In aspects, provided herein is a system having an incubator including an internal environment and configurable to maintain a temperature of the internal environment at greater than 25 °C; and a delivery platform configurable to deliver a payload to a population of cells, the delivery platform comprising: a pod including a filter plate and an upper portion forming a well; a housing including a pod holder configured to receive the pod; a delivery solution applicator configured to deliver atomized delivery solution to the well; a display; and a controller including circuitry configured to display at least one process parameter, wherein the delivery platform is located within the internal environment of the incubator.

In embodiments, the incubator has a temperature greater than 25 °C, or wherein the incubator has a temperature of about 37 °C, or wherein the incubator has a temperature from about 37 °C to about 25 °C.

In aspects, provided herein is a system having an incubator including an internal environment and configurable to maintain a temperature of the internal environment at greater than 25 °C; and a device for use to deliver a cargo to cells in the absence of alcohol, the device having a housing including a base, at least one controller including circuitry configured to control an operation of the device, and a display; one or more fluid circuits including at least one valve, at least one pump, a syringe, and at least one fluid detection sensor; a chamber assembly received within an articulating frame extending from the front surface of the housing, wherein the chamber assembly is sealed from atmospheric conditions in operation and includes a filter; at least one media container; at least one cell culture container fluidically coupled to the chamber assembly via the one or more fluid circuits; and at least one collection tray configured to receive media or cells, wherein the delivery platform is located within the internal environment of the incubator.

In embodiments, the incubator has a temperature greater than 25 °C, or wherein the incubator has a temperature of about 37 °C, or wherein the incubator has a temperature from about 37 °C to about 25 °C.

In embodiments, provided herein is a method of reprogramming a population of blood cells to a population of induced pluripotent stem cells (iPSCs), where the method includes providing a population of blood cells, by at least transfecting the population of blood cells, and contacting the population of blood cells with a volume of an aqueous solution, the aqueous solution including a nucleic acid molecule and an alcohol at a greater than 2 percent (v/v) concentration, thereby reprogramming the population of blood cells to a population of iPSCs.

In aspects, the method of reprogramming includes blood cells including CD34+ cells or peripheral blood mononuclear cells (PBMCs).

In aspects, the population of blood cells is at a density of about 5e4 to about 5e7. In aspects, the population of blood cells is at a density of about 5e4, or about 5e5, or about 5e6, or about 5e7. In aspects, the population of blood cells is at a density of about 5e6 to about 5e7, or from about 5e5 to about 5e6.

In aspects, the nucleic acid molecule comprises a ribonucleic acid (RNA) molecule. In further aspects, the RNA molecule is self-replicating RNA (srRNA), messenger RNA (mRNA), siRNA, or RNAi.

In certain aspects, the population of blood cells is transfected between 2 and 10 times. In other aspects, the population of blood cells is transfected 5 times, or 6 times, or 7 times, or 8 times, or 9 times, or 10 times. In aspects, the population of blood cells is transfected 7 times. In aspects, the population of blood cells is transfected 8 times.

In aspects, the method includes performing multiple transfections on the population of blood cells.

In aspects, the aqueous solution includes an isotonic aqueous solution. For example, the solution is isotonic with respect to the cytoplasm of a mammalian cell such as a human T cell. Such an exemplary isotonic delivery solution 106 mM KCl (potassium chloride). The aqueous solution can include an ethanol concentration of 5 to 30%. The aqueous solution can include one or more of 75 to 98% H₂O, 2 to 45% ethanol, 6 to 91 mM sucrose, 2 to 500 mM KCl, 2 to 35 mM ammonium acetate, and 1 to 14 mM (4-(2-hydroxyethyl)-1-piperazineethanesulfonic acid) (HEPES).

In other aspects, the nucleic acid molecule encodes a gene-editing composition.

In embodiments, provided herein is a population of blood-cell derived induced pluripotent stem cells (iPSCs), where the population is generated without the use of a virus or viral vector. In aspects, the blood cells are contacted with a volume of an aqueous solution, the aqueous solution including a nucleic acid molecule and an alcohol at a greater than 2 percent (v/v) concentration.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is an isometric view of a computer aided design (CAD) drawing illustrating an example embodiment of a delivery platform according to some embodiments disclosed herein.

FIG. 2A is a side view of the delivery platform shown in FIG. 1.

FIG. 2B is a front view of the delivery platform shown in FIG. 1.

FIG. 3 is a side view of another example embodiment of the delivery platform shown in FIG. 1, according to some embodiments discloses herein.

FIG. 4A is an isometric view of a CAD drawing illustrating an example embodiment of a base assembly of the delivery platform shown in FIG. 1.

FIG. 4B is a pneumatic diagram of some implementations of the platform shown in FIG. 1.

FIG. 5 is an isometric view of a CAD drawing illustrating an example embodiment of a spine assembly of the delivery platform shown in FIG. 1.

FIG. 6A-B is an isometric view of a CAD drawing illustrating an example embodiment of a top assembly of the delivery platform shown in FIG. 1.

FIGS. 7A-7F are CAD drawings illustrating an example Eppendorf base support of the delivery platform of FIG. 1.

FIGS. 8A-8E are CAD drawings illustrating an example upper mount of a clippard module of the delivery platform of FIG. 1.

FIGS. 9A-9H are CAD drawings illustrating an example lower mount of a clippard module of the delivery platform of FIG. 1.

FIG. 10A-G illustrates example atomizers for use in the delivery platform of FIG. 1.

FIGS. 11A-11E are CAD drawings illustrating an example spray head base mounting platform of the delivery platform of FIG. 1.

FIGS. 12A-12D are CAD drawings illustrating an upper portion of an exemplary embodiment of a pod locating nest of the delivery platform of FIG. 1.

FIGS. 13-13C are CAD drawings illustrating a lower portion of an exemplary embodiment of a pod locating nest of the delivery platform of FIG. 1.

FIGS. 14A-14F are CAD drawings illustrating an example pod nest cover of the delivery platform of FIG. 1.

FIG. 15 is an image of an example embodiments of a pod assembly for use in the delivery platform shown in FIG. 1.

FIGS. 16A-16C are images of example embodiments of components of the pod assembly shown in FIG. 15.

FIG. 16D is another example embodiment of the pod assembly of FIG. 15.

FIG. 17 is an isometric view of a CAD drawing illustrating an exemplary embodiment of a pod assembly within a pod nest of the delivery platform of FIG. 1.

FIG. 18 is a cross-sectional view of the exemplary embodiment shown in FIG. 17.

FIGS. 19A-19C are CAD drawings illustrating example embodiments of a filter plate coupling of the pod assembly of FIG. 15.

FIG. 20 is a flow diagram illustrating an example embodiment of a process for delivery to cells using the delivery platform of FIG. 1.

FIG. 21A-B illustrate example frames for stacking and processing pods.

FIG. 22 illustrates an example spray-guard according to some example implementations.

FIG. 23 illustrates an image of another example embodiment of a delivery platform according to some embodiments disclosed herein.

FIG. 24 illustrates a view of the platform shown in FIG. 23.

FIG. 25 illustrates a second view of the platform shown in FIG. 23.

FIG. 26 illustrates a close-up view of a portion of the platform shown in FIG. 23.

FIG. 27 illustrates an image of an example embodiment of a single-use assembly of the delivery platform shown in FIG. 23.

FIG. 28 illustrates an image of an example embodiment of a spray head of the single-use assembly shown in FIG. 27.

FIG. 29 illustrates a schematic of the experimental design for simultaneous delivery of RNPs. Cas9 RNP-TRAC sgRNA was prepared at 2:1 ratio at 0.4 μg/μL (equiv to 3.3 μg per 1×10⁶ cells); S Buffer solutions were prepared with 0, 5, 10 and 15% ethanol with RNP and the experiments were carried out on the delivery system with the S buffer solutions at each ethanol concentration.

FIG. 30 illustrates representative flow cytometry plots from cells stained with an antibody targeting CD3 (gated off the live population). Untreated (UT) cells showed >93% positivity for CD3 and this was reduced following delivery of TRAC RNP by the example delivery platform illustrated with respect to FIG. 1. Two distinct populations are observed in the treated samples with the population on the left (gated) referring to those cells that were negative for CD3 staining. This negative population increased from ~59% in samples where no ethanol was present in the delivery Solution to ~67% in samples where ethanol was present. A limit exists to the amount of ethanol present before precipitation of the Cas9 protein occurs (>20% ethanol at 0.4 μg/μL Cas9 RNP).

FIG. 31A is a bar graph showing the mean CD3 negative population (±standard deviation) from 2-3 replicates per condition in activated T cells 72 hr post-delivery of TRAC RNP (2:1 guide to Cas9 molar ratio; 3.3 μg per 1×10⁶ cells) by the example delivery platform illustrated with respect to FIG. 1. Increasing concentrations of ethanol were added with the cargo in the delivery solution. The level of CD3 edit increased modestly with increasing concentrations of ethanol (0% EtOH-58% to 15% EtOH-66%). “UT” refers to untreated control cells.

FIG. 31B is a table showing the mean, standard deviation, standard error of the mean and coefficient of variation of CD 3 negative expression from each group 72 hr post-delivery of TRAC RNP by the example delivery platform illustrated with respect to FIG. 1.

FIG. 32 is a bar graph depicting the percent viability at the increasing ethanol concentrations, and time points consisting of pre-delivery, post-delivery (day 3) and post-delivery (day 5).

FIG. 33A is a line graph showing that aqueous solutions without ethanol show a larger droplet size for the same pressure as compared to a solution containing ethanol. As shown in the graph, for achieving spray particle size similar to cells of approximately 10 μm in diameter (e.g., human T cells), the spray droplet size requires higher atomization pressures to be applied to maintain the droplet size range closer to the cell size, including to avoid excessively large droplets.

FIG. 33B is a line graph showing that aqueous solutions with ethanol show a smaller droplet size (as compared to aqueous solutions without ethanol for the same pressure).

FIG. 34A and FIG. 34B are bar graphs showing that an increase in GFP transfection was achieved using 12% ethanol in solutions and increasing the proportions of sucrose and sodium chloride from the two buffer solutions. The cell viability was also maintained.

FIG. 35A and FIG. 35B are bar graphs showing that an increase in GFP transfection was achieved using 27% ethanol in solutions and increasing the proportions of sucrose and sodium chloride from the two buffer solutions. The cell viability was also maintained. Like reference symbols in the various drawings indicate like elements.

FIG. 36 is a line graph showing a linear regression analysis demonstrating that the osmolal gap was solely due to ethanol, based on the difference between measured serum osmolality after ethanol addition and measured serum osmolality before ethanol addition and serum ethanol concentration in mg/dL. Osmolal Gap (mOsm/kg H2O)=0.234 (Ethanol [mg/dL])−1.427 (95% CI: slope 0.226-0.243, intercept −2.971 to 0.118). FIG. 36 is reproduced from Nguyen, M. et al “Front. Med. Is the Osmolal Concentration of Ethanol Greater Than Its Molar Concentration? Jan. 8, 2020, “Nguyen” incorporated herein by reference in its entirety).

FIG. 37 is a bar graph showing that hypertonic solutions increase transfection.

FIG. 38 is a bar graph showing the effect of the hypertonic solutions on viability.

FIG. 39 is a schematic of the delivery system and method for induced pluripotent stem cells (iPSCs).

FIG. 40 are graphs depicting the increased transfection efficiency of the delivery system with iPSCs.

FIG. 41 is a schematic depicting the delivery system improvements using iPSCs.

FIG. 42 are graphs that the methods delivered functional cargo to primary T cells. The graph depicts dosing studies of RNPs and a dose response in all 3 targets was observed. Typically 3 ug/million cells are delivered. Data showed how the methods delivered to T cells and KO a variety of industry-relevant targets including TRAC, CD7 and B2M. Cell Type: T cells; Cargo: Single RNP (TRAC, CD7, β2M) Number of Donors: 1; Post-process cell viability >80%.

FIG. 43 are data showing that the methods demonstrated minimal perturbation of T cells. Cell quality was a critical element. A marked difference in the response of cells to EP than to the described methods was observed. Minimal perturbation means the cells will behave as physiologically relevant as possible.

FIG. 44 are graphs showing that the methods delivered multiplex and sequential complex edits. In sequential multiplex transfections the methods demonstrated higher edited cell yield, with superior cell function vs electroporation. In addition, the methods supported sequential delivery. This dataset showed that the same population of cells was transfected 24 hours post the first transfection. This sequential delivery showed the superior cell function of T cells against the comparator. A shorter time point of 1-4 hours demonstrated excellent functionality data.

FIG. 45 are images and graphs demonstrated that the methods enhanced in vivo cell functionality. Human primary T cells were engineered with CAR mRNA. These cells were injected into the mice and monitored over a 15-day period. The images depict that at the highest dose of cells processed using the methods, the tumor was eradicated in some of the samples. There is a higher evidence of disease-free mice. In addition, and from the graph on the right, cells processed using the method had far superior engraftment in comparisons to CAR-T cells that were processed using EP (electroporation). This again supports the in vitro studies where it was demonstrated the superior functionality of cells processed using the method.

FIG. 46 depicts a schematic of the workflow for in vivo mouse studies.

FIG. 47 are data demonstrating potent targeted cytotoxicity of the methods. Triple KO CAR-T Cells demonstrated potent targeted cell cytotoxicity when co-cultured with CD19+ Raji cells. Cells processed using the method had less apoptotic cells in comparison to electroporation and showing superior cell health post process 25% of the remaining cells from electroporation went through apoptosis whereas the methods, only 12.5% went through apoptosis. This is measured using a dye that tracks caspase-3 activity to monitor apoptosis in the early and late stages. In addition, the methods demonstrated a higher % of stem cell memory T cells retained post process in comparison to EP which is the type of population cell therapeutic developers are looking for. This was measured 4 days post transfection, which is 2 days post transduction. This was monitored to Day 7, as well as being measured immediately after 4 hours post-transfection, where the phenotype was unchanged and similar to the untreated population, whereas nucleofection decreased.

FIG. 48 are data demonstrating the superior cell health and phenotype of the methods. CAR-T Cells engineered using the system were less apoptotic than electroporation, retained a younger memory phenotype, and were more metabolically active, with a higher maximum respiratory rate (oxygen consumption rate).

FIG. 49 is a graph showing that the oxygen consumption rate (OCR) was the highest in the claimed methods (as a measure of cell fitness and metabolism). System; Cell Type: T cells; Cargo: Single RNP (TRAC, CD7, β2M) Cargo Loading: 3 μg/M cells (each RNP) Number of Donors: 3; T cells activated 3 days post-editing; LVV transduction 2 days post-transfection; CAR-T expression per sample=45%.

FIG. 50 are bar graphs showing that the methods and system did not impact cell health during post-transfection processes (left—transduction and right—post thaw after cryopreservation). System: Cell Type: T cells; Cargo: Single RNP (TRAC, CD7, β2M) Cargo Loading: 3 μg/M cells (each RNP) Number of Donors: 3; T cells activated 3 days post-editing; LVV transduction 2 days post-transfection; CAR-T expression per sample=45%.

FIG. 51 is a graph showing that the methods demonstrated potent in vivo cell functionality. System: Cell Type: T cells; Cargo: Single RNP (TRAC, CD7, β2M) Cargo Loading: 3 μg/M cells (each RNP) Number of Donors: 3; Number of Mice: 8. T cells activated for 3 days. Transfection followed by; LVV transduction (2 days post-transfection); Cryopreserved 24 hrs post transduction; CAR-T expression per sample=80%. Data displayed as mean+/−SEM (N=8).

FIG. 52 are graphs demonstrating that the methods showed dose dependent protection against tumor growth. NSG mice were inoculated with Raji-Luciferase cells, a CD19+ cancer cell line. Note: NSG=completely lack a functional mouse immune response, and therefore have no immune system. 4 days after inoculating with Raji cells, the mice are dosed with triple knock out CAR-T cells generated using either Solupore or electroporation (compared to untransfected control CAR-T cells), the cancer cells are left to grow over a X amount of days within each treatment arm over three cohorts of doses (1e6/mouse, 2e6/mouse, 4e6/mouse). As the dose of CAR-T cells were increased in each cohort of mice, the method demonstrated a dose dependent tumour growth inhibition similar to the untransfected control group, as opposed from Electroporation/standard technology which fails to control tumour growth in all three respective dosing groups, the tumour burden between the method and electroporation in the higher dose is statistically significant (p val=0.0006), whereas the difference between Sol and UT is not significant (pval=0.06). System: Cell Type: T cells; Cargo: Single RNP (TRAC, CD7, β2M) Cargo Loading: 3 μg/M cells (each RNP) Number of Donors: 3; Number of Mice: 8. T cells activated for 3 days. Transfection followed by; LVV transduction (2 days post-transfection); Cryopreserved 24 hrs post transduction; CAR-T expression per sample=80%. Data displayed as mean+/−SEM (N=8).

FIG. 53 are graphs showing the translatability of the methods and system from the research tool to the single use system. System: Cell Type: T cells; Cargo: Multiplex RNP (TRAC, CD7, β2M); Cargo Loading: 3 μg/M cells (each RNP); Number of Donors: 3-5; T cells activated 3 days post-editing.

FIG. 54 are graphs showing that the transfection methods demonstrated enhanced stem cell memory and cytotoxic function in engineered T cells expanded for 7 days to >1.3×10e9. Top T cell proliferation in 6M G-REX plates comparing untransfected control to Solupore transfected cells (Cas9 RNP targeting TRAC). Bottom—stem cell memory subpopulation (T_{SCM}) measured by flow cytometry.

FIG. 55 are graphs demonstrating the superior cell health and phenotype of the methods and systems. CAR-T Cells engineered using the system retained a younger memory phenotype, and expressed functionally relevant cytokines after additional stimulation. System: Cell Type: T cells; Cargo: Multiplex RNP (TRAC, CD7, β2M); Number of Donors: 3; Cargo loading, 3 μg/M cells (each RNP).

FIG. 56 are graphs showing that the methods demonstrated superior ability to bind target cells after cryopreservation. Cell avidity: the overall strength of interactions between a diversity of receptor-ligand pairs at the cell surface. Small shifts in avidity (5-10%)=drastic performance changes in vivo. System: Cell Type: T cells; Cargo: Single RNP (TRAC, CD7, β2M) Cargo Loading: 3 μg/M cells (each RNP) Number of Donors: 3; T cells activated 3 days post-editing; LVV transduction 2 days post-transfection; CAR-T expression per sample=45%; ** indicates P value of 0.0032 determined by ordinary one-way ANOVA.

FIG. 57 is a graph showing the system and methods efficiently transfected a variety of cell types including NK cells. Transfected cells maintained high viability. The system and methods are agnostic to cell types. Transfection of both iPSC and NK is shown with a variety of cargo leading to excellent efficiency and viability post process. iPSC model cargo GFP mRNA and post-process had a healthy cell culture post process with a good expansion. NK-TIGIT KO.

FIG. 58 are graphs demonstrating that the methods efficiently transfected a variety of cell types.

FIG. 59 are graphs showing that the system and methods effectively transfected iPSCs. The method demonstrated high edit efficiency and viability for GFP mRNA and RNPs. Using the method, the cells are visibly healthier than cells gene edited using electroporation. GFP mRNA-3 μg per 1E6 cells (5E6 cells total)—24 hr assessment (flow cytometry); B2m RNP—10 μg per 1E6 (5E6 cells total)—96 hr assessment (flow cytometry); Viability—Chemometic nucleocounter Via 1 cassette; Sequential spray timing—1 hr between sprays; All Exp with 37 °C Stop Solution; Building towards unique platform for concurrent Parental/Edited iPSC MCB;—Blood derived parental & edited MCBs in <3 months;—Strong safety profile: Non-integrating & <off targets;—Adaptable to novel targets & HSC gene engineering.

FIG. 60 are graphs showing that the system and methods efficiently transfected CD34+ cells with multiple mRNA deliveries. GFP mRNA (3.3 μg per 1E6) delivered by RT to 5e6 CD34+ cell on day 0, day 2 and day 4. Cells were pooled between each transfection and 5e6 transfected each time. GFP expression assessed by flow 2 days post each transfection. Viability assessed after each transfection. The data demonstrated the potential for CD34+ reprogramming to iPSC using multiple mRNA deliveries.

FIG. 61 are bar graphs showing that the system and methods delivered large plasmid payloads while maintaining cell viability. It was demonstrated that the system and methods are capable of delivering GFP plasmid to T cells in two concentrations with excellent viability post-process. Even with doubling the concentration of plasmid y good viability growth is maintained.

FIG. 62 are graphs showing that the system and methods demonstrated good knock-in efficiency with excellent viability across 2 donors. Cells were activated by Transact for 2 days prior to transfection with IL-7 and IL-15. Cells were pre-treated for 30 min prior to transfection with NATE (innate immune response inhibitor); 6E6 transfected on Day 0; Cargo: TRAC RNP—6 μg per 1E6; 4:1 sgRNA to Cas9; ssDNA CTS eGFP from Genscript—4 μg per 1E6; RNP+ssDNA complexed prior to delivery (theory—NLS on Cas9 transports ssDNA into nucleus). Cells seeded into 24-well G-Rex plate and spiked with M3814 (HDR Enhancer; 1 μM) and dNTPs (50 μM) for 24 hr Cell counts carried out on Day 1, 4 and 7. GFP expression assessed on Day 4 and 7. System: T cells, TRAC RNP—6 ug per 1E6; 4:1 sgRNA to Cas9. ssDNA CTS eGFP—4 ug per 1E6.

FIG. 63 is a table showing various donors and cell types for reprogramming blood cells to iPSCs.

FIGS. 64A and 64B are images showing CD34+ cells transfected with mRNA for reprogramming. Suspension culture at 6 hours is shown in FIG. 64A and attached culture at 4 hours is shown in 64B.

FIG. 65 is a table depicting iPSC clone characterization.

FIG. 66 is an image depicting CD34+ and PBMC cells stained for pluripotency markers and differentiation markers.

FIG. 67 is an image of CD34+ cells stained for pluripotency markers.

FIG. 68 are images showing reprogramming of CD34+ cells following delivery of srRNA and seeded at various cell concentrations. Reprogramming was confirmed in 3 out of 3 donors tested.

FIG. 69 is a bar graph depicting mRNA transfection of iPSCs.

FIG. 70 is a bar graph depicting sequential editing of iPSCs.

FIG. 71 is an image showing the knock-in of a reporter molecule in iPSCs.

FIG. 72 is a schematic showing the gene editing of iPSCs overview.

FIG. 73 is a schematic showing the Solupore non-viral delivery technology. The technology is a non-viral system and delivers cargo physiochemical means, yielding cells with superior cell health and improved proliferative capacity. The delivery method has previously demonstrated efficient sequential gene modification in T and NK cells, but not iPSCs.

FIGS. 74A-74B are schematics showing the multi target capabilities of the delivery method and system in iPSCs. FIG. 74A is a bar graph showing day 3 gene editing efficiency using TIDE analysis of pooled iPSC transfected with CRISPR/Cas9 RNP using the revised SOLUPORE protocol. FIG. 74B is a bar graph showing gene editing efficiency using TIDE analysis of pooled iPSC transfected with various CRISPR/Cas9 RNP conditions.

FIGS. 75A and 75B are images showing the high cell viability and transfection efficiency of the delivery method and system in iPSCs. FIG. 75A is a bar graph showing Day 0 viability of iPSCs transfected with GFP-mRNA and CRISPR/Cas9 RNP using the delivery method and system and a nucleofector (NF). FIG. 75B is data showing the assessment of GFP+ transfection in iPSCs 24 hours post-transfection using the delivery method and a NF using (a) fluorescent imaging and (b) flow cytometry. iPSCs were dissociated and subjected to transfection with GFP-mRNA or CRISPR/Cas9 RNP using SOLUPORE and a commercial NF. Cell viability was assessed on Day 0 post-transfection, and efficiency (% GFP+) was assessed 24 h later, using Flow cytometry. Both NF and SOLUPORE had comparable post-transfection viability (80+%) regardless of the cargo delivered (GFP mRNA or CRISPR/Cas9 RNP). Comparable GFP+ (90+%) cells were observed between SOLUPORE and NF on Day 1 post-transfection.

FIG. 76 is a bar graph showing the delivery method for iPSCs had higher single cell seeded colony survival at day 1 and reduced manufacturing time and cost. CRISPR/Cas9 transfected iPSCs with delivery method and NF were subjected to single cell clone isolation, simulating our clean room workflow on Day 1 and Day 4 post transfection (schematic). The delivery method had a 2.5× fold cell survival when subjected to single cell seeding at Day 1 post transfection compared to NF. Single cell clone seeding on Day 1 compared to Day 4 reduces clean room time usage and cost.

FIGS. 77A and 77B are bar graphs showing that the delivery method and system had higher cell recovery post CRISPR/Cas9 transfection but lower gene editing efficiency using T/NK cell adapted for transfection protocol. FIG. 77A is a bar graph showing day 3 cell growth of pooled iPSC transfected with CRISPR/Cas9 RNP using the delivery method and a NF. FIG. 77B is a bar graph showing Day 3 gene editing efficiency using TIDE analysis of pooled iPSC transfected with CRISPR/Cas9 RNP using the delivery method and a NF. CRISPR/Cas9 RNP, targeting B2M gene, transfected iPSCs were assessed for cell count and gene editing efficiency, using TIDE analysis, 1 on Day 3 post-transfection. The delivery method had a 2× fold higher cell recovery at Day 3 post-transfection compared to NF. The delivery method protocol for T/NK cell transfection had lower gene editing efficiency than the NF at Day 3 post transfection.

FIG. 78 are flow cytometry images for tdTomato (inserted DNA template) in pooled transfected iPSCs, using the delivery method and NF.

### DETAILED DESCRIPTION

Despite some advances, delivery of certain particles and/or molecules into cells remains a challenge. Factors such as size or charge of a molecule to be delivered into a cell can limit and/or prevent delivery of the molecule into the cell. In particular, delivery across the cell membrane can be complicated due to the molecule and/or the membrane of the cell. A cell membrane or plasma is a semi-permeable biological membrane, which acts as a selective barrier. The membrane regulates an internal chemical composition of the cell. As the selective barrier for the cell, the membrane can allow only certain molecules to passively translocate across the membrane through, for example, passive diffusion into the cell. Small, hydrophobic molecules (such as O₂, CO₂, and N₂) and small, uncharged polar molecules (such as H₂O and glycerol) can passively diffuse across cell membranes. Larger, uncharged polar molecules (such as amino acids, glucose, and nucleotides) and ions (such as H⁺, Na⁺, K⁺ and Cl⁻) cannot passively diffuse across cell membranes.

Reversible permeabilization can be used for intracellular delivery of compounds in clinical settings, as well as in research and development environments. For example, in clinical or therapeutic treatment methods, cells can be extracted from a patient, isolated (e.g., concentrated or enriched), and subsequently be treated with the cell engineering methods. The engineered cells can be expanded and returned to the patient. For delivery across cell membranes, methods using viral vectors can be used. However, the methods based on viral vectors generally require high costs and complex processes, provide limited accessibility, and offer variable and inconsistent results. Methods based on electroporation can also be used. However, the electroporation-based methods generally result in higher cell damage and offer poor cell recovery and cell functionality.

In implementations, the present disclosure relates to methods of reprogramming blood cells (e.g., mature adult cells such as PBMCs or immature cells such as CD34+ cells) to iPSCs. Transfection of CD34+ cells and PBMCs using reprogramming factors delivered as mRNA or srRNA demonstrated non-viral reprogramming of the blood cells. The described method and delivery system allows reprogramming of blood cells, including CD34+, using srRNA, and thus obviating the need for Sendai virus (70-100 day clearance) and reducing manufacturing time and cost.

The present disclosure relates to using an increased environmental temperature when operating the delivery system and process. Some implementations of the subject matter relate to increasing the environmental temperature of the platform, wherein the process is performed at the increased temperatures. In some examples, the system and process is performed above ambient temperature (above about 20-25 °C). In some examples, the delivery system, may be placed in a temperature-controlled incubator to control and monitor the temperature of the entire system. For example, the delivery system (and the components thereof) is placed in an incubator and set at a temperature of about 37 °C. In other examples, the delivery system is placed in an incubator and the incubator is set at a temperature of about 30 °C, about 31 °C, about 32 °C, about 33 °C, about 34 °C, about 35 °C, about 36 °C, about 37 °C, about 38 °C, about 39 °C, about 40 °C, about 41 °C, about 42 °C, about 43 °C, about 44 °C, about 45 °C, about 46 °C, about 47 °C, about 48 °C, about 49 °C, or about 50 °C. By utilizing the delivery platform for delivery of payload to cells at higher temperatures, cell viability can be increase relative to utilizing the delivery platform for delivery of payload to cells at ambient temperatures. And increasing cell viability after delivery provides for increased ability to review and assess the efficacy of the payload upon delivery across the plasma membrane of the cell.

In some examples, when the delivery system is placed in an incubator and set at a temperature of about 37 °C, the temperature of the components (e.g., stop solution, delivery solution, or aqueous solution) of the system and method are also increased. In some examples, the temperature of the stop solution is increased from ambient temperature (e.g., of about 20-25 °C) to about 37 °C, or about 30 °C, about 31 °C, about 32 °C, about 33 °C, about 34 °C, about 35 °C, about 36 °C, about 37 °C, about 38 °C, about 39 °C, about 40 °C, about 41 °C, about 42 °C, about 43 °C, about 44 °C, about 45 °C, about 46 °C, about 47 °C, about 48 °C, about 49 °C, or about 50 °C.

In some examples, the temperature of the delivery solution is increased from ambient temperature (e.g., of about 20-25 °C) to about 37 °C, or about 30 °C, about 31 °C, about 32 °C, about 33 °C, about 34 °C, about 35 °C, about 36 °C, about 37 °C, about 38 °C, about 39 °C, about 40 °C, about 41 °C, about 42 °C, about 43 °C, about 44 °C, about 45 °C, about 46 °C, about 47 °C, about 48 °C, about 49 °C, or about 50 °C. In some examples, the temperature of the aqueous solution is increased from ambient temperature (e.g., of about 20-25 °C) to about 37 °C, or about 30 °C, about 31 °C, about 32 °C, about 33 °C, about 34 °C, about 35 °C, about 36 °C, about 37 °C, about 38 °C, about 39 °C, about 40 °C, about 41 °C, about 42 °C, about 43 °C, about 44 °C, about 45 °C, about 46 °C, about 47 °C, about 48 °C, about 49 °C, or about 50 °C.

Some implementations of the current subject matter can provide solution-based delivery to address the cost and complexity challenges for the cell engineering technologies. To provide a reliable and consistent method for cell therapies, the current subject matter can provide a cell engineering method and platform to deliver compounds or mixtures of compounds (e.g., payload) into cells across cell membranes by contacting the cells with a delivery solution containing the payload. The cells may be suspension cells or adherent. In some implementations, the delivery of payload into cells across cell membranes can be performed by including in the solution an agent for reversibly permeabilizing cell membranes, which can also be referred to as a cell poration process. Further, poration of cells can refer to a process of permeabilizing cell membranes and delivering payloads across cell membranes into cells.

Some implementations of the current subject matter can provide a platform for cell engineering that can provide clinical grade transfection in that treated cells have high viability and expression. In addition, the delivery platform can provide smaller scale cell processing and can be used for experimental designs involving smaller quantities of cells, such as 0.5M-15M cells. The platform can include features that make it easy to use, for example, by having single-use pods for performing the cell engineering process that is described in more detail herein. In some embodiments, the pod can be reusable. In some embodiments, the pods can be chamber. The system can include control features enabling easy to implement and repeatable cell processing. Some implementations can be particularly useful, for example, in research and development efforts. The platform can also be used for vector-free delivery of payload/cargo compounds and compositions into non-adherent cells. In some embodiments, the delivery platform can provide for quantities of cells including from about 150,000 to about 15M cells. In some embodiments, the delivery platform provides for quantities of cells from about 100,000 to about 500,000 cells, or about 150,000 to 400,000 cells, or from about 150,000 to about 300,000 cells, or from about 150,000 to about 200,000 cells. In some embodiments, the delivery platform provides for quantities of cells of about 500,000 cells.

Using the platform of the present disclosure, other cell engineering processes may also be performed before and/or after the delivery process, which can significantly enhance productivity and allow the overall process to be streamlined. Moreover, not only the non-viral transfection method but also viral methods may be performed within the single platform.

The delivery platform described herein can achieve delivery of a payload across a plasma membrane of a non-adherent cell by performing the steps of providing a population of non-adherent cells and contacting the population of cells with a volume of an isotonic aqueous solution, the aqueous solution including the payload. In some implementations, the aqueous solution does not include an alcohol (e.g., the solution is in the absence of alcohol (e.g., 0% ethanol)). In some implementations, the solution can also include an alcohol at greater than 0.2 percent (v/v) concentration. For example, the alcohol comprises ethanol (e.g., greater than 5% ethanol, greater than 10% ethanol, and the like). In some examples, the aqueous solution comprises between 20-30% ethanol, e.g., 27% ethanol. Other compositions are possible.

The current subject matter can also provide a platform that can automate the cell poration process and allow delivery to cells to be performed at a various scales. When cells are manually loaded to the platform and/or manually unloaded from the platform, the throughput of the system is limited, difficulties exist in applying to clinical process/treatment. There may be concerns for contamination and inconsistent process depending on operators and/or various environmental parameters. By the process automation, the delivery process can be performed more consistently, a concern for contamination can be significantly reduced, and therefore, the system can be scaled more easily. Exemplary embodiments of the delivery platform to perform the delivery process with manual and automated processes will be described.

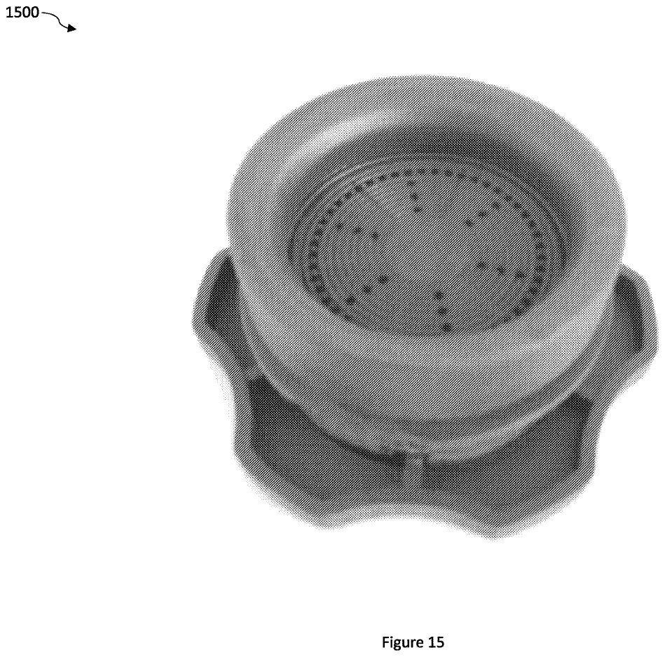

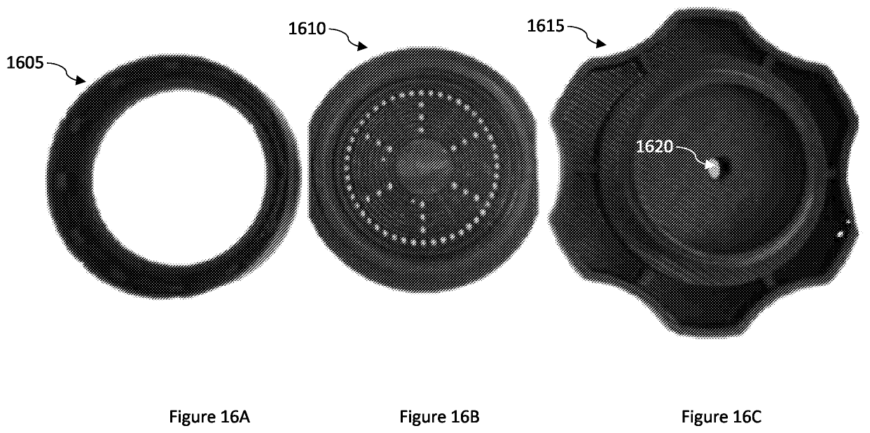

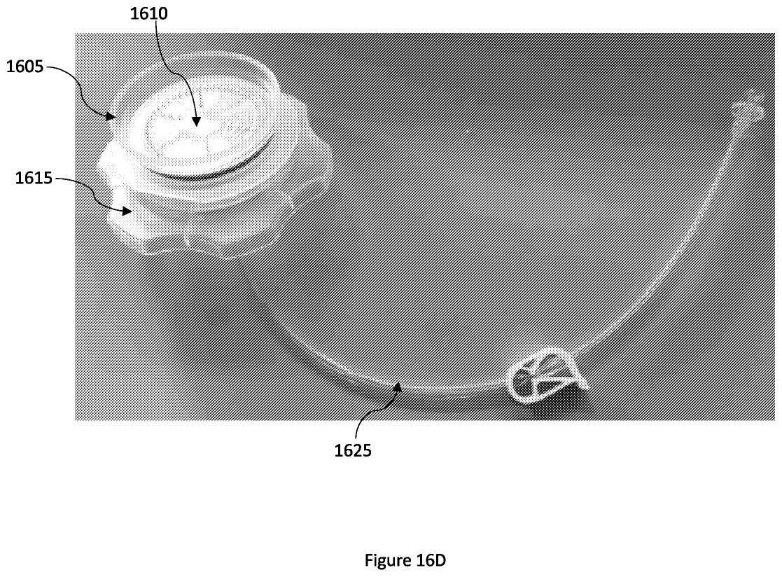

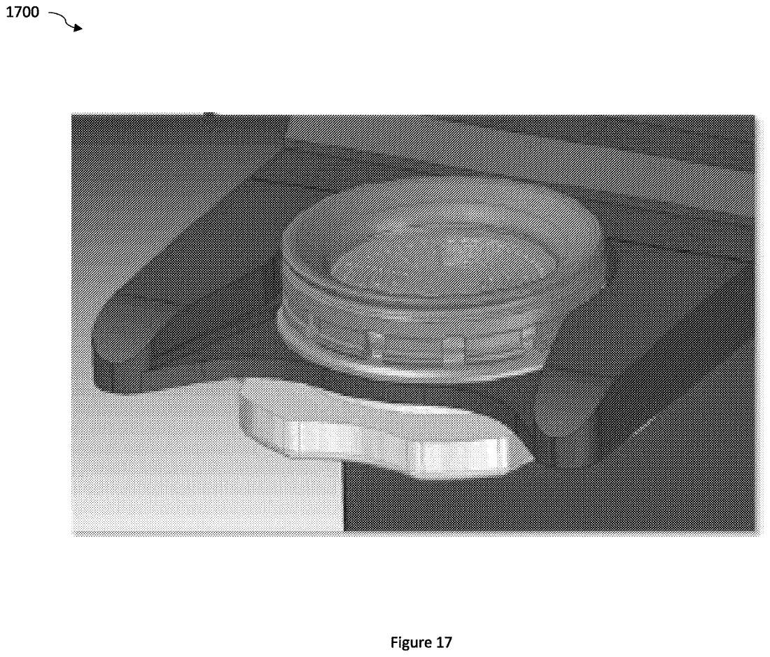

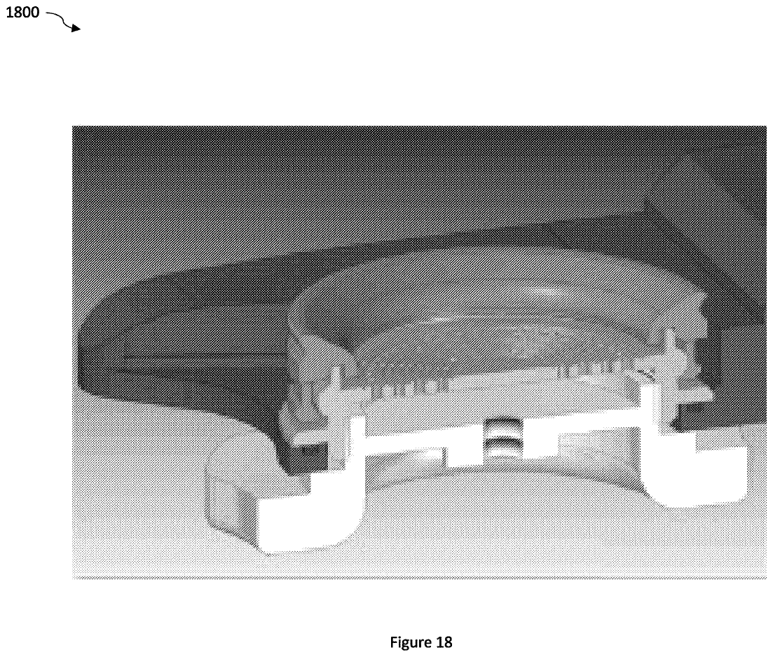

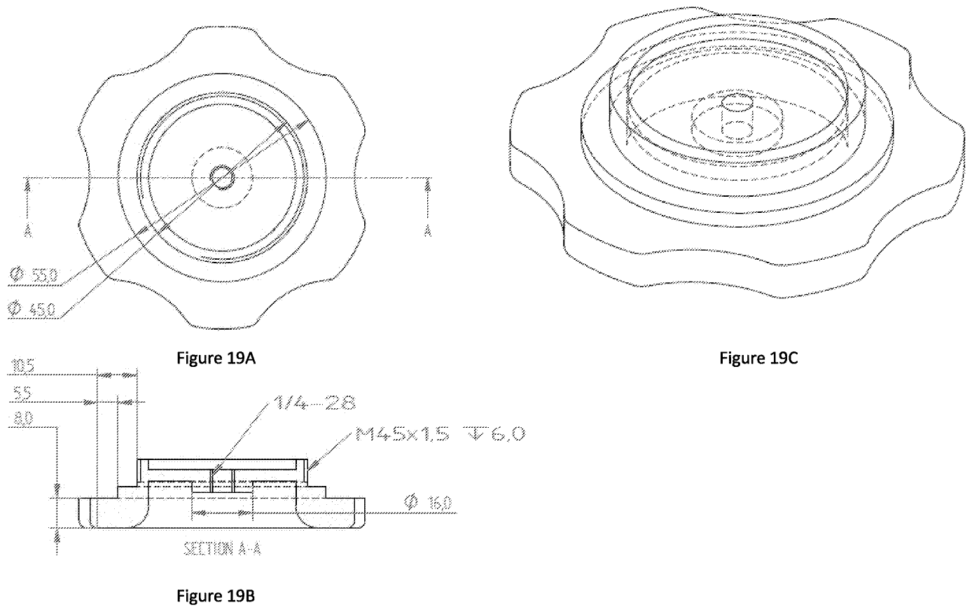

An example pod according to some implementations is shown in FIGS. 15-19 and is described more fully below. The example pod includes an upper portion 1605, a filter plate 1610, and a lower portion 1615. In some implementations, pods may be designed for specific cell populations and sizes. For example, pods can include different lower portions based on the culture. As used herein, the pod can be referred to as a chamber, a chamber assembly, a single-use assembly, or a disposable assembly, for example.

In some implementations, the pod may be manufactured as a single molding rather than having multiple parts that clip together. The pod may have its filter membrane bonded into this single substrate. The pod may have a filter with a smaller diameter such that a smaller population of cells may be treated. The pod may have markings molded into it to indicate fill level or have molded features to ensure orientation within the platform is consistent. The pod may have multiple features to enable it to be retained within a pod holder or stack outside of the apparatus. The pod may have a lid feature to facilitate incubation of cells within it. The pod may have a one-way check valve implemented to enable culture medium to be maintained within the cavity beneath the filter, or to support culture medium above the filter medium to keep cells in suspension post use of the pod.

As another example, some pods can include a hydroscopic foam located in the lower portion for pulling fluid from above the filter plate. Such an approach can be used to pull a delivery and/or payload solution off a cellular monolayer formed above the filter plate, thereby controlling a length of contact between the cell population and the delivery and/or payload solution. An example foam is 3M™ Tegaderm™ Foam Dressing (non-adhesive).

As another example, the lower portion does not include holes and can include a flat tissue cultured treated surface. Such an implementation can be suitable for adherent cell populations to enhance adherence. Such an implementation with a flat surface can be utilized for delivery to tissue explants or engineered tissues.

In some implementations, the pod can be suitable for culturing cells. Rather than immediately removing the cells from the pod, the cells can be cultured for a period of time, such as hours or days. In such implementations the pod can be formed of culture compatible materials and a pod lid can be provided.

In some implementations, the pod can include memory storing process parameters. For example, a pod memory can be programmed with the process parameters such that, when the pod is inserted into the cell engineering platform, a controller on the cell engineering platform reads, from the pod memory, the process parameters. The cell engineering can proceed using the process critical parameters, for example, via an automated fashion (e.g., an amount of solution delivered to the cells can be determined by the controller), or via displaying instructions to the user via a display. By having the process parameters stored on the pod prior to conducting the delivery process, repeatability can be improved because the user is not required to enter the process parameters into the platform.

In another example, the process parameters are first loaded into the controller of the cell engineering platform, and the delivery process is performed using those parameters. After completion of the process, the cell engineering platform can write to the pod memory the process parameters for future reference. These process parameters can include any parameter utilized or described herein as related to delivery of a payload into a cell. For example, the delivery protocol such as solution compositions, exposure lengths, incubation times, wash cycles, temperatures, spray characteristics, pressures, volumes (e.g., of delivery solution to be applied, media to introduce, and the like), cell characteristics, and the like.

In some implementations, the cell engineering platform can write information such as an experiment identifier, date, time, and the like, to the pod memory for future use and/or reference. In some implementations, pods can communicate with one another. For example, a container or housing adapted to hold multiple pods can include connections between the pods so that the container reads data from the memory of a first pod, and copies some or all of the data to the other pods contained in the container. Such an approach can also improve repeatability because, once the first pod is programmed with process critical parameters, that data is replicated to the other pods without modification to some or all of the data.

In some implementations, the pod can include a memory, a processor, and/or a communications module, such as a near-field or radio frequency identification (RFID) communication module capable of communicating with the cell engineering platform and/or other pods. In some implementations, the pod can include electrical contacts for communicating with the cell engineering platform when the pod is inserted into the cell engineering platform. Other implementations are possible.

### Blood Cell Non-Viral Reprogramming

Aspects of the present subject matter obviate the need for viruses to reprogram blood cells to iPSCs. To provide iPS cells from alternative sources in addition to dermal fibroblasts commonly used in the current art, methods for reprogramming a cell population comprising peripheral blood cells or CD34+ may be provided. As described herein, the blood cells are reprogrammed without the need for viruses and thus avoid a long lapse time for viral clearance.

The population of blood cells can be reprogrammed using by transfecting the blood cells for delivery of a payload (e.g., mRNA or srRNA) without the use of a viral vector. As described herein, the method includes transfecting the population of cells by contacting the population with a volume of an aqueous solution including a payload (e.g., a nucleic acid molecule) and an alcohol at a greater than 2 percent (v/v concentration). In another aspect, the aqueous solution includes the payload, an alcohol at greater than 5 percent concentration, greater than 46 mM salt, less than 121 mM sugar, and less than 19 mM buffering agent. For example, the alcohol, e.g., ethanol, concentration does not exceed 50%.

In examples, the population of blood cells is at a density of about 5e4 to about 5e7. In other examples, the population of blood cells is at a density of about 5e4, or about 5e5, or about 5e6, or about 5e7. In aspects, the population of blood cells is at a density of about 5e6 to about 5e7, or from about 5e5 to about 5e6. For example, the cell population may comprise at least, about, or at most, 1×10³, 2×10³, 3×10³, 4×10³, 5×10³, 6×10³, 7×10³, 8×10³, 9×10³, 1×10⁴, 3×10⁴, 4×10³, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶ hematopoietic progenitor cells or any range derivable therein.

In examples, the payload includes a nucleic acid molecule, where the nucleic acid molecule includes an mRNA molecule or an srRNA. srRNA is a type of mRNA molecule engineered to replicate itself within host cells, enhancing protein expression and boosting the immune response, making it a promising tool for vaccines and other therapeutic applications. srRNA is designed to achieve greater protein expression with a reduced dose compared to conventional mRNA. srRNA can sustain protein expression for longer periods. srRNA are based on positive single stranded RNA viruses most commonly alphaviruses such as Venezuelan equine encephalitis virus. Conventional messenger RNA (mRNA) vaccines only produce a finite amount of protein due to the short mRNA half-life. srRNA extends the kinetics of expression by a second open reading frame (ORF) encoding the protein machinery necessary for its own replication. This self-replication dramatically increases both the amount of RNA and the time of expression. Consequently, the amount of protein produced from the initial dose is reduced as compared to conventional mRNA.

“Reprogramming” is a process that confers on a cell a measurably increased capacity to form progeny of at least one new cell type, either in culture or in vivo, than it would have under the same conditions without reprogramming. More specifically, reprogramming is a process that confers on a somatic cell a pluripotent potential. This means that after sufficient proliferation, a measurable proportion of progeny having phenotypic characteristics of the new cell type if essentially no such progeny could form before reprogramming; otherwise, the proportion having characteristics of the new cell type is measurably more than before reprogramming. Under certain conditions, the proportion of progeny with characteristics of the new cell type may be at least about 1%, 5%, 25% or more in the order of increasing preference. In some implementations, a reprogramming kit may be used. Exemplary reprogramming factors include Oct-4, Sox-2, Klf-4, c-Myc, Lin-28, or SV40 Large T Antigen (“SV40LT”).

“Peripheral blood cells” refer to the cellular components of blood, including red blood cells, white blood cells, and platelets, which are found within the circulating pool of blood.

“Hematopoietic progenitor cells” or “hematopoietic precursor cells” refers to cells which are committed to a hematopoietic lineage but are capable of further hematopoietic differentiation and include hematopoietic stem cells, multipotential hematopoietic stem cells (hematoblasts), myeloid progenitors, megakaryocyte progenitors, erythrocyte progenitors, and lymphoid progenitors. Hematopoietic stem cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T-cells, B-cells, NK-cells). The hematopoietic progenitor cells may or may not express CD34. The hematopoietic progenitor cells may co-express CD133 and be negative for CD38 expression. In certain embodiments, certain human hematopoietic progenitor cells may not express CD34, but these cells may nonetheless be converted into iPS cells via the methods disclosed herein. Hematopoietic precursor cells include CD34+/CD45+ hematopoietic precursor cells and CD34+/CD45+/CD43+ hematopoietic precursor cells. The CD34+/CD43+/CD45+ hematopoietic precursor cells may be highly enriched for myeloid progenitors. Various lineages of hematopoietic progenitor cells, such as CD34+/CD43+/CD45+ hematopoietic precursor cells, may be converted to iPS cells via the methods disclosed herein. Hematopoietic progenitor cells also include various subsets of primitive hematopoietic cells including: CD34−/CD133+/CD38− (primitive hematopoietic precursor cells), CD43(+)CD235a(+)CD41a(+/−) (erythro-megakaryopoietic), lin(−)CD34(+)CD43(+)CD45(−) (multipotent), and lin(−) CD34(+)CD43(+)CD45(+) (myeloid-skewed) cells, CD133+/ALDH+(aldehydehehydrogenase) (e.g., Hess et al. 2004; Christ et al., 2007). It is anticipated that any of these primitive hematopoietic cell types or hematopoietic precursor cells may be converted into iPS cells as described herein.

As used herein, the term “stem cell” refers to a cell capable of self replication and pluripotency. Typically, stem cells can regenerate an injured tissue. Stem cells herein may be, but are not limited to, embryonic stem (ES) cells or tissue stem cells (also called tissue-specific stem cell, or somatic stem cell). Any artificially produced cell which can have the above-described abilities (e.g., fusion cells, reprogrammed cells, or the like used herein) may be a stem cell.

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by introducing certain factors, referred to as reprogramming factors.

“Pluripotency” refers to a stem cell that has the potential to differentiate into all cells constituting one or more tissues or organs, or particularly, any of the three germ layers: endoderm (interior stomach lining, gastrointestinal tract, the lungs), mesoderm (muscle, bone, blood, urogenital), or ectoderm (epidermal tissues and nervous system). “Pluripotent stem cells” used herein refer to cells that can differentiate into cells derived from any of the three germ layers, for example, direct descendants of totipotent cells or induced pluripotent cells.

### Example 1: Delivery Platform

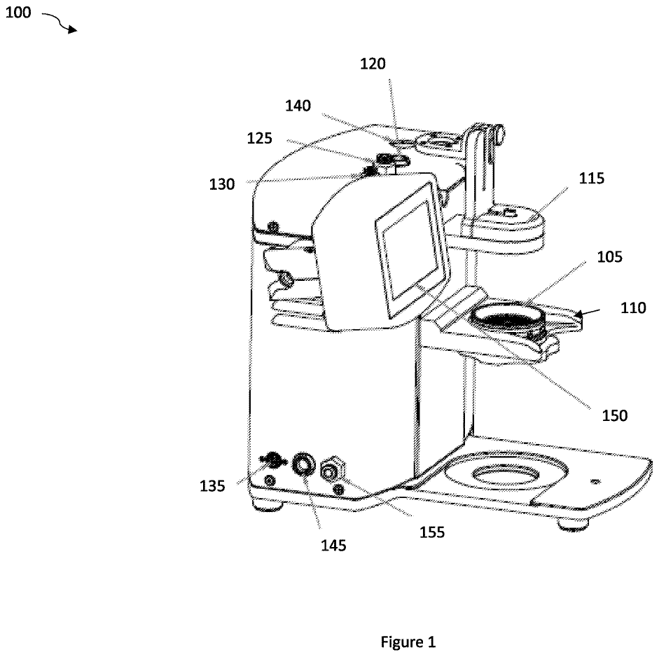

FIG. 1 is an isometric view of a computer aided design (CAD) drawing illustrating an example embodiment of a delivery platform 100 according to some embodiments disclosed herein. The delivery platform 100 includes a pod 105 configured to be received and positioned within a pod nest 110. An example pod 105 is illustrated in FIGS. 15-19. The pod 105 can include an upper portion 1605, a filter plate 1610, and a lower portion 1615. The pod 105 can provide a processing surface, via the filter plate 1610, on which cells can be provided for treatment and processing. For example, the filter plate 1610 can be configured to receive a filter for use in forming a monolayer of cells to be processed using the delivery platform 100.

The pod 105 can be received and positioned within the pod nest 110. In some embodiments, the atomizer nest 115 can be a fixed distance above the pod 105. The atomizer nest 115 can be a fixed distance from the pod nest 110 to reduce the number of variables or degrees of freedom available to the user thereby providing a system that is easier to use. For example, the atomizer nest 115 can be fixed about 75 mm above the pod 105. The pod nest 110 can include a circular opening to receive the pod 105. A lower portion 1615 of the pod 105 can be mated to the filter plate 1610 by coupling the lower portion 1615 with a portion of the filter plate 1610 extending through the circular opening of the pod nest 110. The pod nest 110 can provide support to the pod 105 and can maintain the position of the pod 105 during cell processing using the delivery platform 100. For example, the pod nest 110 can maintain the position of the pod 105 to ensure the treatment surface of the pod 105, e.g., the filter plate 1610, is sufficiently located to receive adequate amounts of delivery solution.

As further shown in FIG. 1, the delivery platform 100 includes an atomizer nest 115. The atomizer nest 115 can include an atomizer coupled to a delivery solution source configured within the delivery platform 100. The atomizer can atomize the delivery solution to provide the delivery solution to the pod 105 (e.g., in the form of a spray) to process or treat cells configured on the filter plate of the pod 105. The atomizer nest 115 can be coupled to the delivery solution source via a valve connector 120, such as a clippard value connector. The atomizer configured within the atomizer nest 115 can be configured to provide the delivery solution to the pod 105 at a predetermined pressure. The delivery platform 100 also includes a sample pressure connector 125 and an air pressure connector 130. The valve connector 120 serves to control delivery solution application to atomizer. The sample pressure connector 125 pressurizes the gas above the fluid in the Eppendorff reservoir to drive the sample into the atomizer. The gas pressure connector 130 supplies pressurized gas to the atomizer.

The delivery platform 100 also includes a power input 135. In some embodiments, the power input 135 can include a 2 channel direct current (DC) 24V power input 135. The power input 135 can be electrically coupled to the On/Off switch 140. The delivery platform 100 also includes a human machine interface (HMI) cable coupling 145. The HMI cable coupling 145 can be electrically coupled to the HMI 150. The HMI 150 can include a display, at least one data processor, and input devices configured to control operation of the delivery platform 100 and to perform the methods of cell treatment via delivery described herein. In some embodiments, the HMI 150 can include a touch screen interface. In some embodiments, the HMI 150 can include process guides, laboratory timers, and the like. The HMI cable coupling 145 can be configured to couple the HMI 150 to a computing device that is located separately from the delivery platform 100. In this way, data can be imported to or exported from the delivery platform 100.

The delivery platform 100 further includes an air supply coupling 155. The air supply coupling 155 can couple the delivery platform 100 to an air supply. The air supply can be used to provide air, via the air supply coupling 155, for use in configuring an amount of air to be provided with the delivery solution to the pod 105.

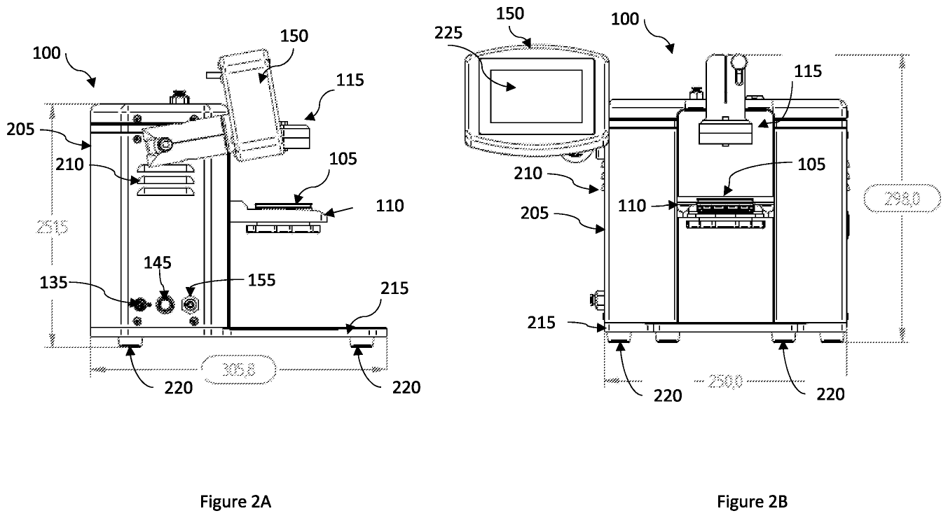

FIG. 2A is a side view of the delivery platform 100 shown in FIG. 1. As shown in FIG. 2A, the delivery platform 100 can include an enclosure 205. The enclosure 205 can include a number of cutouts corresponding to the power input 135, the HMI cable coupling 145, and the air supply coupling 155. Additional cutouts can be provided within the enclosure 205 without limitation. For example, the enclosure 210 can include a plurality of vents 210. The enclosure 205 can be affixed to a base plate 215. The base plate 215 can include a plurality of feet 220. In some embodiments, the feet 220 can be plastic and can include friction-reducing materials to secure the delivery platform 100 on a surface.

FIG. 2B is a front view of the delivery platform 100 shown in FIG. 1. As shown in FIG. 2B, the delivery platform 100 can include an HMI 150 and the HMI 150 can include a display 225. The display 225 can provide visualizations of data and user-interface controls corresponding to one or more aspects of operation of the delivery platform 100. For example, in some embodiments, the display 225 can provide touch screen controls configured to perform one or more operations of methods of delivery to cells. In some embodiments, the HMI 150 can include a timer and the timer, as well as timer controls, can be displayed via the display 225.

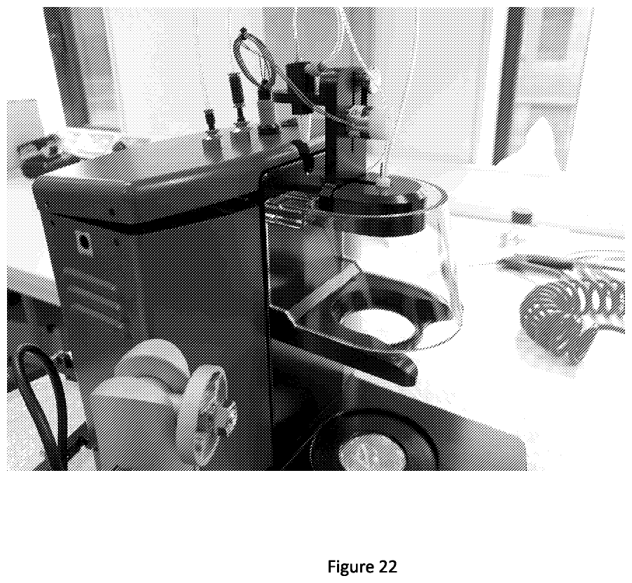

In some implementations, the delivery platform 100 can include a spray-guard device to contain atomization (e.g., overspray). In one example, the spray-guard is transparent, demi-cylindrical device that has the same internal diameter as the outer contour of the pod nest. In some implementations, the spray-guard is not a sealed device but affords some degree of containment. The spray-guard clips on to the front of the device. FIG. 22 illustrates an example spray-guard.

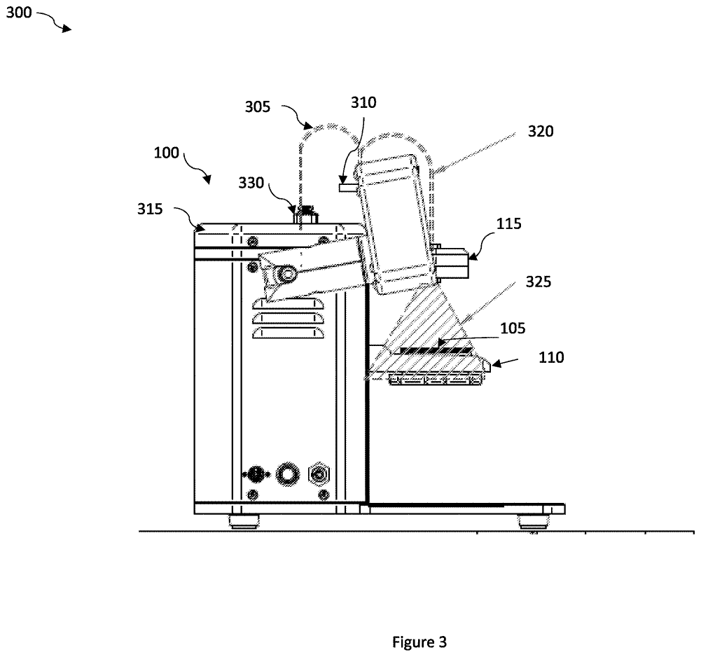

FIG. 3 is a diagram 300 illustrating a side view of another example embodiment of the delivery platform 100 shown in FIG. 1, according to some embodiments disclosed herein. As shown in FIG. 3, the valve can be coupled to the atomizer nest 115 via one or more portions of tubing. A pneumatic fitting 330 can include, for example, a Festo 6 mm to 6 mm bulkhead fitting (Catalogue No. 193951). For example, a first portion of tubing 305 can couple the valve to an Eppendorf base support 310. The Eppendorf base support 310 can be coupled to a top cover 315 of the delivery platform 100.

The Eppendorf base support 310 can include a bracket that holds the payload reservoir in space. An example reservoir includes a 1.5 mL Eppendorf brand centrifuge vial. The reservoir may or may not be permanently fixed in place as the mechanism for securing it to the Eppendorf base support 310.

A second portion of tubing 320 can coupled the Eppendorf base support 310 to the atomizer nest 115. A delivery solution can be conveyed from a source within the delivery platform 100, through the valve and to the Eppendorf base support 310 via the tubing 310. The delivery solution can be further provided to the atomizer nest 115 via tubing 320. Once received within the atomizer nest 115, the delivery solution can be provided to the pod 105 positioned within the pod nest 110. The atomizer configured within the pod nest 115 can be configured to deliver the delivery solution to the pod 105 with a spray pattern 325. The spray pattern 325 can be configurable based on a pressure setting at which the delivery solution is provided. In some embodiments, the spray pattern 325 can be associated with a configuration of an atomizer within the atomizer nest 115. Dimensions of the spray pattern 325, such as a spray angle, a coverage area, and/or a center point can be configurable aspects of the atomizer nest 115.

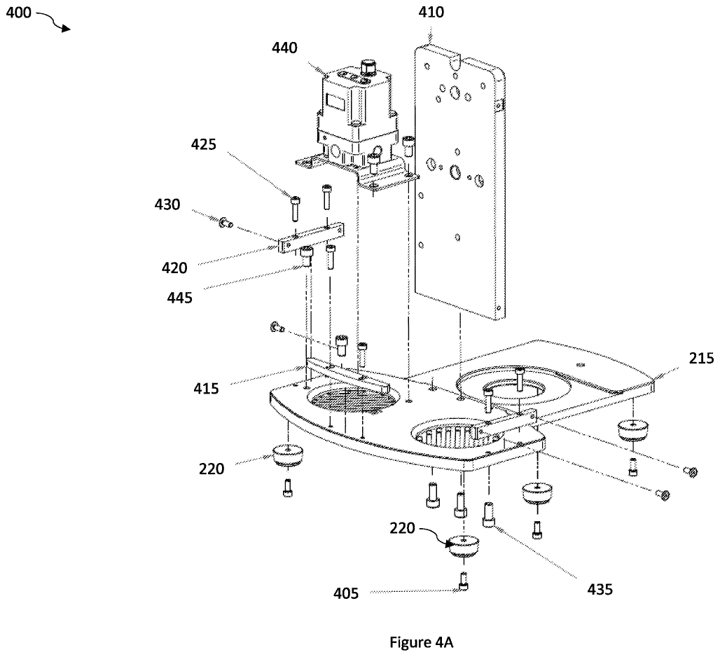

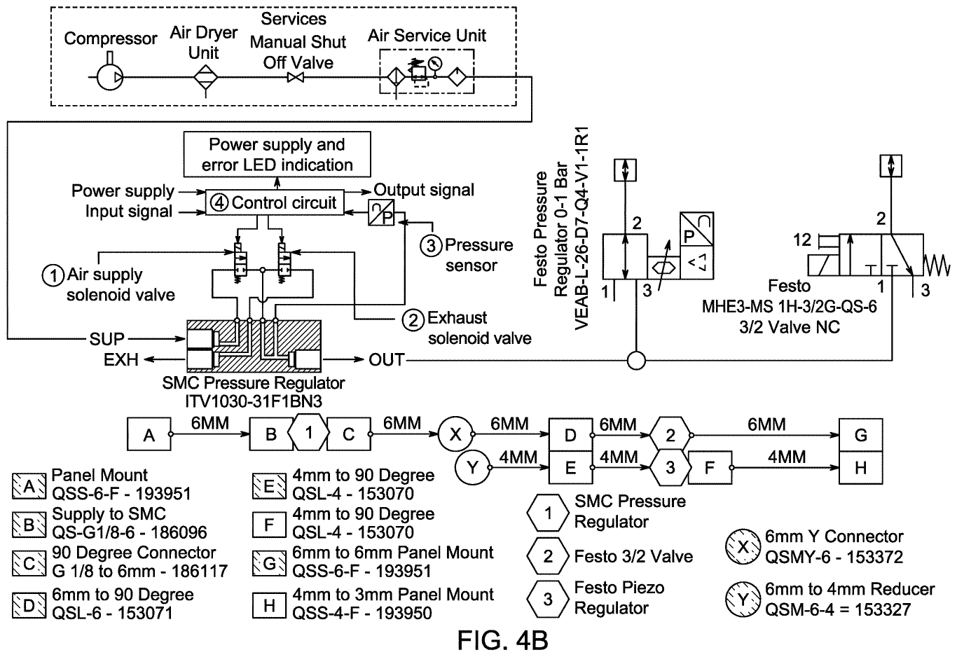

FIG. 4A is an isometric view of a CAD drawing illustrating an example embodiment of a base assembly 400 of the delivery platform 100 shown in FIG. 1. As shown in FIG. 4A, the base assembly 400 includes the base plate 215 and feet 220. Each foot 220 can be secured to the base plate 215 via a screw 405. In some embodiments, the screw 405 can include a M4×10 stainless steel screw. As further shown in FIG. 4A, the base assembly 400 includes an upright mounting spine 410. The upright mounting spine 410 can provide a base of support and a coupling mechanism for the pod nest 110, and the atomizer nest 115. The upright mounting spine 410 can be coupled to the base 215 and to the enclosure 205. The enclosure 215 can be coupled to the base assembly 400 via one or more supports. For example, the base assembly 400 includes a first rear cover support 415 and a second rear cover support 420. The second rear cover support 420 can be coupled to the base plate 215 via one or more screws 425. In some embodiments, the screws 425 can be M4×16 stainless steel screws. The enclosure 215 can be coupled to the second rear cover support 420 via one or more screws 430. In some embodiments, the screws 430 can include M4×10 ultra-low head screws. The upright mounting spine can be coupled to the base plate 215 via one or more screws 435. In some embodiments, the screws 435 can include M6×16 stainless steel screws.

As further shown in FIG. 4A, the base assembly 400 includes a pressure regulator 440. The pressure regulator 440 can be secured to the base plate 215 via one or more screws 445. The pressure regulator 440 can be coupled to the power input 135 via a circuit board. The pressure regulator 440 can be configured to control an amount of pressure of the delivery solution provided to the pod 105 via the atomizer nest 115.

The pressure regulator 440 is coupled to the fluid sources via a network of pneumatic connections, as illustrated in FIG. 4B, which includes a pneumatic diagram of some implementations of the delivery platform 100. The regulator 440 has a maximum input pressure range of 1 MPa and an output range of 0.005 to 0.5 MPa and a maximum flow rate of 200 LPM.

In some embodiments, the screws 445 can include M6×10 socket head cap screws.

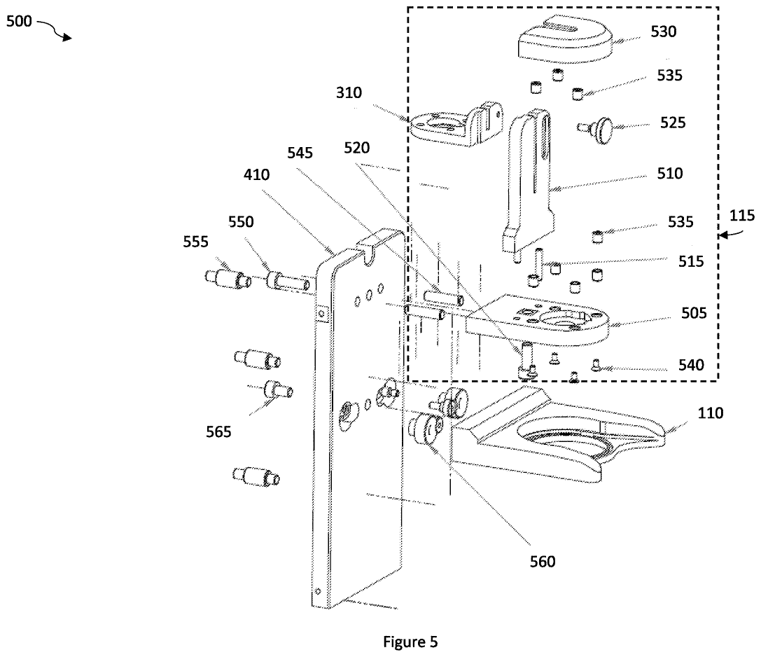

FIG. 5 is an isometric view of a CAD drawing illustrating an example embodiment of a spine assembly 500 of the delivery platform 100 shown in FIG. 1. As shown in FIG. 5, the atomizer nest 115 can be coupled to the upright mounting spine 410. The atomizer nest 115, shown within the dash-line box, includes a spray head base mounting platform 505 and a clippard module upper mount 510. A plurality of dowel pins 515 couple the clippard module upper mount 510 to the spray head base mounting platform 505. In some embodiments, the dowel pins 515 can be 4×20 mm. The clippard module upper mount 510 can further be coupled to the spray head base mounting platform 505 via a screw 520. In some embodiments, the screw can be an M6×16 socket head cap screw. The clippard module upper mount 510 can couple to the Eppendorf base support 310 via a knob 525. In some embodiments, the knob 525 can include a knurled thumb knob 525. The knob 525 can include a screw, such as a M4×10 mm screw for coupling the clippard module upper mount 510 to the Eppendorf base support 310.

As further shown in FIG. 5, the atomizer nest 115 also includes a clippard module lower mount 530. The clippard module lower mount 530 can be coupled to the spray head base mounting platform 505 via a plurality of magnets 535. In some embodiments, the magnets 530 can be 6×6 mm. The clippard module lower mount 535 can be further secured to the spray head base mounting platform 505 via a plurality of screws 540. In some embodiments, the screws 540 can include M3×6 mm flat head cap screws.

The spray head base mounting platform 505 can be coupled to the upright mounting spine 410 via a plurality of dowel pins 545. In some embodiments, the dowel pins 545 can be 6×25 mm. A screw 550 further couples the spray head base mounting platform 505 to the upright mounting spine 410. In some embodiments, the screw 550 can include a M6×20 stainless steel screw. The spine assembly 500 also includes a shaft 555. The shaft 555 can be configured for mounting the electrical and pneumatic subcomponent base plate. In some embodiments, the shaft 555 can include a rotary stepped shaft 555.

As further shown in FIG. 5, the pod nest 110 can be coupled to the upright mounting spine 410 via a plurality of bushings 560. In some embodiments, the bushings 560 can include notched-type bushings. The pod nest 120 can be configured to slide down onto the bushings 560. The pod nest 110 can be also be coupled to the upright mounting spine 410 via a screw 565. In some embodiments, the screw 565 can include a M6×10 socket head cap screw.

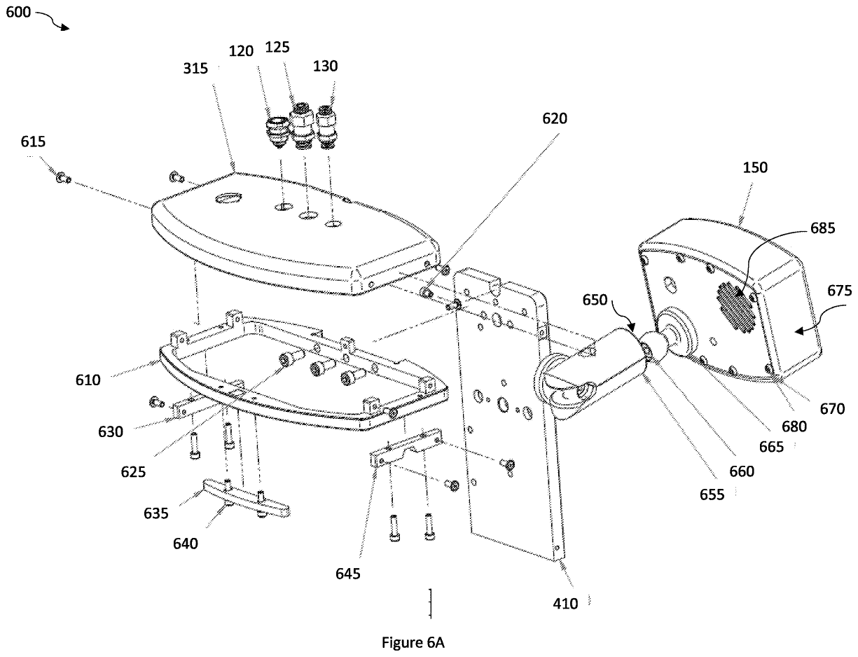

FIG. 6A is an isometric view of a CAD drawing illustrating an example embodiment of a top assembly 600 of the delivery platform 100 shown in FIG. 1. The top assembly 600 includes a top cover 315. The top cover 315 can be secured to a support rib 610 via a plurality of screws 615. In some embodiments, the screws 615 can include M4×10 ultra-low head screws. The top cover 315 can also include cutouts for the clippard valve connector 120, the sample pressure connector 125, and the air pressure connector 130. In some embodiments, the clippard valve connector 120 can include a 2 pin socket connector configured with a blue nut. In some embodiments, the sample pressure connector 125 can include a bulkhead tube fitting. In some embodiments, the air pressure connector 130 can include a push-in bulkhead connector. The top assembly 600 also includes a screw 620 configured to secure a folded section of the outer cover to the central spine 410, which is illustrated in FIG. 6B. In some embodiments, the screw 620 can include a M4×6 stainless steel screw.

As shown in FIG. 6A, the upright mounting spine 410 can be secured to the support rib 610 via a plurality of screws 625. In some embodiments, the screws 625 can be M6×16 stainless steel screws. Additionally, the top assembly 600 can include one or more supports. Support 630 can be coupled to the support rib 610 via a plurality of screws. Support 635 can be coupled to the support rib 610 via a plurality of screws 640. In some embodiments, the screws 640 can be M4×16 stainless steel screws. Support 645 can also be coupled to the support rib 610 via a plurality of screws.

As further shown in FIG. 6A, the HMI 150 can be affixed to a ball end joint assembly 650. The ball joint assembly 650 can allow the HMI 150 to be positioned in a manner suitable for viewing by an operator of the delivery platform 100. The ball end joint assembly 650 can be coupled to portions of the enclosure 205 previously described in relation to FIG. 2. The ball end joint assembly 650 can include a ball joint socket 655. The ball joint socket 655 can be coupled to a ball end joint 660. In some embodiments, the ball end join 660 can include a M8×40 stainless steel screw. The ball end joint assembly 650 also includes a joint assembly mounting plate 665, which can be coupled to the HMI mounting plate 670. The HMI mounting plate 670 can be secured to a HMI front enclosure 675 via a plurality of screws 680. In some embodiments, the screws 680 can include M4×10 button stainless steel screws. As further shown in FIG. 6A, the HMI mounting plate 670 can include a plurality of cutouts 685 to release heat generated by the display 155 and/or the circuitry of the HMI 150.

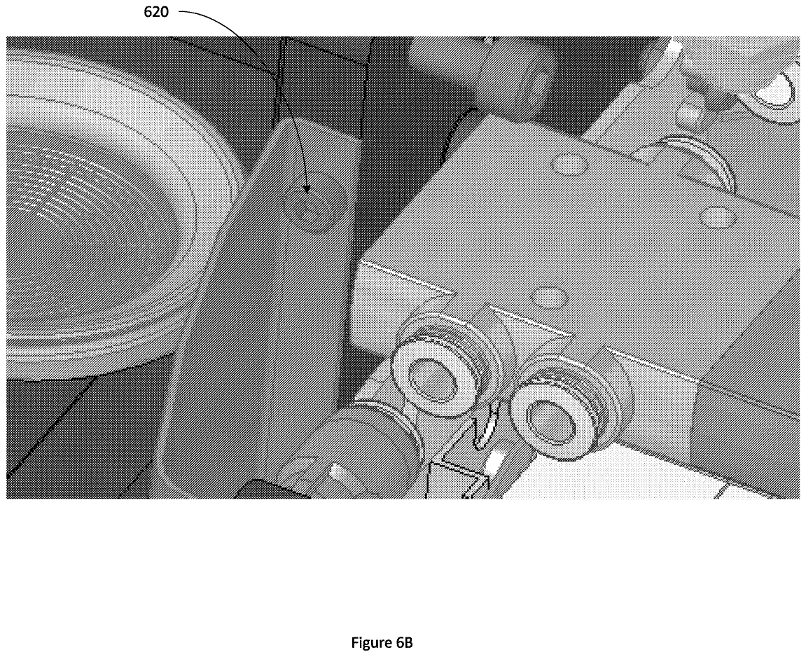

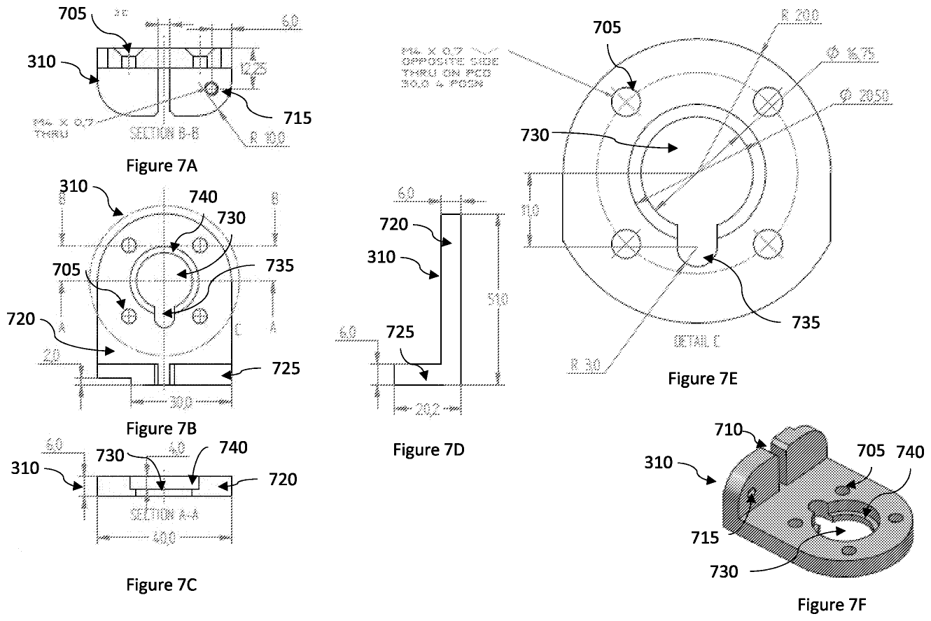

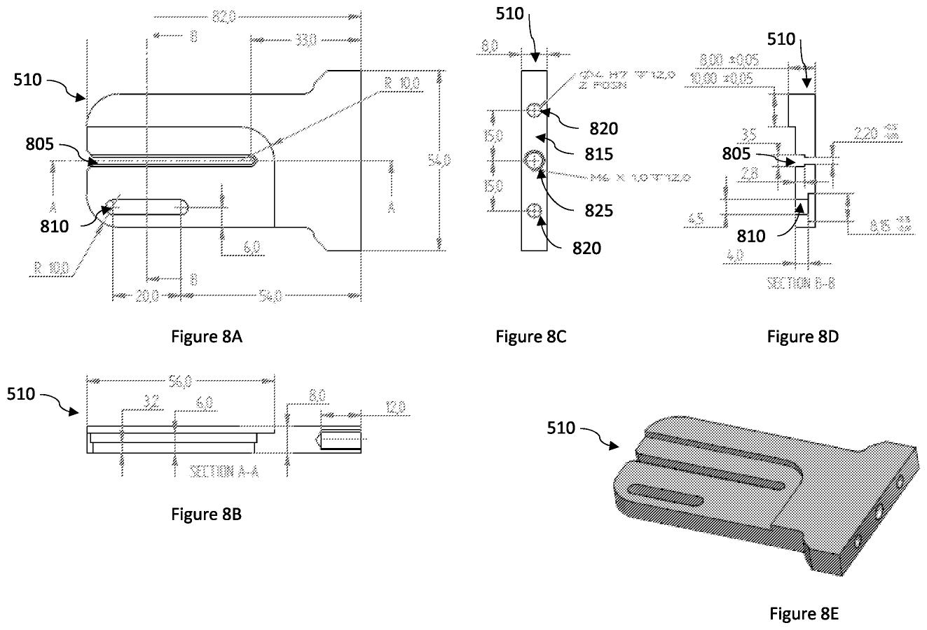

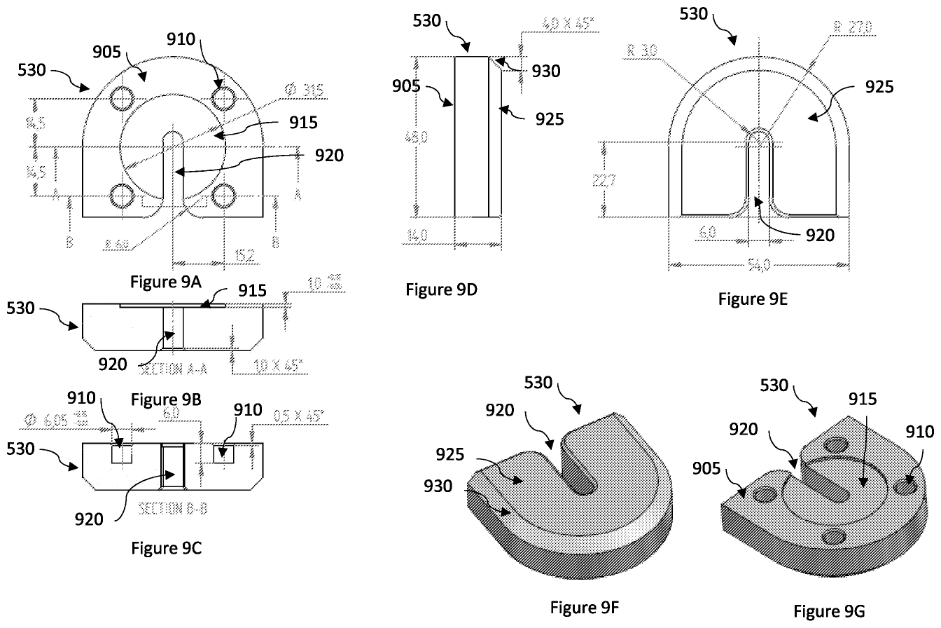

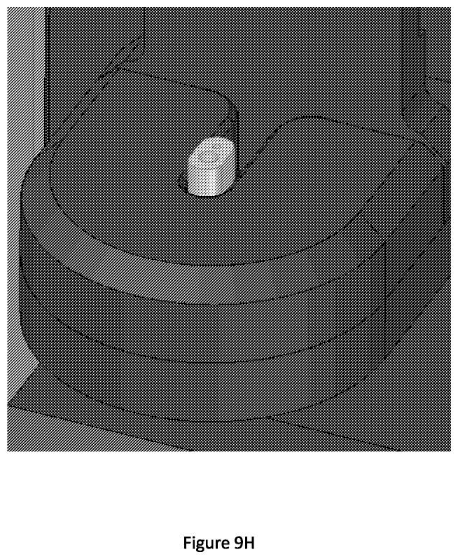

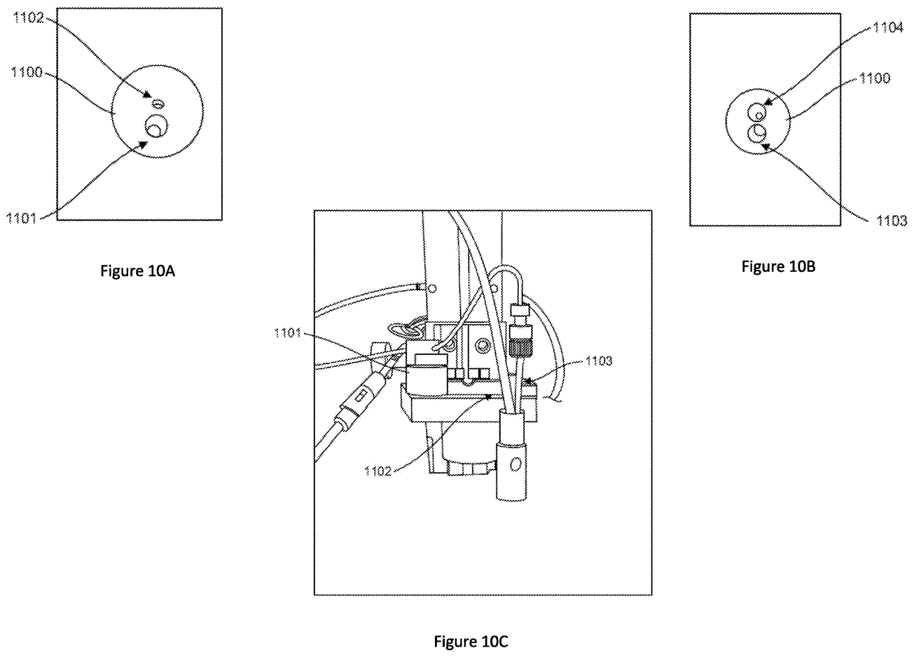

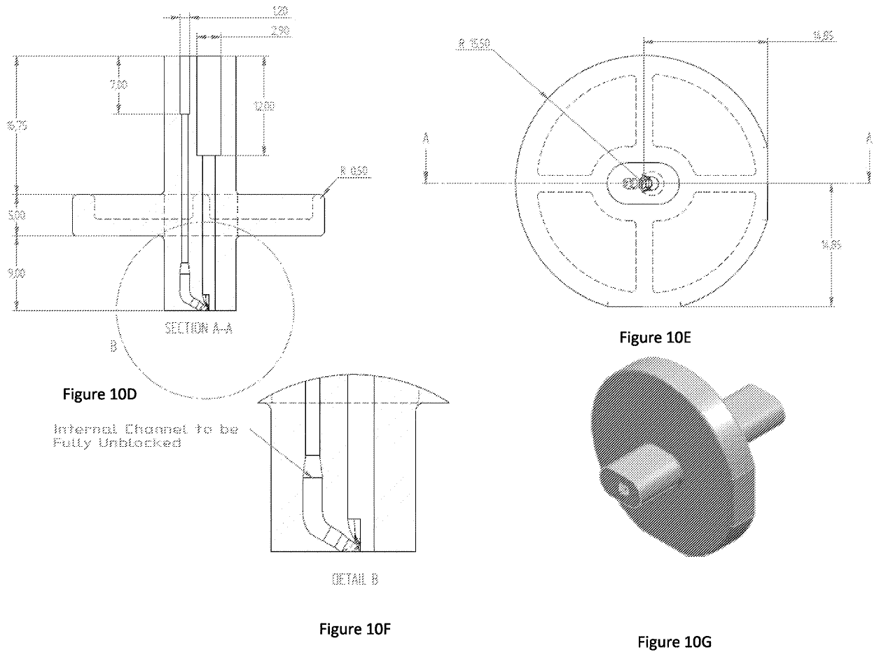

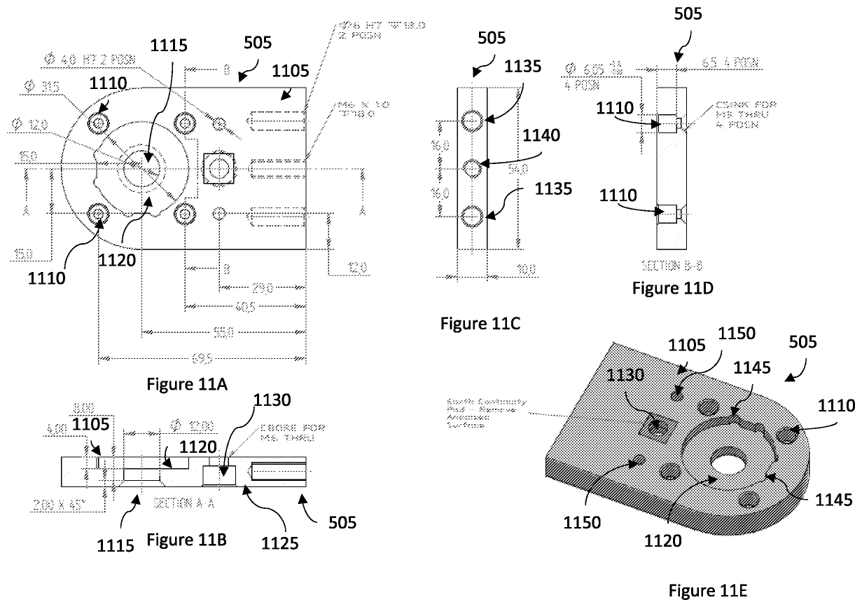

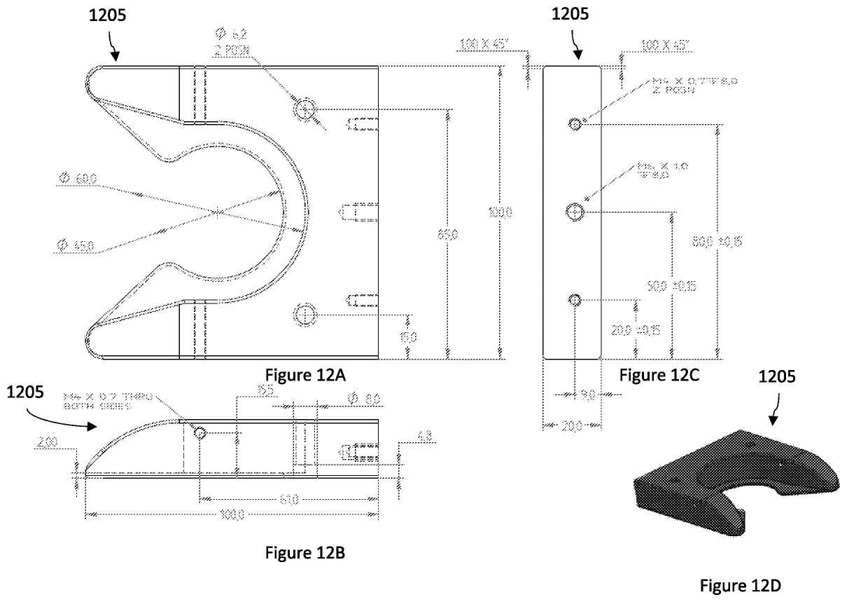

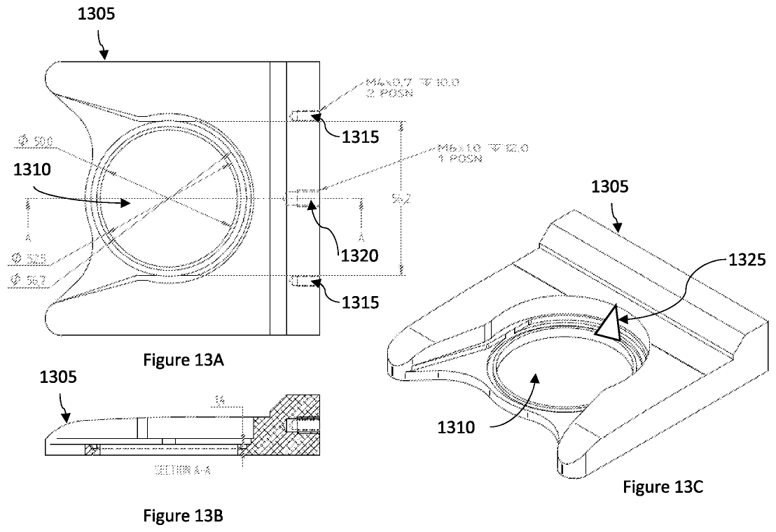

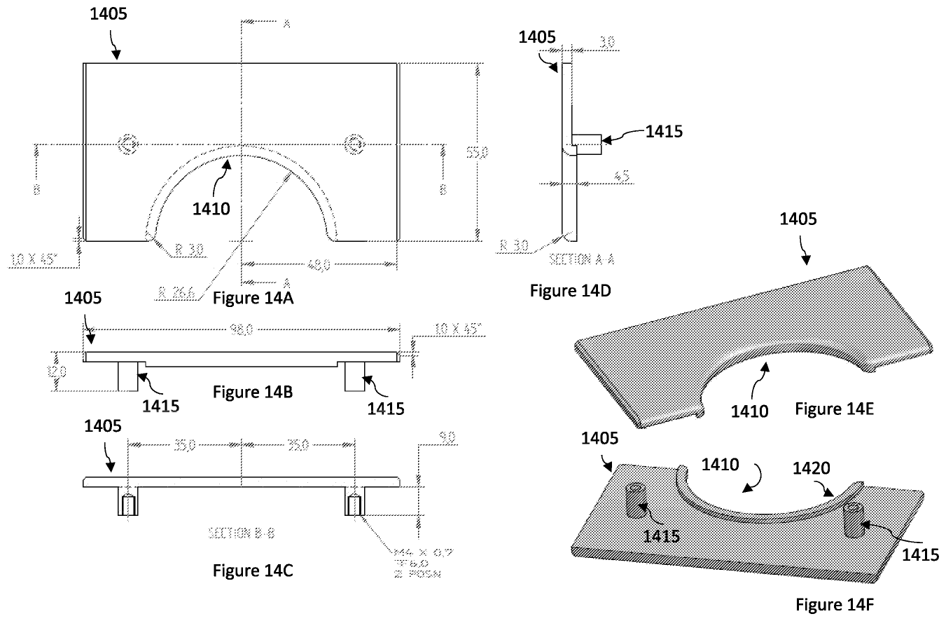

FIGS. 7A-7E are CAD drawings illustrating an example Eppendorf base support of the delivery platform 100 of FIG. 1. The Eppendorf base support shown in FIGS. 7A-7E corresponds to the Eppendorf base support 310 shown in FIGS. 3 and 5. The dimensions of the Eppendorf base 310 shown in FIGS. 7A-7E are exemplary and not intended to limit the size or configuration of the Eppendorf base support 310. The Eppendor base support 310 includes a bracket that holds the payload reservoir in space. The payload reservoir is not secured in place and a user is free to remove it from the bracket without disengaging any clamping mechanism.

FIG. 7A shows a horizontal cross-sectional view of a first end of the Eppendorf base 310. As seen in FIG. 7A, the Eppendorf base 310 includes a plurality of holes 705 and a slot 710. The holes 705 are features for employing a clamping mechanism. The slot 710 facilitates the screws that secure the Clippard Pinch Valve to the bracket 510 as part of the assembly 115 and allows the distance between the pinch valve and the atomiser to be varied.

Subsequently, the delivery solution containing the payload (e.g., cargo) is sprayed via the atomizer. The controller may control the amount and duration of the spray. For example, the delivery solution may be sprayed for about 300 ms. For spraying the delivery solution, the cargo may be introduced to the spray head via microvial or injected via resealable injection port.

After the delivery solution is sprayed, a stop solution can be introduced via a disposable tube set and/or sterile plastic needle/cannula. The stop solution may be supplied manually, or may be supplied automatically using the pump and the controller. A desired amount of stop solution is introduced into the chamber. For example, about 10 mL of stop solution may be introduced over about 20 seconds. In some implementations, no stop solution is introduced to the cells.

Following the introduction of the stop solution, the cells are resuspended. For the resuspension, about 60 mL medium, which may be a used, new medium or the medium that was previously drained from the chamber, can be introduced by a syringe or a pump. The duration for the resuspension step may be about 1 minute. To improve resuspension, various methods such as tilting of the platform, agitation (e.g., vibration of the platform), and the like may be used during the resuspension process or after the resuspension process.

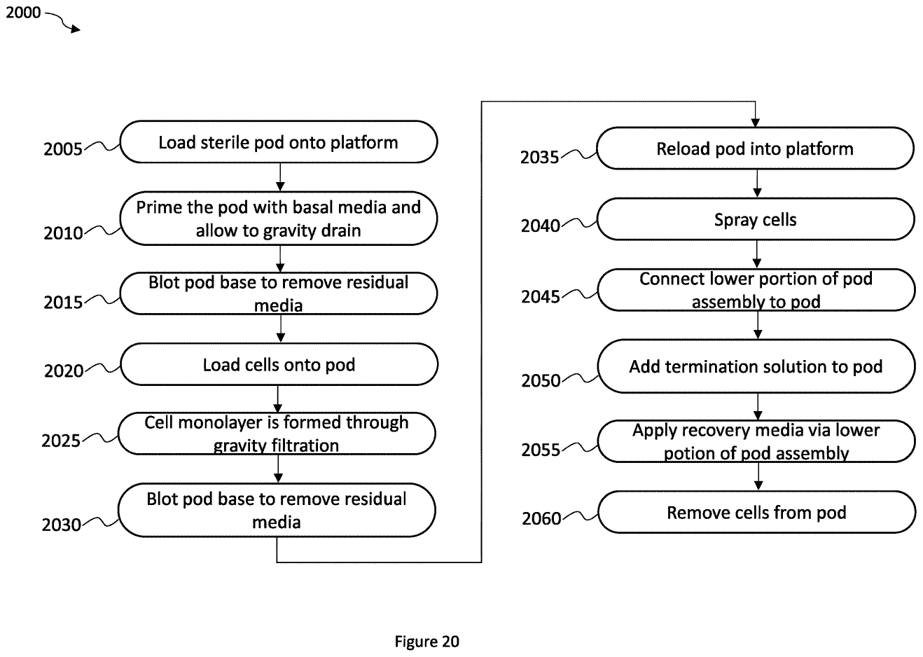

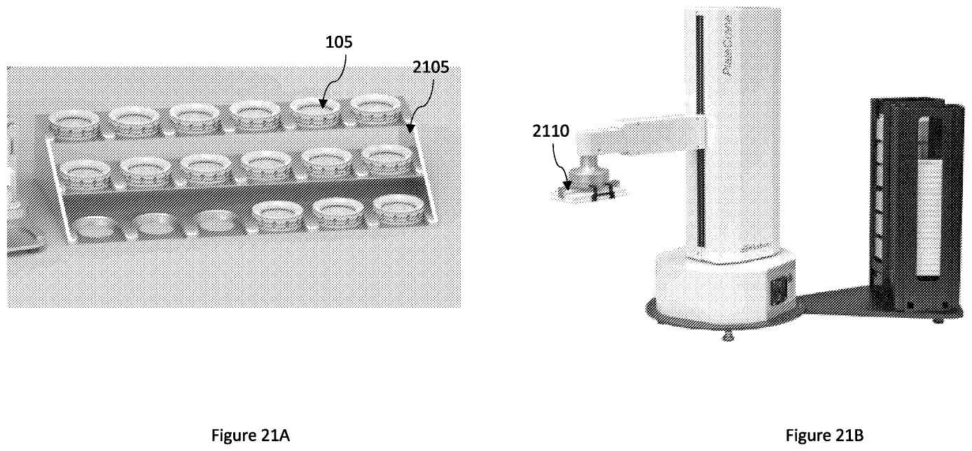

After the cells are resuspended in the medium, the engineered cells are collected for further processes. The pod may be flushed or washed after the process for subsequent procedures. Alternatively or additionally, the entire pod or a part of the pod may be made as a disposable unit that can be disposed after a use and replaced with a new one. FIG. 20 shows an exemplary process 2000. The process 2000, however, is not limited to operations shown in FIG. 20, and the process parameters, such as amount (volume) of medium, number of cells, concentration, duration for each step, may be varied depending on applications.

With reference to FIG. 20, at 2005, a sterile pod can be loaded onto the platform. At 2010, the pod can be primed with basal media and gravity can be allowed to drain the pod. At 2015, the pod base can be blotted to remove residual media. At 220 cells can be loaded onto the pod. At 2025, a cell monolayer can be formed through gravity filtration. At 2030, the pod base can be blotted to remove residual media. At 2035, the pod can be reloaded into the platform. At 2040, the cells can be sprayed by the platform with the delivery solution. At 2045, the lower portion of the pod assembly can be connected. At 2050, termination solution can be added to the pod. At 2055, recovery media can be applied via the lower portion. At 2060, cells can be removed from the pod.

The exemplary embodiments described in the Example 1 section can transfect from about 0.5 million to 15 million cells in a single transfection. The platform can allow consistent delivery of cargos, such as mRNA and the like, to T cells. The system may be enclosed within a biosafety cabinet for a sterile operation. The operation of the system may be performed manually or automatically. For the automated operation, the fluid handling system can be controlled automatically via the controller and control software. The platform may be configured as a multiple-use system which can be reused after cleaning and washing. In some implementations, the platform may be configured as a single-use, disposable system which includes disposable parts such as a disposable pod.

### Embodiments of Example Delivery Protocols

The invention is based on the surprising discovery that compounds or mixtures of compounds (compositions) are delivered into the cytoplasm of eukaryotic cells by contacting the cells with a solution containing a compound(s) to be delivered (e.g., payload). Preferably, the solution is delivered to the cells in the form of a spray, e.g., aqueous particles. (see, e.g., PCT/US2015/057247 and PCT/IB2016/001895, hereby incorporated in their entirety by reference). For example, the cells are coated with the spray but not soaked or submersed in the delivery compound-containing solution. In some implementations, the delivery solution can include an agent that permeabilizes or dissolves a cell membrane, although the agent may not be required to affect delivery of the payload the agent may enhance delivery. Exemplary agents that permeate or dissolve a eukaryotic cell membrane include alcohols and detergents such as ethanol and Triton X-100, respectively. Other exemplary detergents, e.g., surfactants include polysorbate 20 (e.g., Tween 20), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), sodium dodecyl sulfate (SDS), and octyl glucoside.

An example of conditions to achieve a coating of a population of coated cells include delivery of a fine particle spray, e.g., the conditions exclude dropping or pipetting a bolus volume of solution on the cells such that a substantial population of the cells are soaked or submerged by the volume of fluid. Thus, the mist or spray comprises a ratio of volume of fluid to cell volume. Alternatively, the conditions comprise a ratio of volume of mist or spray to exposed cell area, e.g., area of cell membrane that is exposed when the cells exist as a confluent or substantially confluent layer on a substantially flat surface such as the bottom of a tissue culture vessel, e.g., a well of a tissue culture plate, e.g., a microtiter tissue culture plate.

“Cargo” or “payload” are terms used to describe a compound, or composition that is delivered via an aqueous solution across a cell plasma membrane and into the interior of a cell.

In an aspect, delivering a payload across a plasma membrane of a cell includes providing a population of cells and contacting the population of cells with a volume of an aqueous solution. The aqueous solution includes the payload. In some implementations, the aqueous solution includes no alcohol. In some implementations, the aqueous solution includes an alcohol content greater than 0.2 percent concentration. In some implementations, the aqueous solution includes the payload and an alcohol content greater than 5 percent concentration. The volume of the aqueous solution may be a function of exposed surface area of the population of cells, or may be a function of a number of cells in the population of cells.

In another aspect, a composition for delivering a payload across a plasma membrane of a cell includes an aqueous solution including the payload, greater than 46 mM salt, less than 121 mM sugar, and less than 19 mM buffering agent. In some implementations, the aqueous solution does not include any alcohol. In some implementations, the aqueous solution includes alcohol at greater than 0.2 percent concentration. For example, the alcohol, e.g., ethanol, concentration is greater than 2 percent, greater than 5 percent, and/or does not exceed 50%.

One or more of the following features can be included in any feasible combination. The volume of solution to be delivered to the cells is a plurality of units, e.g., a spray, e.g., a plurality of droplets on aqueous particles. The volume is described relative to an individual cell or relative to the exposed surface area of a confluent or substantially confluent (e.g., at least 75%, at least 80% confluent, e.g., 85%, 90%, 95%, 97%, 98%, 100%) cell population. For example, the volume can be between 6.0×10⁻⁷ microliter per cell and 7.4×10⁻⁴ microliter per cell. The volume is between 4.9×10⁻⁶ microliter per cell and 2.2×10⁻³ microliter per cell. The volume can be between 9.3×10⁻⁶ microliter per cell and 2.8×10⁻⁵ microliter per cell. The volume can be about 1.9×10⁻⁵ microliters per cell, and about is within 10 percent. The volume is between 6.0×10⁻⁷ microliter per cell and 2.2×10⁻³ microliter per cell. The volume can be between 2.6×10⁻⁹ microliter per square micrometer of exposed surface area and 1.1×10⁻⁶ microliter per square micrometer of exposed surface area. The volume can be between 5.3×10⁻⁸ microliter per square micrometer of exposed surface area and 1.6×10⁻⁷ microliter per square micrometer of exposed surface area. The volume can be about 1.1×10⁻⁷ microliter per square micrometer of exposed surface area. About can be within 10 percent.

Confluency of cells refers to cells in contact with one another on a surface. For example, it can be expressed as an estimated (or counted) percentage, e.g., 10% confluency means that 10% of the surface, e.g., of a tissue culture vessel, is covered with cells, 100% means that it is entirely covered. For example, adherent cells grow two dimensionally on the surface of a tissue culture well, plate or flask. Non-adherent cells can be spun down, pulled down by a vacuum, or tissue culture medium aspiration off the top of the cell population, or removed by aspiration or vacuum removal from the bottom of the vessel.

Contacting the population of cells with the volume of aqueous solution can be performed by gas propelling the aqueous solution to form a spray. The gas can include nitrogen, ambient air, or an inert gas. The spray can include discrete units of volume ranging in size from, 1 nm to 100 μm, e.g., 30-100 μm in diameter. The spray includes discrete units of volume with a diameter of about 30-50 μm. A total volume of aqueous solution of 20 μl can be delivered in a spray to a cell-occupied area of about 1.9 cm², e.g., one well of a 24-well culture plate. A total volume of aqueous solution of 10 μl is delivered to a cell-occupied area of about 0.95 cm², e.g., one well of a 48-well culture plate. Typically, the aqueous solution includes a payload to be delivered across a cell membrane and into cell, and the second volume is a buffer or culture medium (e.g., a stop solution) that does not contain the payload. Alternatively, the second volume (buffer or media) can also contain payload. In some embodiments, the aqueous solution includes a payload and an alcohol, and the second volume does not contain alcohol (and optionally does not contain payload). The population of cells can be in contact with said aqueous solution for 0.1 10 minutes prior to adding a second volume of buffer or culture medium to submerse or suspend said population of cells. The buffer or culture medium can be phosphate buffered saline (PBS). The population of cells can be in contact with the aqueous solution for 2 seconds to 5 minutes prior to adding a second volume of buffer or culture medium to submerse or suspend the population of cells. The population of cells can be in contact with the aqueous solution, e.g., containing the payload, for 30 seconds to 2 minutes prior to adding a second volume of buffer or culture medium, e.g., without the payload, to submerse or suspend the population of cells. The population of cells can be in contact with a spray for about 1-2 minutes prior to adding the second volume of buffer or culture medium to submerse or suspend the population of cells. During the time between spraying of cells and addition of buffer or culture medium, the cells remain hydrated by the layer of moisture from the spray volume.

The aqueous solution can include an ethanol concentration of 5 to 30%. The aqueous solution can include one or more of 75 to 98% H₂O, 2 to 45% ethanol, 6 to 91 mM sucrose, 2 to 500 mM KCl, 2 to 35 mM ammonium acetate, and 1 to 14 mM (4-(2-hydroxyethyl)-1-piperazineethanesulfonic acid) (HEPES). For example, the delivery solution contains 10⁶ mM KCl and 27% ethanol.

The population of cells can include adherent cells or non-adherent cells. The adherent cells can include at least one of primary mesenchymal stem cells, fibroblasts, monocytes, macrophages, lung cells, neuronal cells, fibroblasts, human umbilical vein (HUVEC) cells, Chinese hamster ovary (CHO) cells, and human embryonic kidney (HEK) cells or immortalized cells, such as cell lines. In preferred embodiments, the population of cells comprises non-adherent cells, e.g., the % non-adherent cells in the population is at least 50%, 60%, 75%, 80%, 90%, 95%, 98%, 99% or 100% non-adherent cells. Non-adherent cells primary cells as well as immortalized cells (e.g., cells of a cell line). Exemplary non-adherent/suspension cells include primary hematopoietic stem cell (HSC), T cells (e.g., CD3+ cells, CD4+ cells, CD8+ cells), natural killer (NK) cells, cytokine-induced killer (CIK) cells, human cord blood CD34+ cells, B cells, or cell lines such as Jurkat T cell line.

The payload can include a small chemical molecule, a peptide or protein, or a nucleic acid. The small chemical molecule can be less than 1,000 Da. The chemical molecule can include MitoTracker® Red CMXRos, propidium iodide, methotrexate, and/or DAPI (4′,6-diamidino-2-phenylindole). The peptide can be about 5,000 Da. The peptide can include ecallantide under trade name Kalbitor, is a 60 amino acid polypeptide for the treatment of hereditary angioedema and in prevention of blood loss in cardiothoracic surgery), Liraglutide (marketed as the brand name Victoza, is used for the treatment of type II diabetes, and Saxenda for the treatment of obesity), and Icatibant (trade name Firazyer, a peptidomimetic for the treatment of acute attacks of hereditary angioedema). The small-interfering ribonucleic acid (siRNA) molecule can be about 20-25 base pairs in length, or can be about 10,000-15,000 Da. The siRNA molecule can reduces the expression of any gene product, e.g., knockdown of gene expression of clinically relevant target genes or of model genes, e.g., glyceraldehyde-3phosphate dehydrogenase (GAPDH) siRNA, GAPDH siRNA-FITC, cyclophilin B siRNA, and/or lamin siRNA. Protein therapeutics can include peptides, enzymes, structural proteins, receptors, cellular proteins, or circulating proteins, or fragments thereof. The protein or polypeptide be about 100-500,000 Da, e.g., 1,000-150,000 Da. The protein can include any therapeutic, diagnostic, or research protein or peptide, e.g., beta-lactoglobulin, ovalbumin, bovine serum albumin (BSA), and/or horseradish peroxidase. In other examples, the protein can include a cancer-specific apoptotic protein, e.g., Tumor necrosis factor-related apoptosis inducing protein (TRAIL).

An antibody is generally about 150,000 Da in molecular mass. The antibody can include an anti-actin antibody, an anti-GAPDH antibody, an anti-Src antibody, an anti-Myc ab, and/or an anti-Raf antibody. The antibody can include a green fluorescent protein (GFP) plasmid, a GLuc plasmid and, and a BATEM plasmid. The DNA molecule can be greater than 5,000,000 Da. In some examples, the antibody can be a murine-derived monoclonal antibody, e.g., ibritumomab tiuxetin, muromomab-CD3, tositumomab, a human antibody, or a humanized mouse (or other species of origin) antibody. In other examples, the antibody can be a chimeric monoclonal antibody, e.g., abciximab, basiliximab, cetuximab, infliximab, or rituximab. In still other examples, the antibody can be a humanized monoclonal antibody, e.g., alemtuzamab, bevacizumab, certolizumab pegol, daclizumab, gentuzumab ozogamicin, trastuzumab, tocilizumab, ipilimumamb, or panitumumab. The antibody can comprise an antibody fragment, e.g., abatecept, aflibercept, alefacept, or etanercept. The invention encompasses not only an intact monoclonal antibody, but also an immunologically-active antibody fragment, e.g., a Fab or (Fab)2 fragment; an engineered single chain Fv molecule; or a chimeric molecule, e.g., an antibody which contains the binding specificity of one antibody, e.g., of murine origin, and the remaining portions of another antibody, e.g., of human origin. The payload can include a therapeutic agent. A therapeutic agent, e.g., a drug, or an active agent”, can mean any compound useful for therapeutic or diagnostic purposes, the term can be understood to mean any compound that is administered to a patient for the treatment of a condition. Accordingly, a therapeutic agent can include, proteins, peptides, antibodies, antibody fragments, and small molecules. Therapeutic agents described in U.S. Pat. No.7,667,004 (incorporated herein by reference) can be used in the methods described herein. The therapeutic agent can include at least one of cisplatin, aspirin, statins (e.g., pitavastatin, atorvastatin, lovastatin, pravastatin, rosuvastatin, simvastatin, promazine HCl, chloropromazine HCl, thioridazine HCl, Polymyxin B sulfate, chloroxine, benfluorex HCl and phenazopyridine HCl), and fluoxetine. The payload can include a diagnostic agent. The diagnostic agent can include a detectable label or marker such as at least one of methylene blue, patent blue V, and indocyanine green. The payload can include a fluorescent molecule. The payload can include a detectable nanoparticle. The nanoparticle can include a quantum dot.

The population of non-adherent cells can be substantially confluent, such as greater than 75 percent confluent. Confluency of cells refers to cells in contact with one another on a surface. For example, it can be expressed as an estimated (or counted) percentage, e.g., 10% confluency means that 10% of the surface, e.g., of a tissue culture vessel, is covered with cells, 100% means that it is entirely covered. For example, adherent cells grow two dimensionally on the surface of a tissue culture well, plate or flask. Non-adherent cells can be spun down, pulled down by a vacuum, or tissue culture medium aspiration off the top of the cell population, or removed by aspiration or vacuum removal from the bottom of the vessel. The population of cells can form a monolayer of cells.

The alcohol can be selected from methanol, ethanol, isopropyl alcohol, butanol and benzyl alcohol. The salt can be selected from NaCl, KCl, Na₂HPO₄, KH₂PO₄, and C₂H₃O₂NH. In preferred embodiments, the salt is KCl. The sugar can include sucrose. The buffering agent can include 4-2-(hydroxyethyl)-1-piperazineethanesulfonic acid.

The present subject matter relates to a method for delivering molecules across a plasma membrane. The present subject matter finds utility in the field of intra-cellular delivery, and has application in, for example, delivery of molecular biological and pharmacological therapeutic agents to a target site, such as a cell, tissue, or organ. The method of the present subject matter comprises introducing the molecule to an aqueous composition to form a matrix; atomizing the matrix into a spray; and contacting the matrix with a plasma membrane.

This present subject matter relates to a composition for use in delivering molecules across a plasma membrane. The present subject matter finds utility in the field of intra-cellular delivery, and has application in, for example, delivery of molecular biological and pharmacological therapeutic agents to a target site, such as a cell, tissue, or organ. The composition of the present subject matter comprises an alcohol; a salt; a sugar; and/or a buffering agent.

In some implementations, demonstrated is a delivery technique that facilitates intracellular delivery of molecules independent of the molecule and cell type. Nanoparticles, small molecules, nucleic acids, proteins and other molecules can be efficiently delivered into suspension cells or adherent cells in situ, including primary cells and stem cells, with low cell toxicity and the technique is compatible with high throughput and automated cell-based assays. Some example methods described herein include a payload, wherein the payload includes an alcohol. By the term “an alcohol” is meant a polyatomic organic compound including a hydroxyl (—OH) functional group attached to at least one carbon atom. The alcohol may be a monohydric alcohol and may include at least one carbon atom, for example methanol. The alcohol may include at least two carbon atoms (e.g. ethanol). In other aspects, the alcohol comprises at least three carbons (e.g. isopropyl alcohol). The alcohol may include at least four carbon atoms (e.g., butanol), or at least seven carbon atoms (e.g., benzyl alcohol). The example payload may include no more than 50% (v/v) of the alcohol, more preferably, the payload comprises 2-45% (v/v) of the alcohol, 5-40% of the alcohol, and 10-40% of the alcohol. The payload may include 20-30% (v/v) of the alcohol.

In some implementations, the payload delivery solution includes 25% (v/v) of the alcohol. Alternatively, the payload can include 2-8% (v/v) of the alcohol, or 2% of the alcohol. The alcohol may include ethanol and the payload comprises 5, 10, 20, 25, 30, and up to 40% or 50% (v/v) of ethanol, e.g., 27%. Example methods may include methanol as the alcohol, and the payload may include 5, 10, 20, 25, 30, or 40% (v/v) of the methanol. The payload may include 2-45% (v/v) of methanol, 20-30% (v/v), or 25% (v/v) methanol. Preferably, the payload includes 20-30% (v/v) of methanol. Further alternatively, the alcohol is butanol and the payload comprises 2, 4, or 8% (v/v) of the butanol.

In some aspects of the present subject matter, the payload is in an isotonic solution or buffer.

According to the present subject matter, the payload may include at least one salt. The salt may be selected from NaCl, KCl, Na₂HPO₄, C₂H₃O₂NH₄ and KH₂PO₄. For example, KCl concentration ranges from 2 mM to 500 mM. In some preferred embodiments, the concentration is greater than 100 mM, e.g., 106 mM.

According to example methods of the present subject matter, the payload may include a sugar (e.g., a sucrose, or a disaccharide). According to example methods, the payload comprises less than 121 mM sugar, 6-91 mM, or 26-39 mM sugar. Still further, the payload includes 32 mM sugar (e.g., sucrose). Optionally, the sugar is sucrose and the payload comprises 6.4, 12.8, 19.2, 25.6, 32, 64, 76.8, or 89.6 mM sucrose.

According to example methods of the present subject matter, the payload may include a buffering agent (e.g. a weak acid or a weak base). The buffering agent may include a zwitterion. According to example methods, the buffering agent is 4-(2-hydroxyethyl)-1-piperazineethanesulfonic acid. The payload may comprise less than 19 mM buffering agent (e.g., 1-15 mM, or 4-6 mM or 5 mM buffering agent). According to example methods, the buffering agent is 4-(2-hydroxyethyl)-1-piperazineethanesulfonic acid and the payload comprises 1, 2, 3, 4, 5, 10, 12, 14 mM 4-(2-hydroxyethyl)-1-piperazineethanesulfonic acid. Further preferably, the payload comprises 5 mM 4-(2-hydroxyethyl)-1-piperazineethanesulfonic acid.

According to example methods of the present subject matter, the payload includes ammonium acetate. The payload may include less than 46 mM ammonium acetate (e.g., between 2-35 mM, 10-15 mM, ore 12 mM ammonium acetate). The payload may include 2.4, 4.8, 7.2, 9.6, 12, 24, 28.8, or 33.6 mM ammonium acetate.

The volume of aqueous solution performed by gas propelling the aqueous solution may include compressed air (e.g. ambient air), other implementations may include inert gases, for example, helium, neon, and argon.

In certain aspects of the present subject matter, the population of cells may include adherent cells (e.g., lung, kidney, immune cells such as macrophages) or non-adherent cells (e.g., suspension cells).

In certain aspects of the present subject matter, the population of cells may be substantially confluent, and substantially may include greater than 75 percent confluent. In preferred implementations, the population of cells may form a single monolayer.

According to example methods, the payload to be delivered has an average molecular weight of up to 20,000,000 Da. In some examples, the payload to be delivered can have an average molecular weight of up to 2,000,000 Da. In some implementations, the payload to be delivered may have an average molecular weight of up to 150,000 Da. In further implementations, the payload to be delivered has an average molecular weight of up to 15,000 Da, 5,000 Da or 1,000 Da.

The payload to be delivered across the plasma membrane of a cell may include a small chemical molecule, a peptide or protein, a polysaccharide or a nucleic acid or a nanoparticle. A small chemical molecule may be less than 1,000 Da, peptides may have molecular weights about 5,000 Da, siRNA may have molecular weights around 15,000 Da, antibodies may have molecular weights of about 150,000 Da and DNA may have molecular weights of greater than or equal to 5,000,000 Da. In preferred embodiments, the payload comprises mRNA.

According to example methods, the payload includes 3.0-150.0 μM of a molecule to be delivered, more preferably, 6.6-150.0 μM molecule to be delivered (e.g. 3.0, 3.3 , 6.6, or 150.0 μM molecule to be delivered). In some implementations, the payload to be delivered has an average molecular weight of up to 15,000 Da, and the payload includes 3.3 μM molecules to be delivered.

According to example methods, the payload to be delivered has an average molecular weight of up to 15,000 Da, and the payload includes 6.6 μM to be delivered. In some implementations, the payload to be delivered has an average molecular weight of up to 1,000 Da, and the payload includes 150.0 μM to be delivered.

According to further aspects of the present subject matter, a method for delivering molecules of more than one molecular weight across a plasma membrane is provided; the method including the steps of: introducing the molecules of more than one molecular weight to an aqueous solution; and contacting the aqueous solution with a plasma membrane.

In some implementations, the method includes introducing a first molecule having a first molecular weight and a second molecule having a second molecular weight to the payload, wherein the first and second molecules may have different molecular weights, or wherein, the first and second molecules may have the same molecular weights. According to example methods, the first and second molecules may be different molecules.

In some implementations, the payload to be delivered may include a therapeutic agent, or a diagnostic agent, including, for example, cisplatin, aspirin, various statins (e.g., pitavastatin, atorvastatin, lovastatin, pravastatin, rosuvastatin, simvastatin, promazine HCl, chloropromazine HCl, thioridazine HCl, Polymyxin B sulfate, chloroxine, benfluorex HCl and phenazopyridine HCl), and fluoxetine. Other therapeutic agents include antimicrobials (aminoclyclosides (e.g. gentamicin, neomycin, streptomycin), penicillins (e.g., amoxicillin, ampicillin), glycopeptides (e.g., avoparcin, vancomycin), macrolides (e.g., erythromycin, tilmicosin, tylosin), quinolones (e.g., sarafloxacin, enrofloxin), streptogramins (e.g., viginiamycin, quinupristin-dalfoprisitin), carbapenems, lipopeptides, oxazolidinones, cycloserine, ethambutol, ethionamide, isoniazrid, para-aminosalicyclic acid, and pyrazinamide). In some examples, an anti-viral (e.g., Abacavir, Aciclovir, Enfuvirtide, Entecavir, Nelfinavir, Nevirapine, Nexavir, Oseltamivir Raltegravir, Ritonavir, Stavudine, and Valaciclovir). The therapeutic may include a protein-based therapy for the treatment of various diseases, e.g., cancer, infectious diseases, hemophilia, anemia, multiple sclerosis, and hepatitis B or C.

Additional exemplary payloads can also include detectable markers or labels such as methylene blue, Patent blue V, and Indocyanine green.

The methods described herein may also include the payload including of a detectable moiety, or a detectable nanoparticle (e.g., a quantum dot). The detectable moiety may include a fluorescent molecule or a radioactive agent (e.g., ¹²⁵I). When the fluorescent molecule is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, p-phthaldehyde and fluorescamine. The molecule can also be detectably labeled using fluorescence emitting metals such as ¹⁵²Eu, or others of the lanthanide series. These metals can be attached to the molecule using such metal chelating groups as diethylenetriaminepentacetic acid (DTPA) or ethylenediaminetetraacetic acid (EDTA). The molecule also can be detectably labeled by coupling it to a chemiluminescent compound. The presence of the chemiluminescent-tagged molecule is then determined by detecting the presence of luminescence that arises during the course of chemical reaction. Examples of particularly useful chemiluminescent labeling compounds are luminol, isoluminol, theromatic acridinium ester, imidazole, acridinium salt and oxalate ester.

In additional embodiments, the payload to be delivered may include a composition that edits genomic DNA (i.e., gene editing tools). For example, the gene editing composition may include a compound or complex that cleaves, nicks, splices, rearranges, translocates, recombines, or otherwise alters genomic DNA. Alternatively or in addition, a gene editing composition may include a compound that (i) may be included a gene-editing complex that cleaves, nicks, splices, rearranges, translocates, recombines, or otherwise alters genomic DNA; or (ii) may be processed or altered to be a compound that is included in a gene-editing complex that cleaves, nicks, splices, rearranges, translocates, recombines, or otherwise alters genomic DNA. In various embodiments, the gene editing composition comprises one or more of (a) gene editing protein; (b) RNA molecule; and/or (c) ribonucleoprotein (RNP).

In some embodiments, the gene editing composition comprises a gene editing protein, and the gene editing protein is a zinc finger nuclease (ZFN), a transcription activator-like effector nuclease (TALEN), a Cas protein, a Cre recombinase, a Hin recombinase, or a Flp recombinase. In additional embodiments, the gene editing protein may be a fusion proteins that combine homing endonucleases with the modular DNA binding domains of TALENs (megaTAL). For example, megaTAL may be delivered as a protein or alternatively, a mRNA encoding a megaTAL protein is delivered to the cells.

In various embodiments, the gene editing composition comprises a RNA molecule, and the RNA molecule comprises a sgRNA, a crRNA, and/or a tracrRNA.

In certain embodiments, the gene editing composition comprises a RNP, and the RNP comprises a Cas protein and a sgRNA or a crRNA and a tracrRNA. Aspects of the present subject matter are particularly useful for controlling when and for how long a particular gene-editing compound is present in a cell.

In various implementations of the present subject matter, the gene editing composition is detectable in a population of cells, or the progeny thereof, for (a) about 0.5, 1, 2, 3, 4, 5, 6, 7, 8, 9, 10, 12, 24, 48, 60, 72, 0.5-2, 0.5-6, 6-12 or 0.5-72 hours after the population of cells is contacted with the aqueous solution, or (b) less than about 0.5, 1, 2, 3, 4, 5, 6, 7, 8, 9, 10, 12, 24, 48, 60, 72, 0.5-2, 0.5-6, 6-12 or 0.5-72 hours after the population of cells is contacted with the aqueous solution.

In some embodiments, the genome of cells in the population of cells, or the progeny thereof, comprises at least one site-specific recombination site for the Cre recombinase, Hin recombinase, or Flp recombinase.

Aspects of the present invention relate to cells that comprise one gene editing compound, and inserting another gene editing compound into the cells. For example, one component of an RNP could be introduced into cells that express or otherwise already contain another component of the RNP. For example, cells in a population of cells, or the progeny thereof, may comprise a sgRNA, a crRNA, and/or a tracrRNA. In some embodiments the population of cells, or the progeny thereof, expresses the sgRNA, crRNA, and/or tracrRNA. Alternatively or in addition, cells in a population of cells, or the progeny thereof, express a Cas protein.

Various implementations of the subject matter herein include a Cas protein. In some embodiments, the Cas protein is a Cas9 protein or a mutant thereof. Exemplary Cas proteins (including Cas9 and non-limiting examples of Cas9 mutants) are described herein.

In various aspects, the concentration of Cas9 protein may range from about 0.1 to about 25 μg. For example, the concentration of Cas9 may be about 1 μg, about 5 μg, about 10 μg, about 15 μg, or about 20 μg. Alternatively, the concentration of Cas9 may range from about 10 ng/μL to about 300 ng/μL; for example from about 10 ng/μL to about 200 ng/μL; or from about 10 ng/μL to about 100 ng/μl, or from about 10 ng/μL to about 50 ng/μl.

In certain embodiments, the gene editing composition comprises (a) a first sgRNA molecule and a second sgRNA molecule, wherein the nucleic acid sequence of the first sgRNA molecule is different from the nucleic acid sequence of the second sgRNA molecule; (b) a first RNP comprising a first sgRNA and a second RNP comprising a second sgRNA, wherein the nucleic acid sequence of the first sgRNA molecule is different from the nucleic acid sequence of the second sgRNA molecule; (c) a first crRNA molecule and a second crRNA molecule, wherein the nucleic acid sequence of the first crRNA molecule is different from the nucleic acid sequence of the second crRNA molecule; (d) a first crRNA molecule and a second crRNA molecule, wherein the nucleic acid sequence of the first crRNA molecule is different from the nucleic acid sequence of the second crRNA molecule, and further comprising a tracrRNA molecule; or (e) a first RNP comprising a first crRNA and a tracrRNA and a second RNP comprising a second crRNA and a tracrRNA, wherein the nucleic acid sequence of the first crRNA molecule is different from the nucleic acid sequence of the second crRNA molecule.

In aspects, the ratio of the Cas9 protein to guide RNA may be 1:1, 1:2, 1:3, 1:4, 1:5, 1:6, 1:7, 1:8, 1:9, or 1:10.

In embodiments, increasing the number of times that cells go through the delivery process (alternatively, increasing the number of doses), may increase the percentage edit; wherein, in some embodiments the number of doses may include 1, 2, 3, 4, 5, 6, 7, 8, 9, or 10 doses.

In various embodiments, the first and second sgRNA or first and second crRNA molecules together comprise nucleic acid sequences complementary to target sequences flanking a gene, an exon, an intron, an extrachromosomal sequence, or a genomic nucleic acid sequence, wherein the gene, an exon, intron, extrachromosomal sequence, or genomic nucleic acid sequence is about 1, 2, 3, 4, 5, 6, 10, 20, 30, 40, 50, 60, 70, 80, 90, 100, 1-100, kilobases in length or is at least about 1, 2, 3, 4, 5, 6, 10, 20, 30, 40, 50, 60, 70, 80, 90, 100, 1-100, kilobases in length. In some embodiments, the use of pairs of RNPs comprising the first and second sgRNA or first and second crRNA molecules may be used to create a polynucleotide molecule comprising the gene, exon, intron, extrachromosomal sequence, or genomic nucleic acid sequence.

In certain embodiments, the target sequence of a sgRNA or crRNA is about 12 to about 25, or about 12, 13, 14, 15, 16, 17, 18, 19, 20, 21, 22, 23, 24, 25, 17-23, or 18-22, nucleotides long. In some embodiments, the target sequence is 20 nucleotides long or about 20 nucleotides long.

In various embodiments, the first and second sgRNA or first and second crRNA molecules are complementary to sequences flanking an extrachromosomal sequence that is within an expression vector.

Aspects of the present subject matter relate to the delivery of multiple components of a gene-editing complex, where the multiple components are not complexed together. In some embodiments, gene editing composition comprises at least one gene editing protein and at least one nucleic acid, wherein the gene editing protein and the nucleic acid are not bound to or complexed with each other.

The present subject matter allows for high gene editing efficiency while maintaining high cell viability. In some embodiments, at least about 10, 20, 30, 40, 50, 60, 70, 80, 90, 95, 99%, 1-99%, or more of the population of cells, or the progeny thereof, become genetically modified after contact with the aqueous solution. In various embodiments, at least about 10, 20, 30, 40, 50, 60, 70, 80, 90, 95, 99%, 1-99%, or more of the population of cells, or the progeny thereof, are viable after contact with the aqueous solution.

In certain embodiments, the gene editing composition induces single-strand or double-strand breaks in DNA within the cells. In some embodiments the gene editing composition further comprises a repair template polynucleotide. In various embodiments, the repair template comprises (a) a first flanking region comprising nucleotides in a sequence complementary to about 40 to about 90 base pairs on one side of the single or double strand break and a second flanking region comprising nucleotides in a sequence complementary to about 40 to about 90 base pairs on the other side of the single or double strand break; or (b) a first flanking region comprising nucleotides in a sequence complementary to at least about 20, 25, 30, 35, 40, 45, 50, 60, 70, 80, or 90 base pairs on one side of the single or double strand break and a second flanking region comprising nucleotides in a sequence complementary to at least about 20, 25, 30, 35, 40, 45, 50, 60, 70, 80, or 90 base pairs on the other side of the single or double strand break. Non-limiting descriptions relating to gene editing (including repair templates) using the CRISPR-Cas system are discussed in Ran et al. (2013) Nat Protoc. 2013 November; 8(11): 2281-2308, the entire content of which is incorporated herein by reference. Embodiments involving repair templates are not limited to those comprising the CRISPR-Cas system.

In various implementations of the present subject matter, the volume of aqueous solution is delivered to the population of cells in the form of a spray. In some embodiments, the volume is between 6.0×10⁻⁷ microliter per cell and 7.4×10⁻⁴ microliter per cell. In certain embodiments, the spray comprises a colloidal or sub-particle comprising a diameter of 10 nm to 100 μm. In various embodiments, the volume is between 2.6×10⁻⁹ microliter per square micrometer of exposed surface area and 1.1×10⁻⁶ microliter per square micrometer of exposed surface area.

In some embodiments, the RNP has a size of approximately 100 Å ×100 Å ×50 Å or 10 nm×10 nm×5 nm. In various embodiments, the size of spray particles is adjusted to accommodate at least about 1, 2, 3, 4, 5, 6, 7, 8, 9, 10, or more RNPs per spray particle.

For example, contacting the population of cells with the volume of aqueous solution may be performed by gas propelling the aqueous solution to form a spray. In certain embodiments, the population of cells is in contact with said aqueous solution for 0.01-10 minutes (e.g., 0.1 10 minutes) prior to adding a second volume of buffer or culture medium to submerse or suspend said population of cells.

In various embodiments, the population of cells includes at least one of primary or immortalized cells. For example, the population of cells may include mesenchymal stem cells, lung cells, neuronal cells, fibroblasts, human umbilical vein (HUVEC) cells, and human embryonic kidney (HEK) cells, primary or immortalized hematopoietic stem cell (HSC), T cells, natural killer (NK) cells, cytokine-induced killer (CIK) cells, human cord blood CD34+ cells, B cells. Non limiting examples of T cells may include CD8+ or CD4+ T cells. In some aspects, the CD8+ subpopulation of the CD3⁺ T cells are used. CD8⁺ T cells may be purified from the PBMC population by positive isolation using anti-CD8 beads. In some aspects primary NK cells are isolated from PBMCs and GFP mRNA may be delivered by platform delivery technology (i.e., 3% expression and 96% viability at 24 hours). In additional aspects, NK cell lines, e.g., NK92 may be used.

Cell types also include cells that have previously been modified for example T cells, NK cells and MSC to enhance their therapeutic efficacy, and use for 3-dimensional cultures, tissue explants, skin grafts, engineered tissues, and the like. For example: T cells or NK cells that express chimeric antigen receptors (CAR T cells, CAR NK cells, respectively); T cells that express modified T cell receptor (TCR); MSC that are modified virally or non-virally to overexpress therapeutic proteins that complement their innate properties (e.g. delivery of Epo using lentiviral vectors or BMP-2 using AAV-6) (reviewed in Park et al, Methods, 2015 August; 84-16.); MSC that are primed with non-peptidic drugs or magnetic nanoparticles for enhanced efficacy and externally regulated targeting respectively (Park et al., 2015); MSC that are functionalised with targeting moieties to augment their homing toward therapeutic sites using enzymatic modification (e.g. Fucosyltransferase), chemical conjugation (eg. modification of SLeX on MSC by using N-hydroxy-succinimide (NHS) chemistry) or non-covalent interactions (eg. engineering the cell surface with palmitated proteins which act as hydrophobic anchors for subsequent conjugation of antibodies) (Park et al., 2015). For example, T cells, e.g., primary T cells or T cell lines, that have been modified to express chimeric antigen receptors (CAR T cells) may further be treated according to the invention with gene editing proteins and or complexes containing guide nucleic acids specific for the CAR encoding sequences for the purpose of editing the gene(s) encoding the CAR, thereby reducing or stopping the expression of the CAR in the modified T cells.

Aspects of the present invention relate to the expression vector-free delivery of gene editing compounds and complexes to cells and tissues, such as delivery of Cas-gRNA ribonucleoproteins for genome editing in primary human T cells, hematopoietic stem cells (HSC), and mesenchymal stromal cells (MSC). In some example, mRNA encoding such proteins are delivered to the cells.

Various aspects of the CRISPR-Cas system are known in the art. Non-limiting aspects of this system are described, e.g., in U.S. Pat. No. 9,023,649, issued May 5, 2015; U.S. Pat. No. 9,074,199, issued Jul. 7, 2015; U.S. Pat. No. 8,697,359, issued Apr. 15, 2014; U.S. Pat. No. 8,932,814, issued Jan. 13, 2015; PCT International Patent Application Publication No. WO 2015/071474, published Aug. 27, 2015; Cho et al., (2013) Nature Biotechnology Vol 31 No 3 pp 230-232 (including supplementary information); and Jinek et al., (2012) Science Vol 337 No 6096 pp 816-821, the entire contents of each of which are incorporated herein by reference.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5,Cmr6,Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2 and in the NCBI database as under accession number Q99ZW2.1. UniProt database accession numbers A0A0G4DEU5 and CDJ55032 provide another example of a Cas9 protein amino acid sequence. Another non-limiting example is a Streptococcus thermophilus Cas9 protein, the amino acid sequence of which may be found in the UniProt database under accession number Q03JI6.1. In some embodiments, the unmodified CRISPR enzyme has DNA cleavage activity, such as Cas9. In certain embodiments the CRISPR enzyme is Cas9, and may be Cas9 from S. pyogenes or S. pneumoniae.In various embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In aspects of the invention, nickases may be used for genome editing via homologous recombination.

In certain embodiments, a Cas9 nickase may be used in combination with guide sequence(s), e.g., two guide sequences, which target respectively sense and antisense strands of the DNA target. This combination allows both strands to be nicked and used to induce NHEJ.

As a further example, two or more catalytic domains of Cas9 (RuvC I, RuvC II, and RuvC III) may be mutated to produce a mutated Cas9 substantially lacking all DNA cleavage activity. A D10A mutation may be combined with one or more of H840A, N854A, or N863A mutations to produce a Cas9 enzyme substantially lacking all DNA cleavage activity. In certain embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. Other mutations may be useful; where the Cas9 or other CRISPR enzyme is from a species other than S. pyogenes, mutations in corresponding amino acids may be made to achieve similar effects.

In certain embodiments, a protein being delivered (such as a Cas protein or a variant thereof) may include a subcellular localization signal. For example, the Cas protein within a RNP may comprise a subcellular localization signal. Depending on context, a fusion protein comprising, e.g., Cas9 and a nuclear localization signal may be referred to as “Cas9” herein without specifying the inclusion of the nuclear localization signal. In some embodiments, the payload (such as an RNP) comprises a fusion-protein that comprises a localization signal. For example, the fusion-protein may contain a nuclear localization signal, a nucleolar localization signal, or a mitochondrial targeting signal. Such signals are known in the art, and non-limiting examples are described in Kalderon et al., (1984) Cell 39 (3 Pt 2): 499-509; Makkerh et al., (1996) Curr Biol. 6 (8): 1025-7; Dingwall et al., (1991) Trends in Biochemical Sciences 16 (12): 478-81; Scott et al., (2011) BMC Bioinformatics 12:317 (7 pages); Omura T (1998) J Biochem. 123(6):1010-6; Rapaport D (2003) EMBO Rep. 4(10):948-52; and Brocard & Hartig (2006) Biochimica et Biophysica Acta (BBA)—Molecular Cell Research 1763(12):1565-1573, the contents of each of which are hereby incorporated herein by reference. In various embodiments, the Cas protein may comprise more than one localization signals, such as 2, 3, 4, 5, or more nuclear localization signals. In some embodiments, the localization signal is at the N-terminal end of the Cas protein and in other embodiments the localization signal is at the C-terminal end of the Cas protein.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis.

Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme corresponding to the most frequently used codon for a particular amino acid.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment 68 algorithm, is about or more than about 50%, 60%, 75%, 80%, 85%, 90%, 95%, 97.5%, 99%, or more. In some embodiments, the degree of complementarity is 100%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq. sourceforge. net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In certain embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions.

CRISPR-Cas technology which facilitates genome engineering in a wide range of cell types is evolving rapidly. It has recently been shown that delivery of the Cas9-gRNA editing tools in the form of ribonucleoproteins (RNPs) yields several benefits compared with delivery of plasmids encoding for Cas9 and gRNAs. Benefits include faster and more efficient editing, fewer off-target effects, and less toxicity. RNPs have been delivered by lipofection and electroporation but limitations that remain with these delivery methods, particularly for certain clinically relevant cell types, include toxicity and low efficiency. Accordingly, there is a need to provide a vector-free e.g., viral vector-free, approach for delivering biologically relevant payloads, e.g., RNPs, across a plasma membrane and into cells. “Cargo” or “payload” are terms used to describe a compound, or composition that is delivered via an aqueous solution across a cell plasma membrane and into the interior of a cell.

The current subject matter relates to delivery technology that facilitates delivery of a broad range of payloads to cells with low toxicity. Genome editing may be achieved by delivering RNPs to cells using some aspects of the current subject matter. Levels decline thereafter until Cas9 is no longer detectable. The delivery technology per se does not deleteriously affect the viability or functionality of Jurkat and primary T cells. The current subject matter enables gene editing via Cas9 RNPs in clinically relevant cell types with minimal toxicity.

The transient and direct delivery of CRISPR/Cas components such as Cas and/or a gRNA has advantages compared to expression vector-mediated delivery. For example, an amount of Cas, gRNA, or RNP can be added with more precise timing and for a limited amount of time compared to the use of an expression vector. Components expressed from a vector may be produced in various quantities and for variable amounts of time, making it difficult to achieve consistent gene editing without off-target edits. Additionally, pre-formed complexes of Cas and gRNAs (RNPs) cannot be delivered with expression vectors.

In one aspect, the present subject matter describes cells attached to a solid support, (e.g., a strip, a polymer, a bead, or a nanoparticle). The support or scaffold may be a porous or non-porous solid support. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present subject matter. The support material may have virtually any possible structural configuration. Thus, the support configuration may be spherical, as in a bead, or cylindrical, as in the inside surface of a test tube, or the external surface of a rod. Alternatively, the surface may be flat such as a sheet, or test strip, etc. Preferred supports include polystyrene beads.

In other aspects, the solid support comprises a polymer, to which cells are chemically bound, immobilized, dispersed, or associated. A polymer support may be a network of polymers, and may be prepared in bead form (e.g., by suspension polymerization). The cells on such a scaffold can be sprayed with payload containing aqueous solution according to the invention to deliver desired compounds to the cytoplasm of the scaffold. Exemplary scaffolds include stents and other implantable medical devices or structures.

### Example 2—Transfection of iPSCs

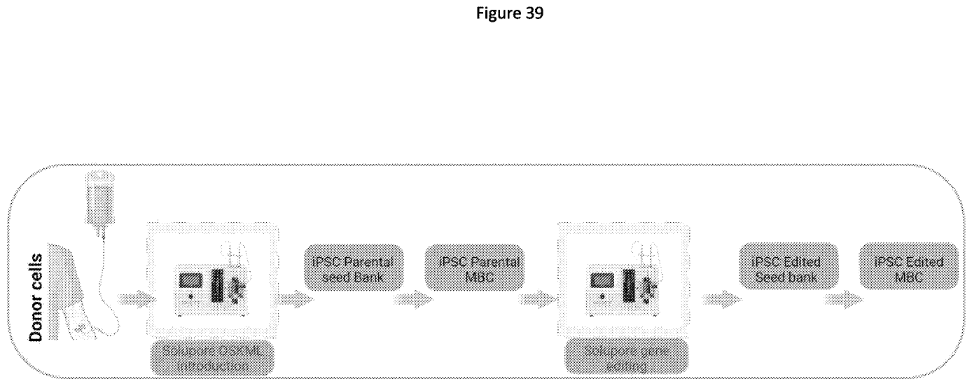

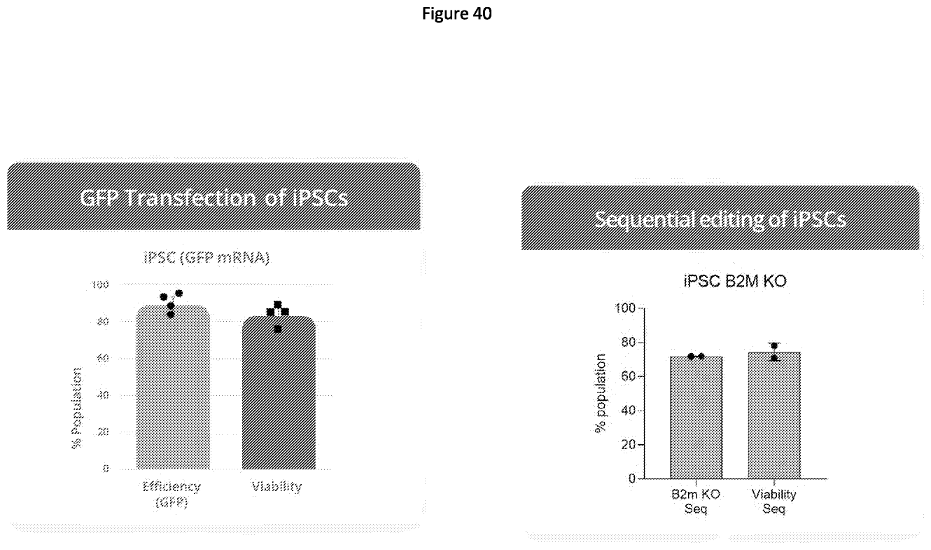

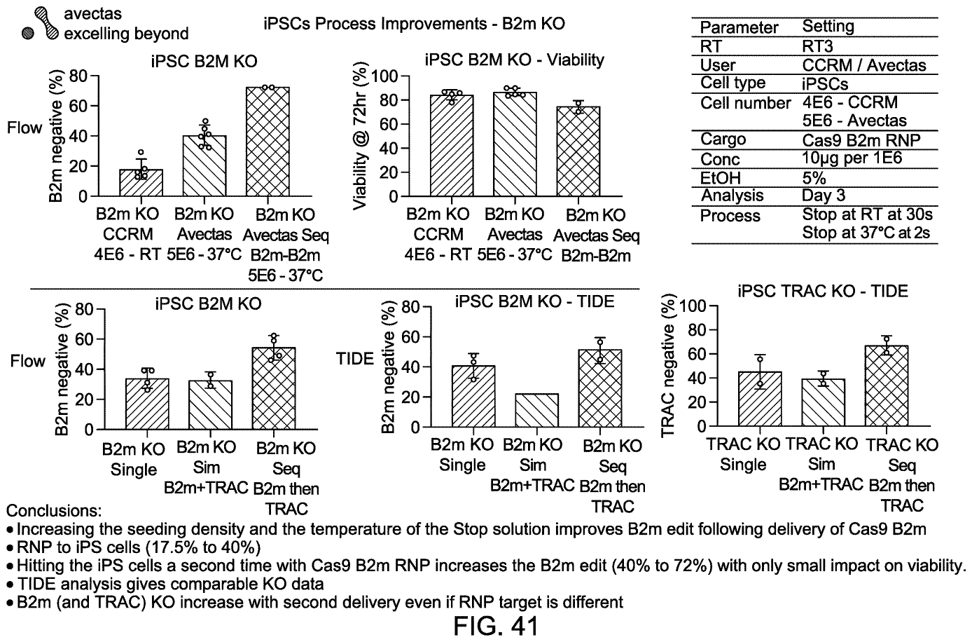

Example 2 includes transfection of iPSC using a stop solution having a temperature of 37 °C. The delivery system demonstrated high edit efficiency and viability for GFP mRNA and RNPs. FIG. 39 and FIG. 40. Transfection of the iPSCs includes opportunities for cell reprogramming and gene editing. The increase in temperature of the stop solution from ambient temperature (20-25 °C) to 37 °C increased the transfection efficiency of the process. In other examples, the process further includes decreasing the process time for the addition of stop solution (e.g., post spray) from 30 seconds to 2 seconds. The decrease in process time for the addition of stop solution increased the transfection efficiency of the process. FIG. 41.

The methods described herein further include increasing the starting iPSC cell density for transfection, from 4×10⁶ cells to 5×10⁶ cells, and the increase in cell (iPSC cell) density increased the transfection efficiency and maintained iPSC culture health post transfection.

In other examples, a second transfection of iPSCs was performed using the delivery system and method. The second transfection was performed between half hour to one hour post the initial transfection increased the transfection efficiency of the process. Moreover, additional transfections (e.g., more than 2) of iPSCs are performed using the delivery system. Additional transfections can include 2, 3, 4, 5 or more transfections.

### Example 3: Effect of Increased Process Temperature Using iPSCs

The increase in process temperatures as described herein using iPSCs increased the overall CRISPER Cas9 RNP β2M knock-out efficiencies and maintained high cell viability. The increase in process temperature and starting number of iPSCs also increased transfection efficiencies and viability of the cells post-transfection. FIG. 41.

### Example 4: Effect of Alcohol on RNP-Edit Efficiency

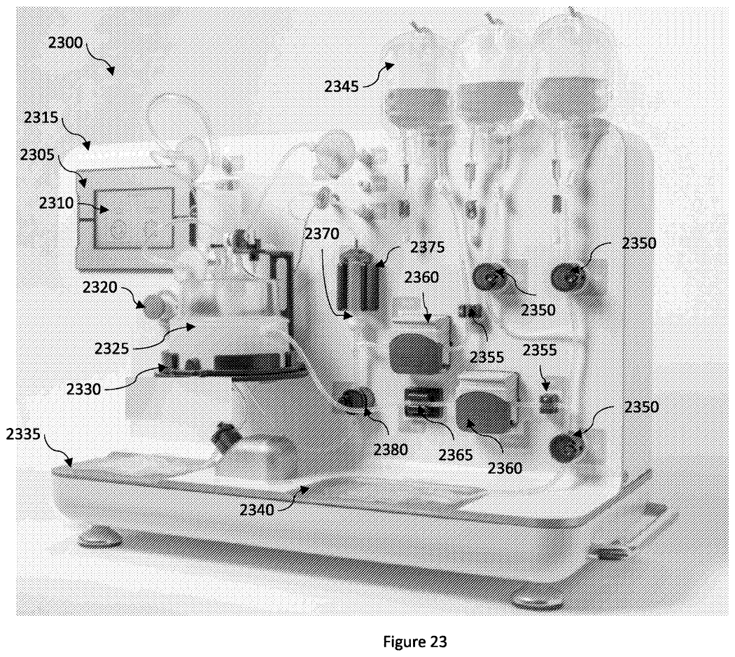

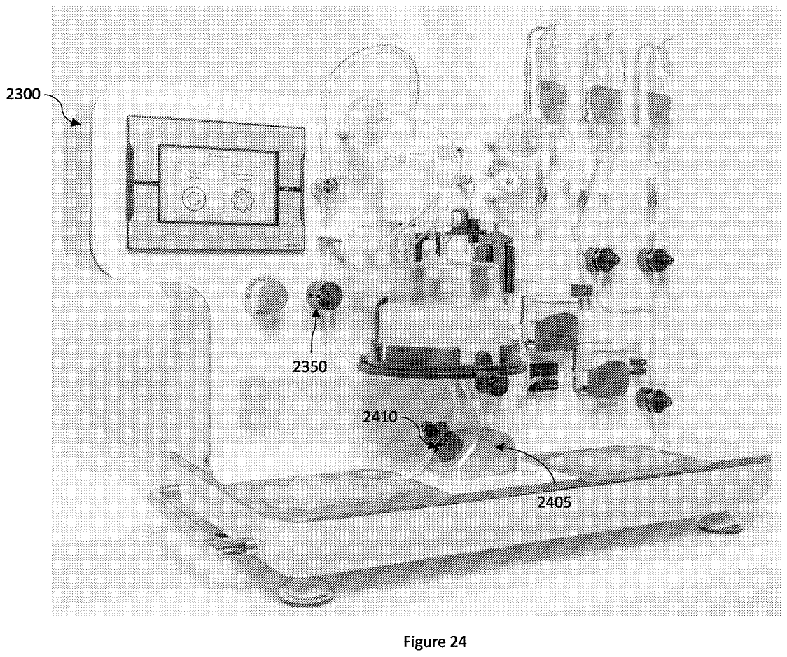

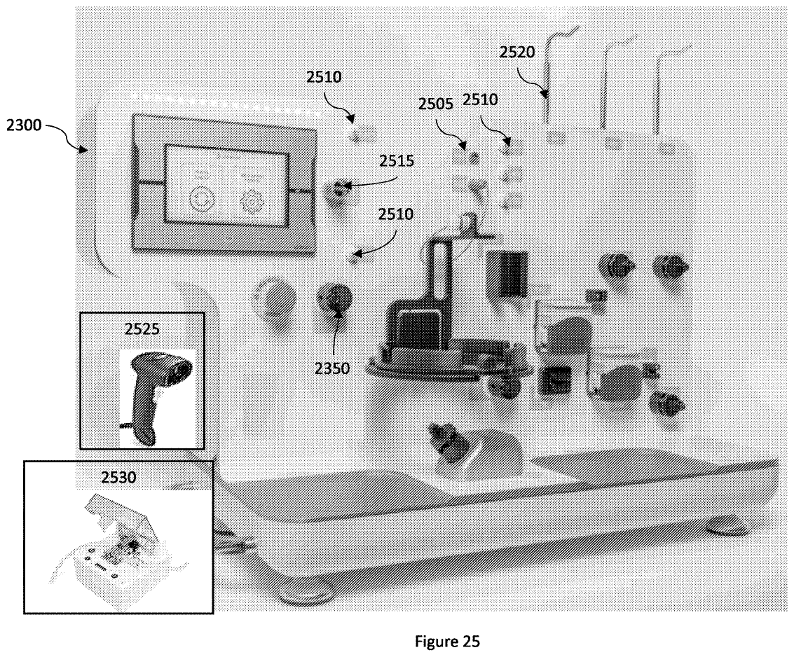

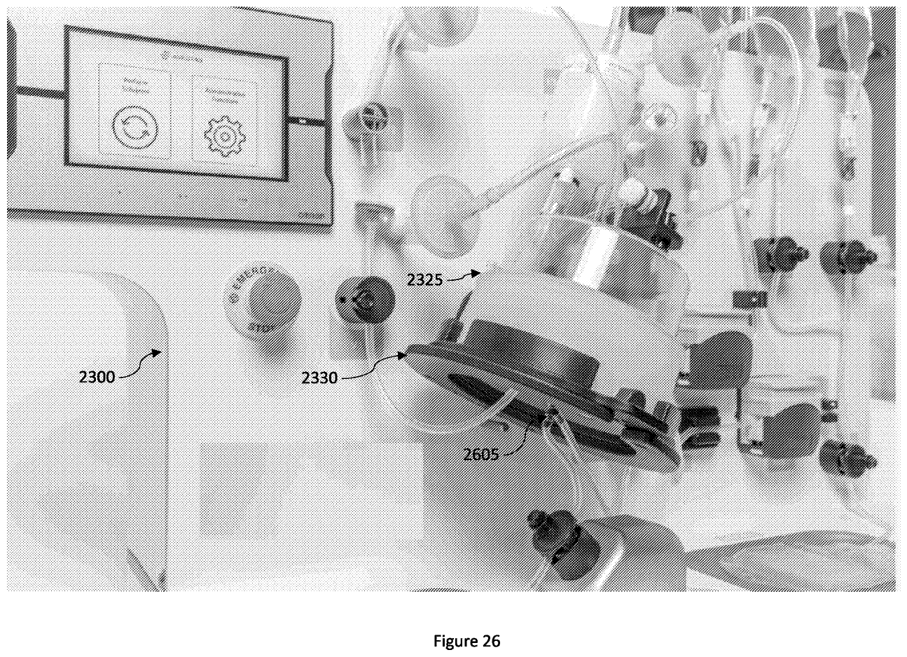

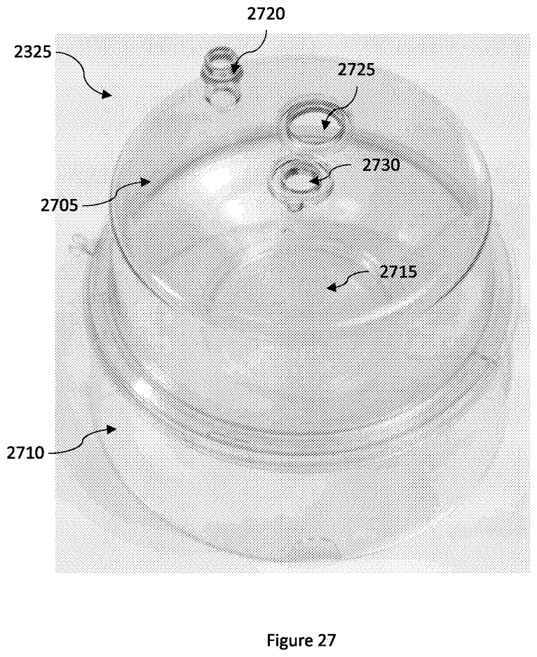

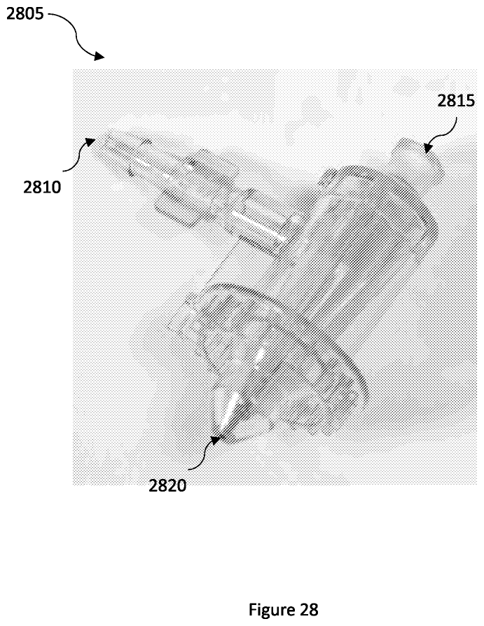

Effect of alcohol on RNP (ribonucleoprotein)-edit efficiency post-delivery by the example delivery platforms illustrated in FIGS. 1 and 23.

Experiments were performed to determine the effect alcohol (e.g., ethanol) had on RNP-edit efficiency post-delivery using the example delivery platforms illustrated in FIGS. 1 and 23. Additionally, the experiments were performed to ascertain an optimal ethanol concentration for editing following delivery of RNP by the example delivery platforms illustrated in FIGS. 1 and 23. For example, the maximum ethanol concentration which allowed for optimal Cas9-induced edit was determined. An increase in ethanol allowed for more cargo delivery to the cell, and thereby allowing for greater edit efficiency.

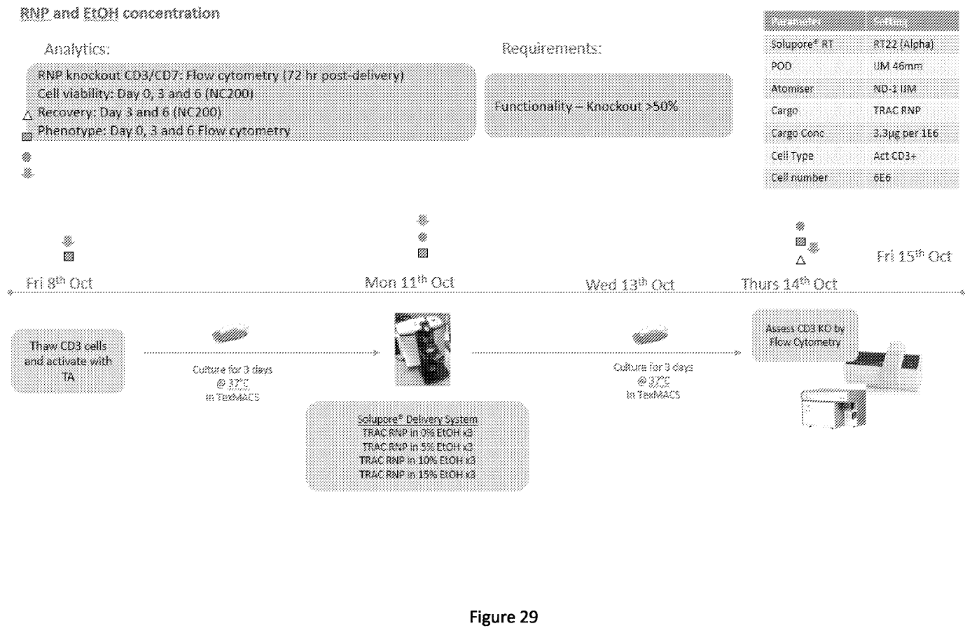

Cas9 RNP-TRAC (T cell receptor alpha constant) sgRNA (single guide RNA) was prepared at 2:1 ratio at 0.4 μg/μL (equiv to 3.3 μg per 1×10⁶ cells); S Buffer (32.5 mM sucrose; 106 mM potassium chloride; 5 mM HEPES) solutions were prepared with 0, 5, 10 and 15% ethanol with RNP and the experiments were carried out on the example delivery platforms illustrated in FIGS. 1 and 23. with the S buffer solutions at each ethanol concentration. The TRAC guide RNA sequence: AGAGTCTCTCAGCTGGTACA (SEQ ID NO: 1). In embodiments, at least two exogenous cargos are simultaneously delivered, meaning the two exogenous cargos are delivered at the same time (e.g., dual delivery). For example the immune cell (comprising an exogenous cargo), may be manipulated to comprise a second exogenous cargo. The experimental design is shown in FIG. 29.

“S Buffer” includes a hypotonic physiological buffered solution (78 mM sucrose, 30 mM KCl, 30 mM potassium acetate, 12 mM HEPES) for 5 min at 4 °C (Medepalli K. et al., Nanotechnology 2013; 24(20); incorporated herein by reference in its entirety). In some examples, potassium acetate is replaced with ammonium acetate in the S Buffer. S buffer is further described in international application WO 2016/065341, e.g., at ¶ [0228]-[0229] and incorporated herein by reference in its entirety. For example, the S buffer used in series of experiments described herein included 32.5 mM sucrose; 106 mM potassium chloride; and 5 mM HEPES.

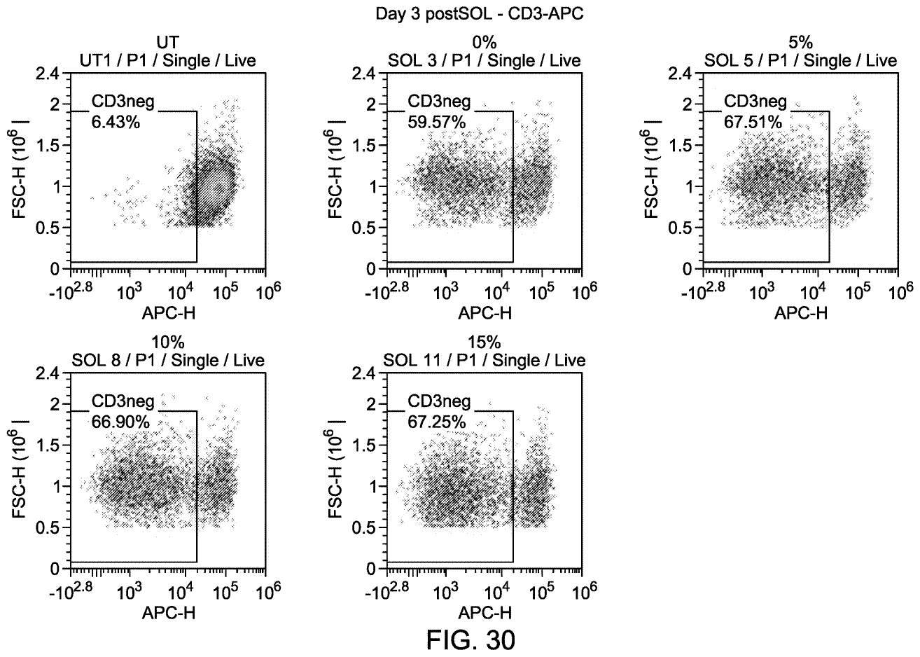

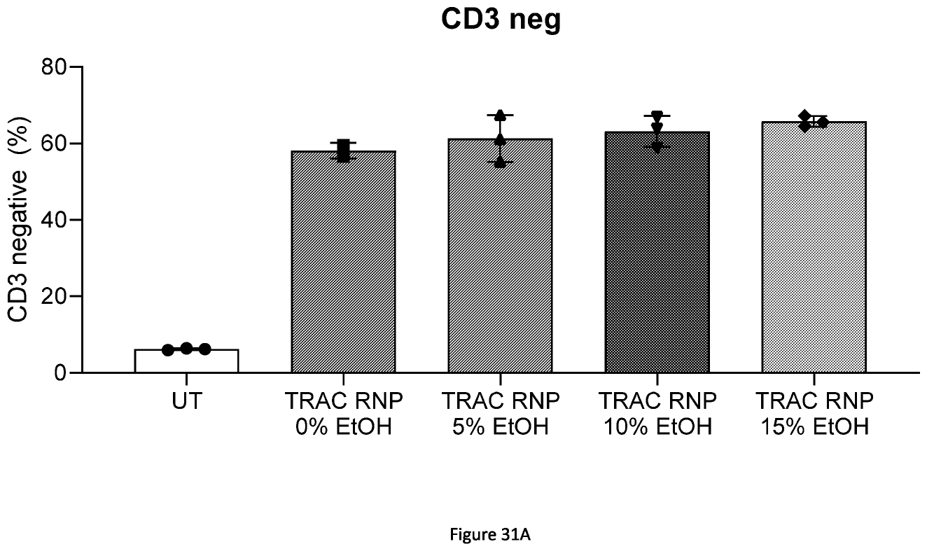

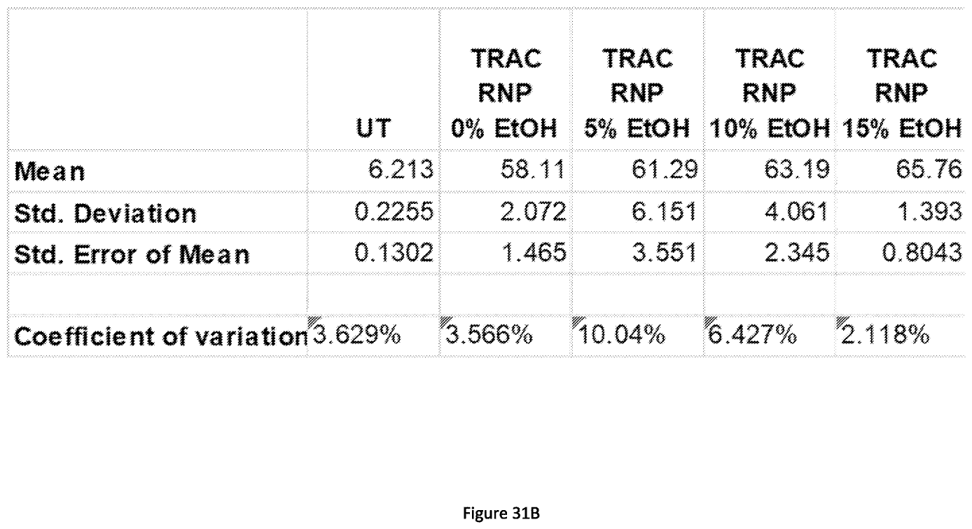

Conclusion: CD3 (cluster of differentiation 3) edit efficiencies (e.g., monitoring TRAC RNP) at each ethanol concentration was tested post-delivery using the example delivery platforms illustrated in FIGS. 1 and 23. See FIG. 30 depicting representative flow cytometry plots from cells stained with an antibody targeting CD3 (gated off the live population) and FIG. 31.

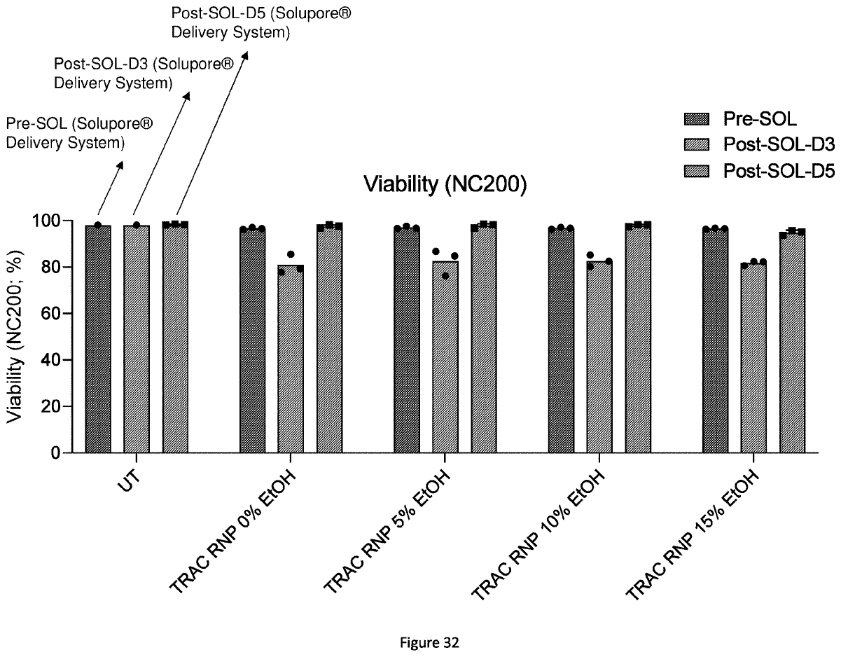

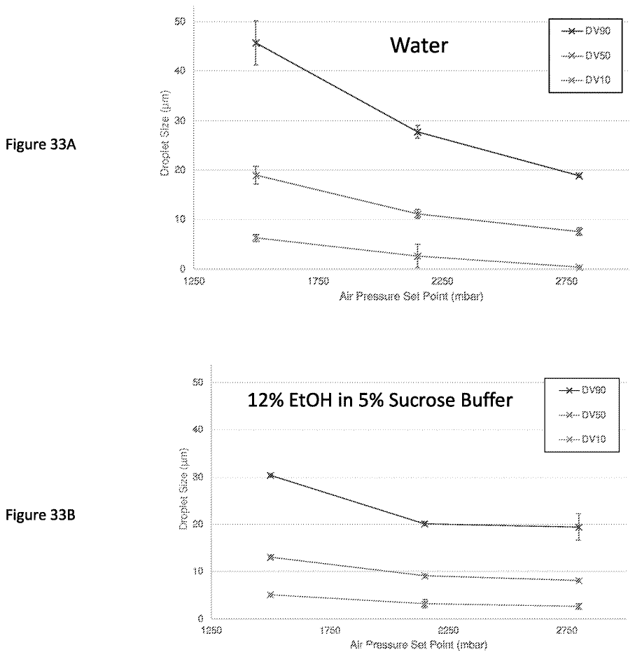

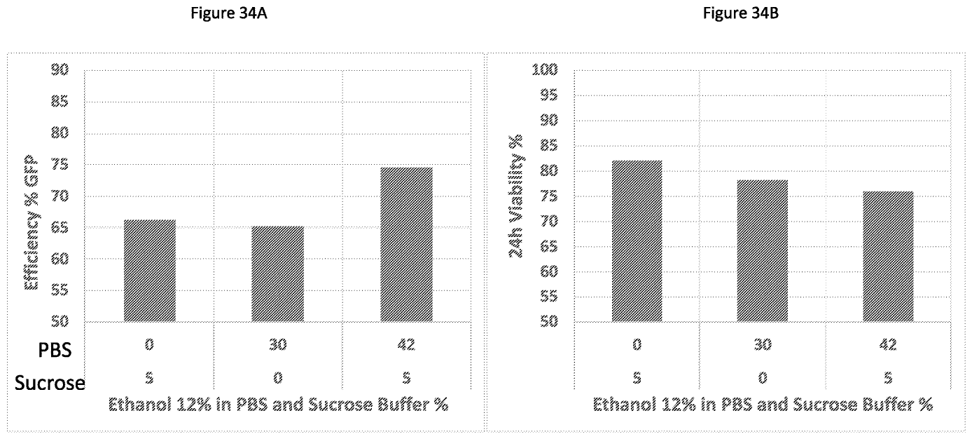

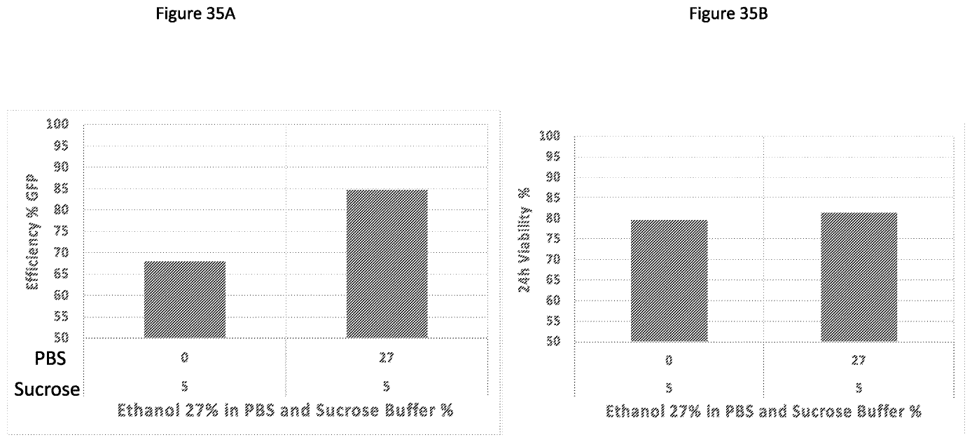

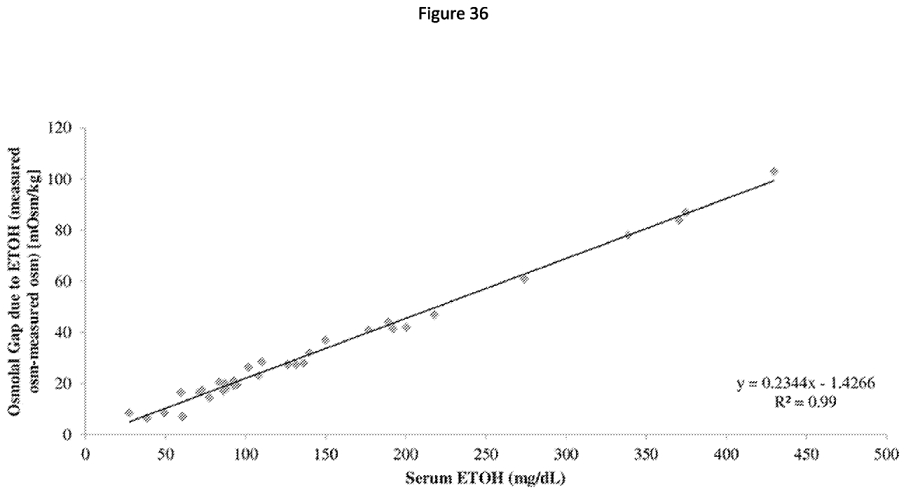

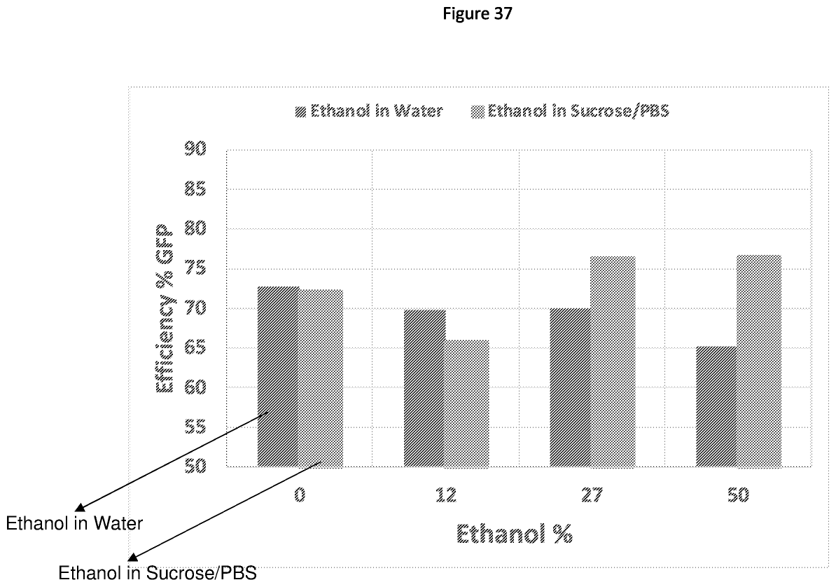

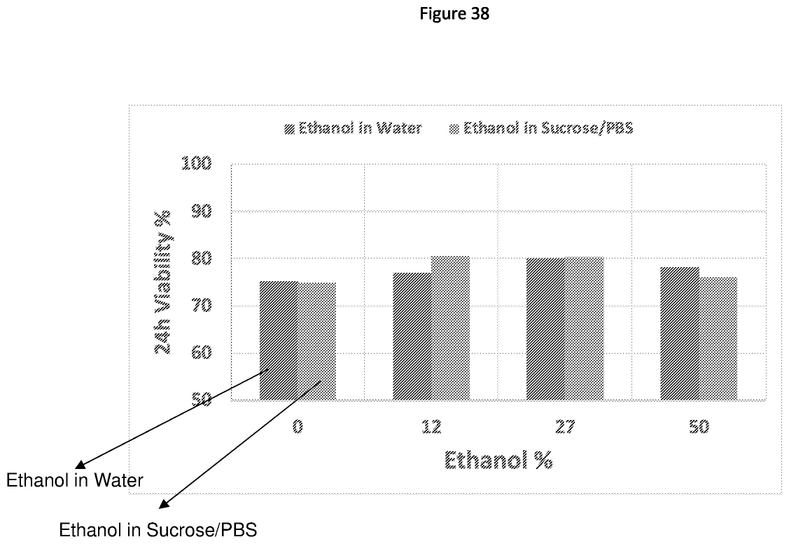

FIG. 31A shows a bar graph showing that the level of CD3 edit increased modestly with increasing concentrations of ethanol (0% EtOH and 58% CD3 edit to 15% EtOH and 66% CD3 edit), and the results are further summarized in the table in FIG. 31B. The percent viability at the increasing ethanol concentrations, and time points consisting of pre-delivery, post-delivery (day 3) and post-delivery (day 5) are summarized in the bar graph in FIG. 32.

### Example 5: Study of Droplet Size Using the Delivery Platform

FIGS. 33A and 33B illustrate droplet size versus pressure of atomization for the example delivery platform when employing a 0% EtOH delivery solution (FIG. 33A, no alcohol) and with 12.5% alcohol (FIG. 33B). In FIGS. 33A and 33B, DV90 indicates that the portion of particles with diameters smaller than this value is 90%, DV50 indicates that the portion of particles with diameters smaller than this value is 50%, and DV10 indicates that the portion of particles with diameters smaller than this value is 10%.

In some implementations, aqueous solutions without ethanol showed a larger droplet size (for a given pressure for atomizing the solution), which required additional consideration of process conditions to give optimal spay coverage of cells with cargo for transfection.

In some implementations, when the platform is utilizing a 0% ethanol delivery solution, additional wash steps can be omitted. The on/off switching speed of the spray delivery can remain constant. Similarly, the plume and nozzle design can used for ethanol or no ethanol solutions. As described in more detail in Example 6, the system can also provide for delivery using a hypertonic solution (e.g., a much higher salt concentration in the delivery solution).

For cells of approximately 10 μm in diameter (e.g. human T cells) FIGS. 33A and 33B are line graphs showing that the spray droplet size required higher atomisation pressures to be applied to maintain the droplet size range closer to cell size, including to avoid excessively large droplets. Droplet size was measured as D90 using Malvern Mastersizer 3000 laser diffraction apparatus (available from Malvern Panalytical Ltd., Malvern, United Kingdom; see, (<https://www.malvernpanalytical.com/en/products/product-range/mastersizer-range/mastersizer-3000>).

In some implementations, the example delivery platform can utilize a pressure where a distribution of spray droplet (e.g., particle) size distribution can include a size range where D90 is not more than 5 times cell size, a range where D90 is not more than 3.3× cell size, and/or a range where D90 is not larger than about 2× cell size. In examples, the cells are iPSCs that have a diameter of about 15-30 μm. In some embodiments, the cells have a diameter of about 15 μm, or about 20 μm, or about 25 μm, or about 30 μm. The spray droplet size (when using iPSCs) requires higher atomization pressures to be applied to maintain the droplet size range closer to cell size.

### Example 6: Delivery of Functional Cargo to Primary T Cells and Editing of iPSCs Using CRISPR

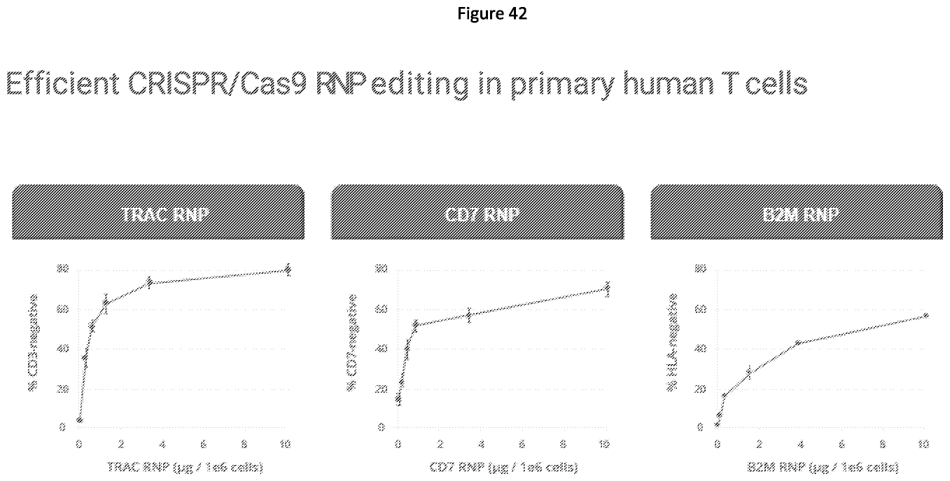

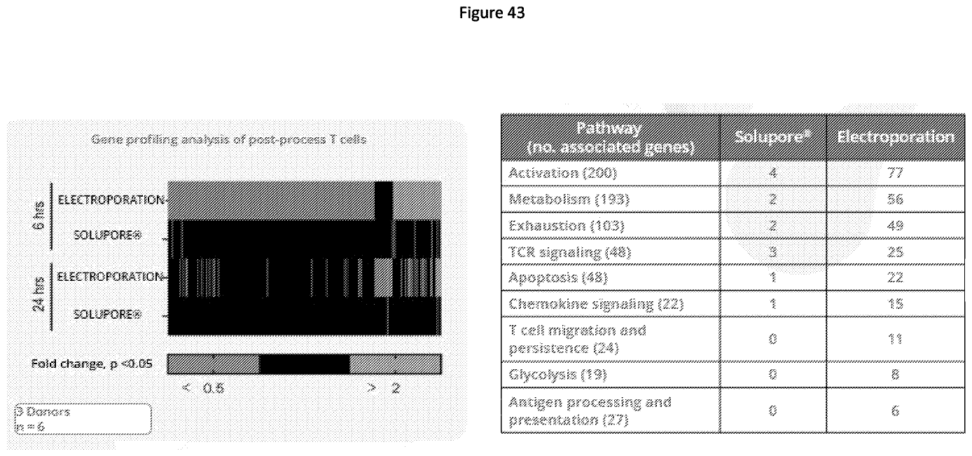

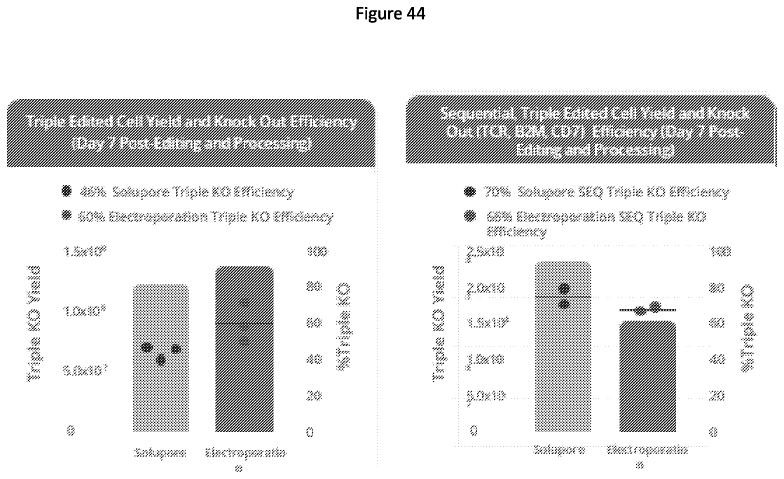

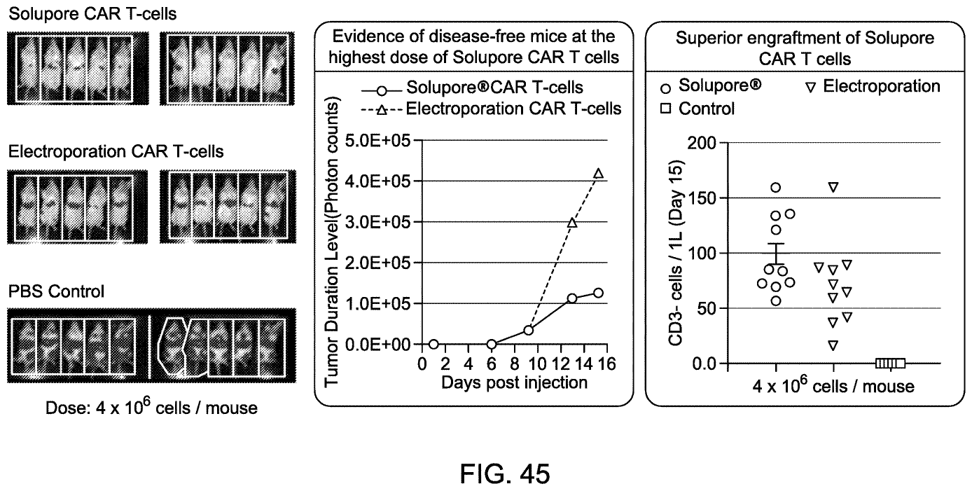

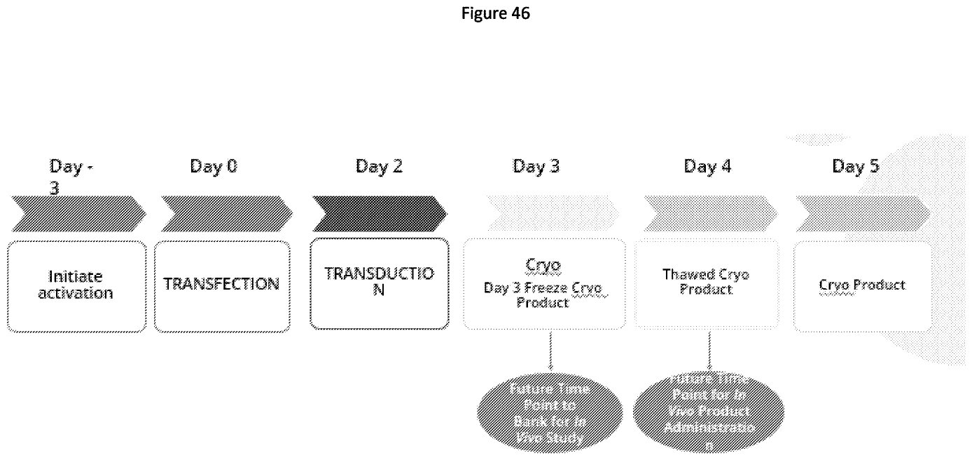

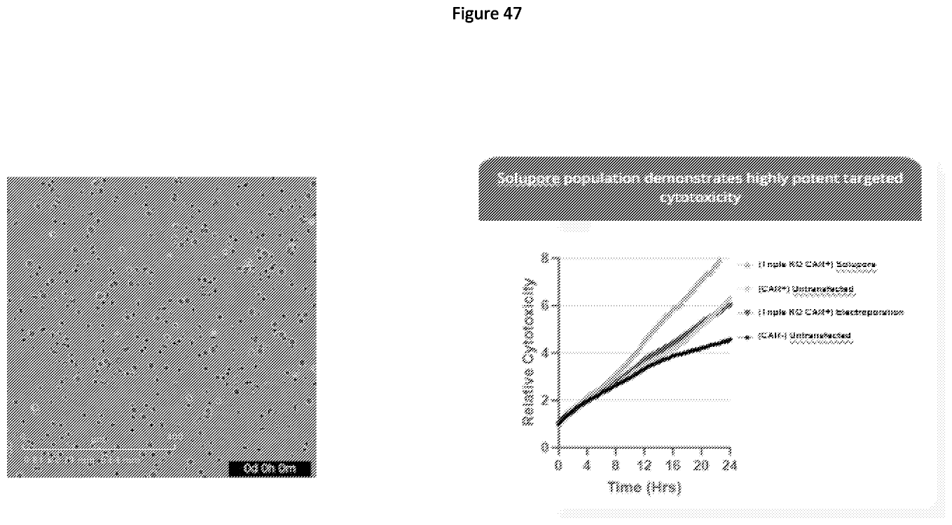

In implementations, the methods delivered functional cargo to primary T cells, where efficient CRISPR/Cas9 RNP editing in primary human T cells was observed (FIG. 42). The methods demonstrated minimal perturbation of T cells (FIG. 43). The methods further demonstrated the delivery of multiplex and sequential complex edits. The method demonstrated higher edited cell yield, with superior cell function as compared to electroporation (FIG. 44). In implementations, the method demonstrated enhanced in vivo cell functionality (there was evidence of disease-free mice with superior engraftment of the described CAR T cells (FIG. 45). In implementations, triple knockout CAR T cells demonstrated potential targeted cell cytotoxicity when co-cultured with CD19+ Raji cells (FIG. 47). Cells processed as described herein, had less apoptotic cells in comparison to electroporation, and thereby showing superior cell health post process, wherein 25% of the remaining cells from electroporation went through apoptosis and only 12.5% of the cells were apoptotic using the instant methods. This was measured using a dye that tracked caspase-3 activity to monitor apoptosis in the early and late stages. In implementations, the method demonstrated a higher % of stem cell memory T cells retained post process in comparison to electroporation. This was measured 4 days post transfection, which is 2 days post transduction.

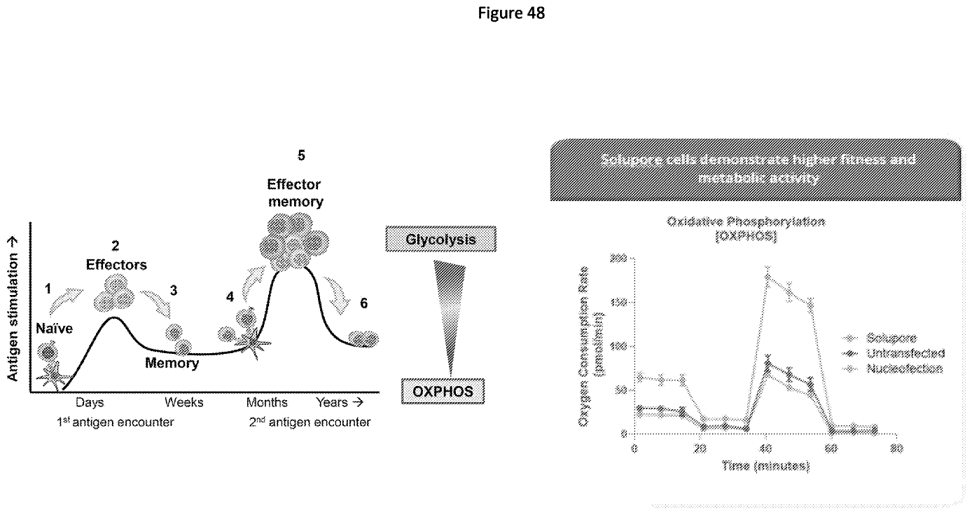

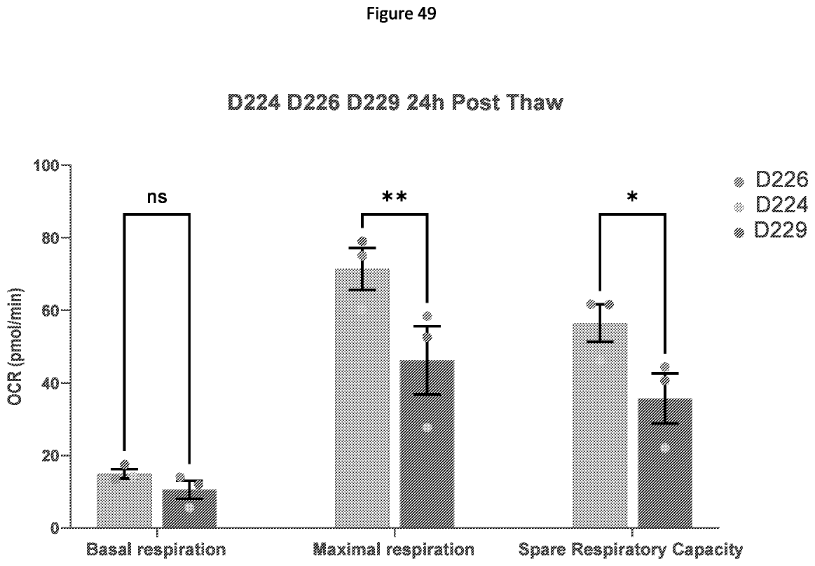

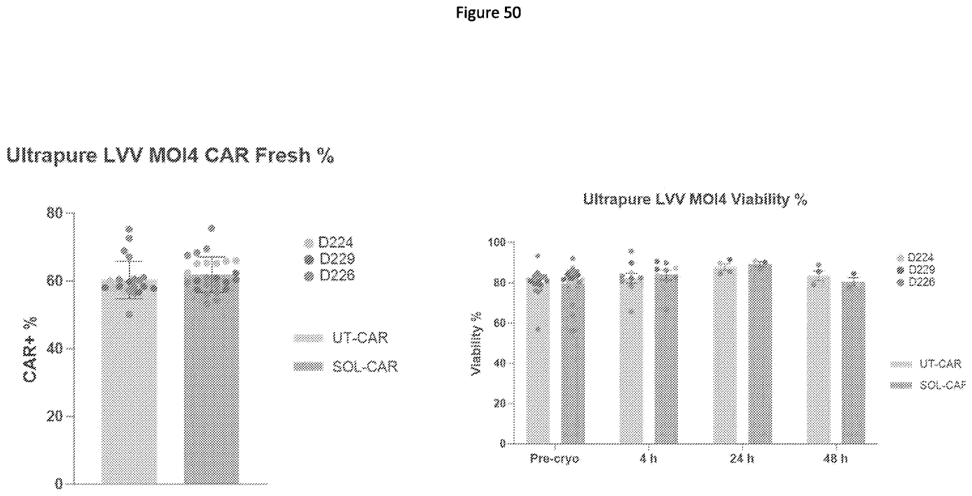

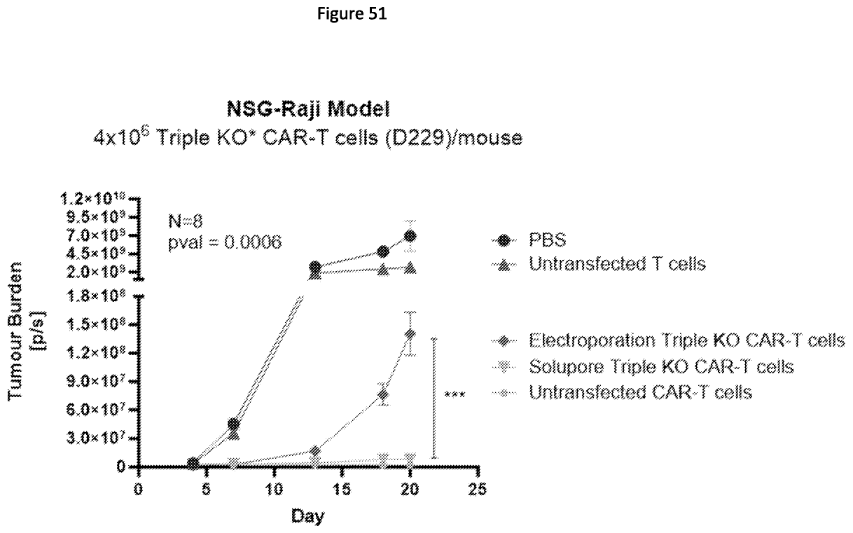

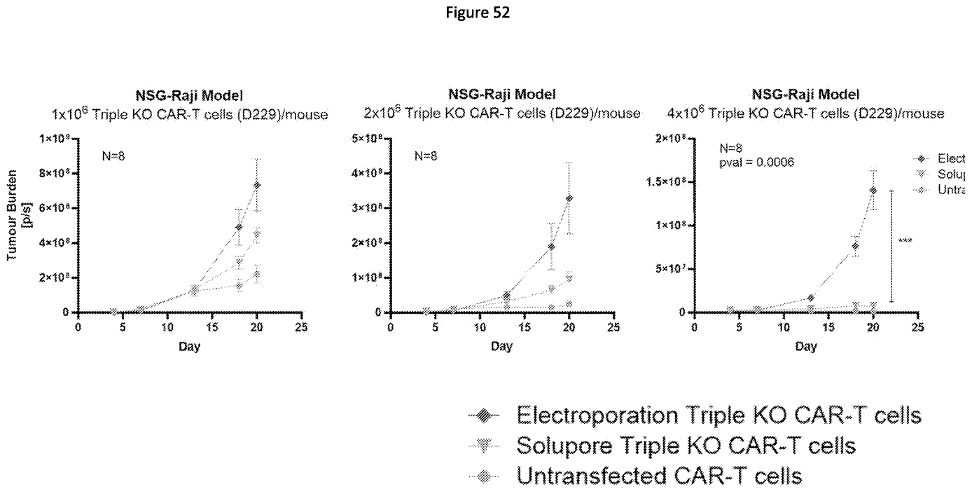

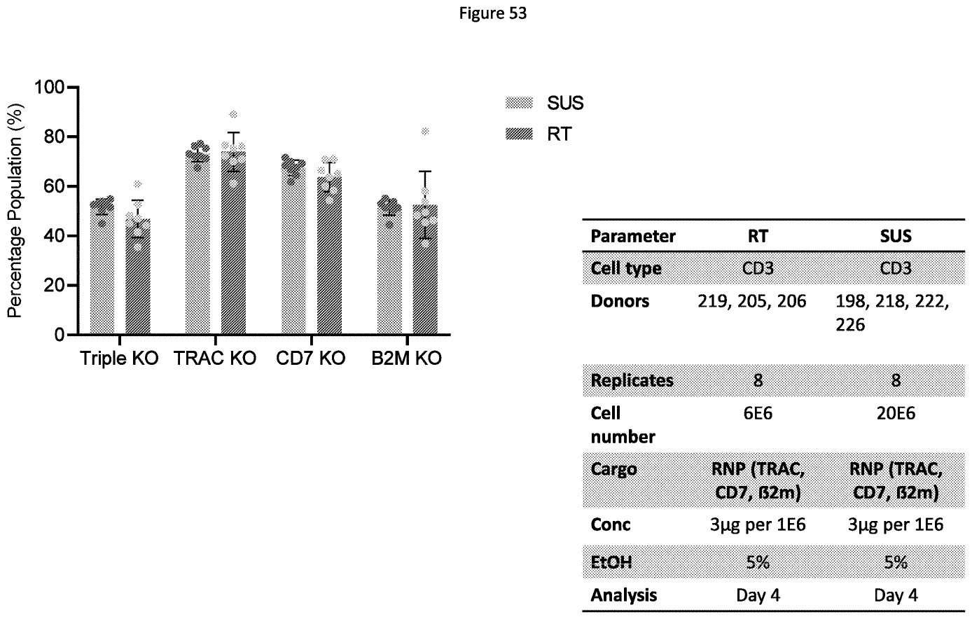

In implementations, the methods demonstrated superior cell health and phenotype. Car T cells engineered using the described methods were less apoptotic than electroporation, retained a younger memory phenotype, and were more metabolically active, which a higher maximum respiratory rate (oxygen consumption rate) (FIG. 48, FIG. 49, FIG. 50, FIG. 51).

In implementations, the methods herein demonstrated a dose-dependent protection against tumor growth (FIG. 52). As w the dose of CAR-T cells was increased in each cohort of mice, the instant methods demonstrated a dose dependent tumour growth inhibition similar to the untransfected control group, as compared to electroporation/standard technology which fails to control tumour growth in all three respective dosing groups, the tumour burden between Solupore and electroporation in the higher dose is statistically significant (p val=0.0006), whereas the difference between Sol and UT is not significant (pval=0.06).

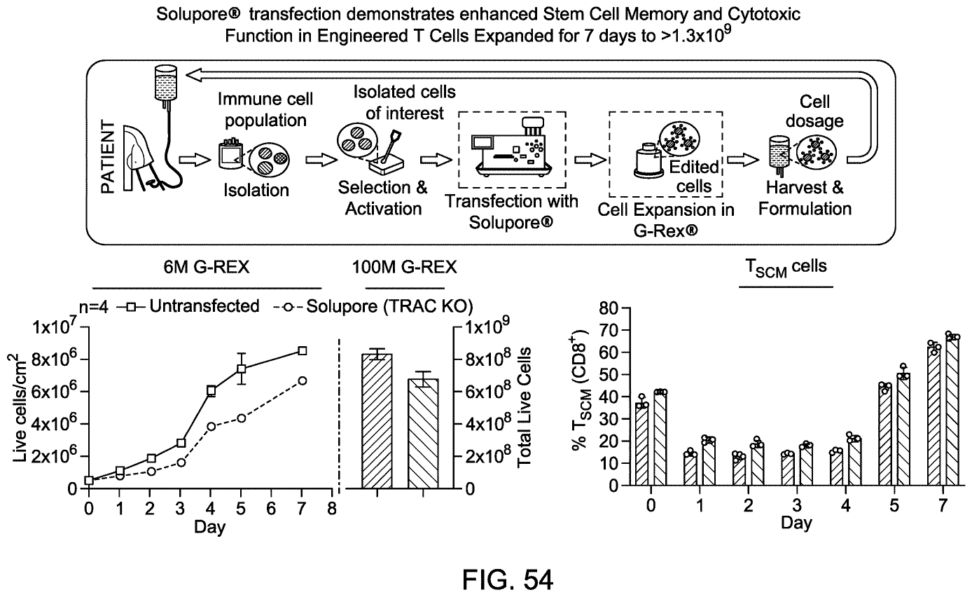

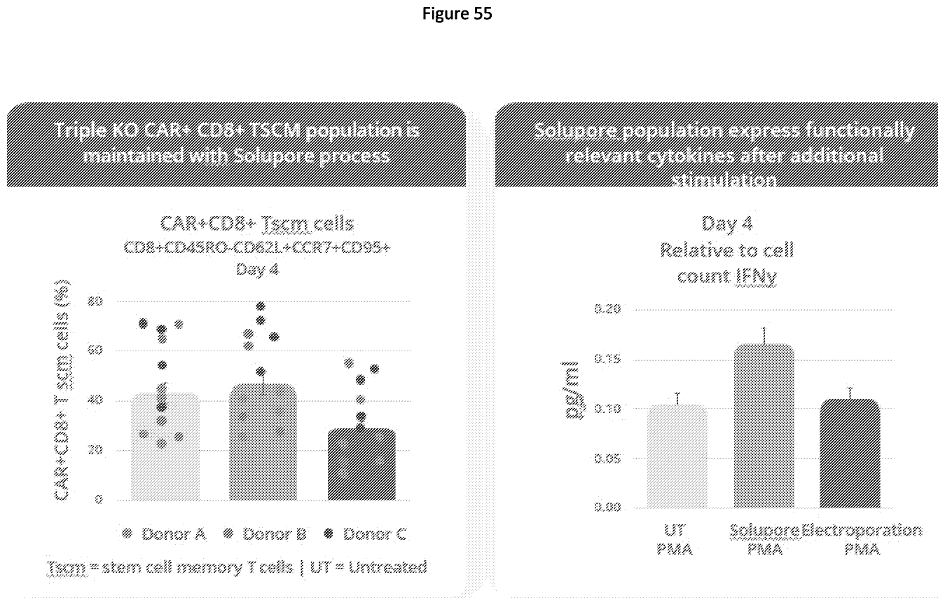

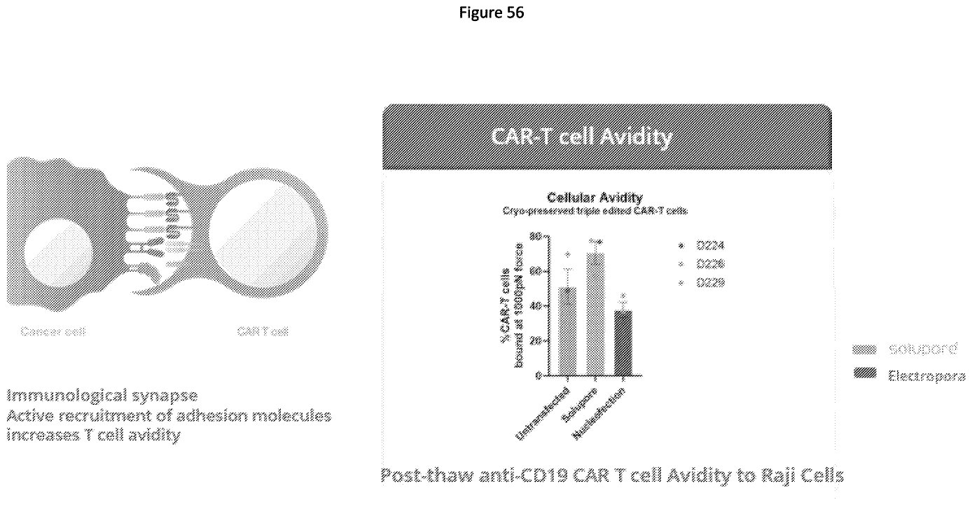

In implementations, the transfection methods demonstrated enhanced stem cell memory and cytotoxic function in engineered T cells expanded for 7 days (FIG. 54). In implementations, the methods demonstrated superior cell health and phenotype (FIG. 55). In implementations, the methods demonstrated a superior ability to bind target cells after cryopreservation (FIG. 56). Another readout to predict modified cells ability to kill target cells in vivo by focusing in on the synapse and the strength of that synapse so looking at cell health as whole. The antitumor response is driven by the overall collection of interactions that take place between the T cell and the tumor cell. T cell binding avidity drives the tumor killing through the formation of a strong and mature immunological synapse. In this specific synapse state, the interaction forces substantially increase because of the active recruitment of many adhesion molecules at the cell-cell interface. Only in this state the T cells can execute the killing by very locally delivering cytotoxic molecules. As described herein, cells were transfected have been edited by CRISPR RNP targeting 3different targets. The cells were CAR transduced at 2 days post-transfection and then transfected/transduced cells were observed for that synapse binding ability. There is a significant difference between the described methods and electroporated cells. The 10% difference can translate to a significant difference in how the cells perform in vivo. Cells that were processed using the described methods were functionally healthy. The described methods demonstrated that cells post-process had a significantly higher binding ability in comparison to electroporated cells. This has a direct correlation to the efficacy of cells performance in vivo and compliments all other analytical readouts demonstrating the superior cell health and function of cells processed.

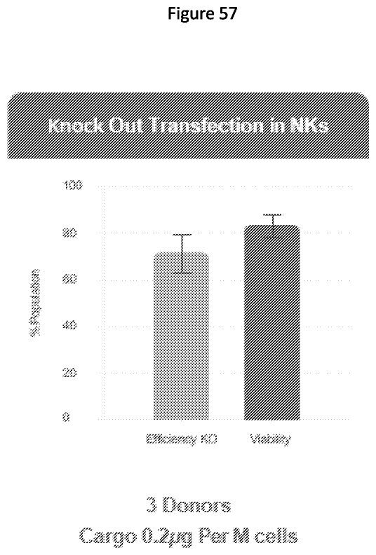

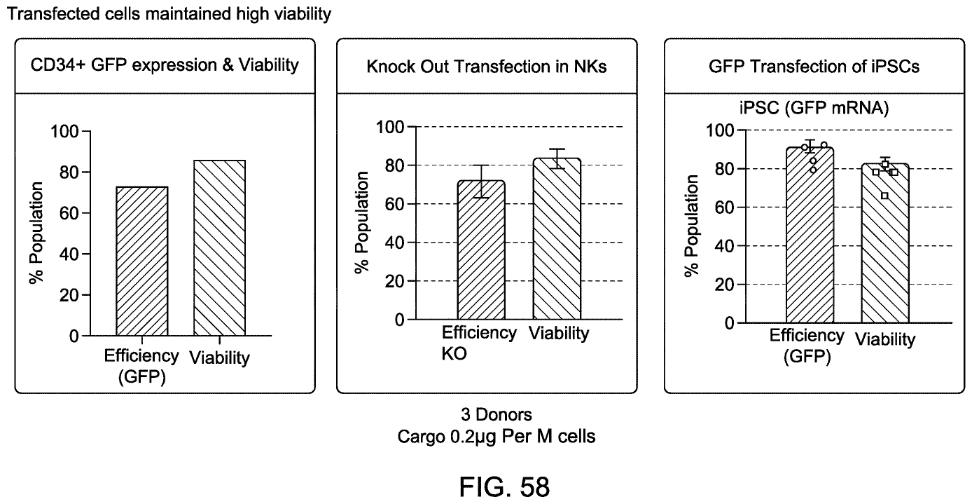

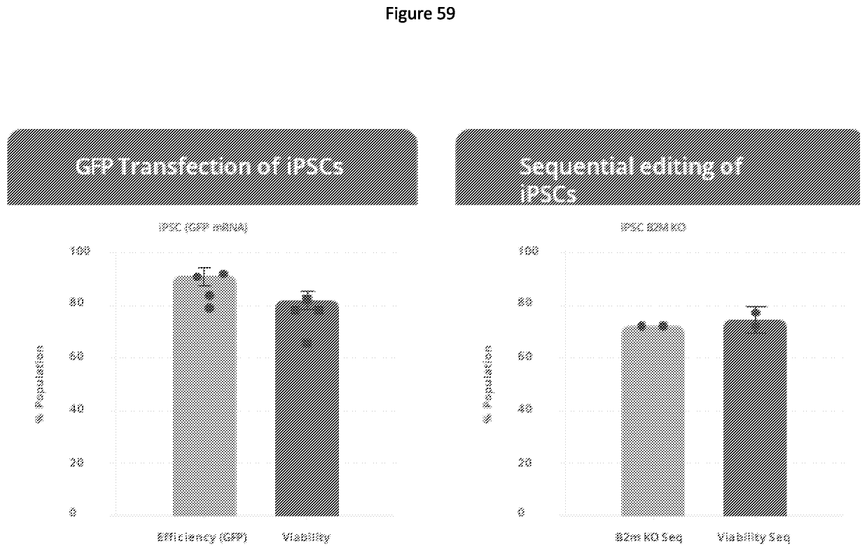

In implementations, the methods provided for efficiently transfecting a variety of cell types, including NK cells (FIG. 57, FIG. 58). Transfection of both iPSC and NK was determined with a variety of cargo leading to excellent efficiency and viability post process. iPSC model cargo GFP mRNA and post-process we have a healthy cell culture post process with a good expansion. NK-TIGIT KO. In implementations, the methods efficiently transfected iPSC (FIG. 59). Transfection of iPSCS provided new options for cell reporgramming and gene editing.

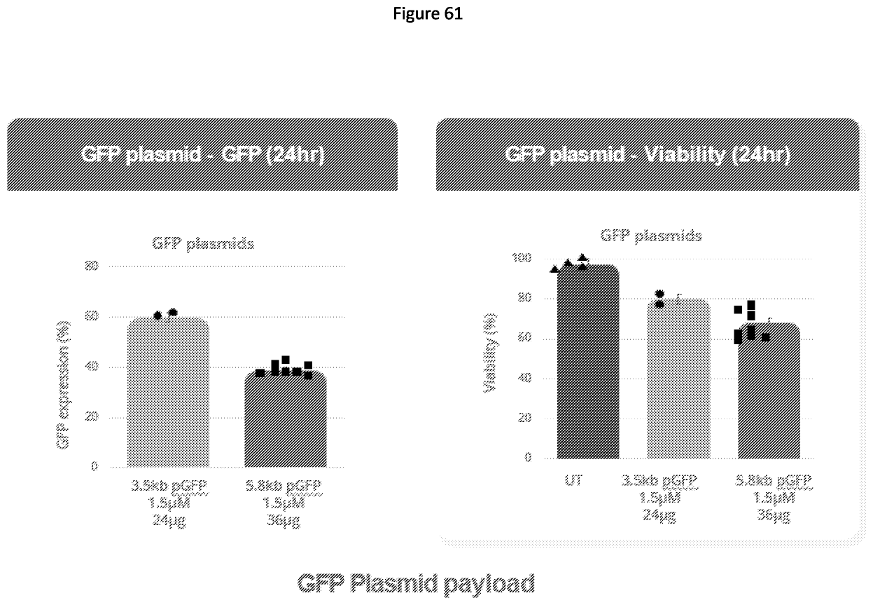

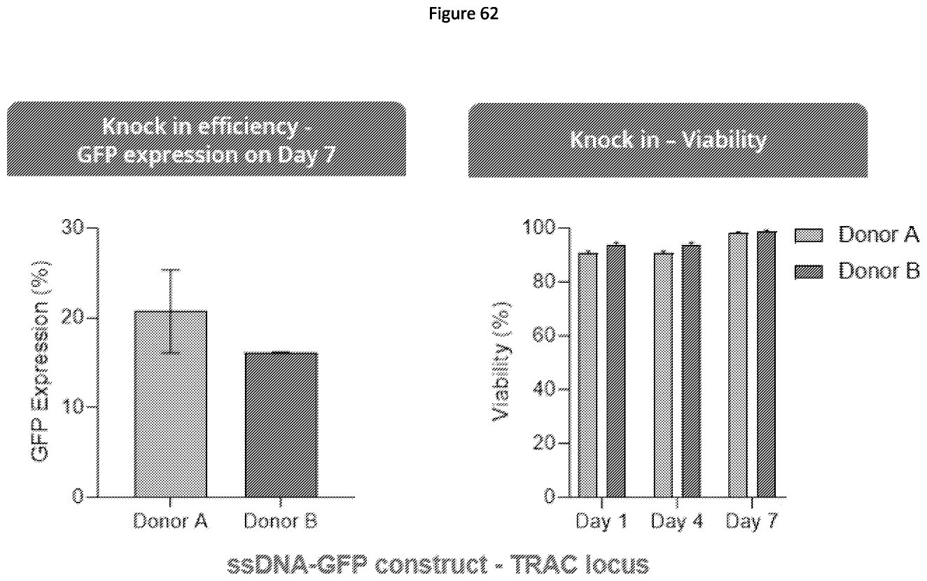

In implementations, the methods demonstrated delivery of large plasmid payloads while maintaining cell viability (FIG. 61). In implementations, the methods demonstrated good knock-in efficiency with excellent viability across two donors (FIG. 62). The cells were activated by Transact for 2 days prior to transfection with IL-7 and IL-15. Cells were pre-treated for 30 min prior to transfection with NATE (innate immune response inhibitor) 6E6 transfected on Day 0. Cargo: TRAC RNP—6 ug per 1E6; 4:1 sgRNA to Cas9 OR ssDNA CTS eGFP from Genscript—4 ug per 1E6 OR RNP+ssDNA complexed prior to delivery (theory-NLS on Cas9 transports ssDNA into nucleus). Cells were seeded into 24-well G-Rex plate and spiked with M3814 (HDR Enhancer; 1 μM) and dNTPs (50 μM) for 24 hr Cell counts carried out on Day 1, 4 and 7 GFP expression assessed on Day 4 and 7.

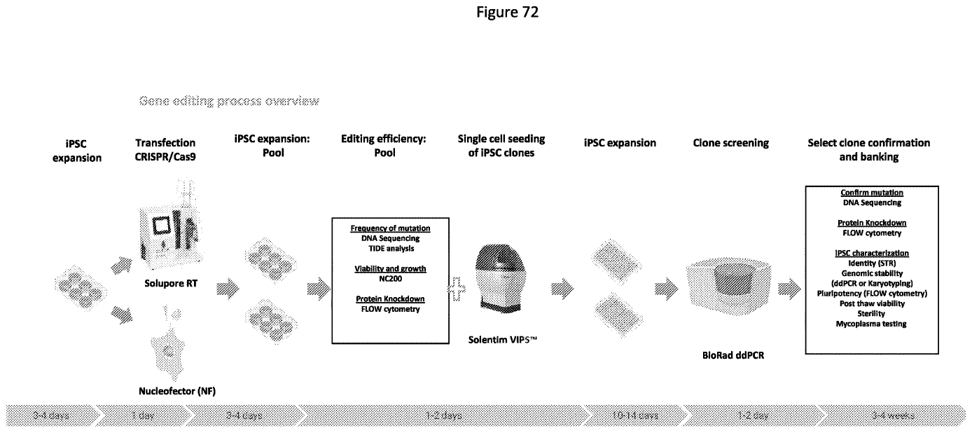

Induced pluripotent stem cells (iPSCs) hold therapeutic potential due to their ability to differentiate into various cell types; this application is further enhanced by gene editing. Existing delivery methods for gene editing tools encounter challenges, such as excess cargo load and prolonged timelines for multi-step editing. The described methods and devices were employed to generate single and multi-gene edited iPSCs. The multiplex capabilities combined with the ability to deliver sequentially, enabled diverse cargo delivery options, including knockout (KO) and knock-in (KI) editing, while maintaining cell health and reducing cell derivation workflow time (FIG. 72).

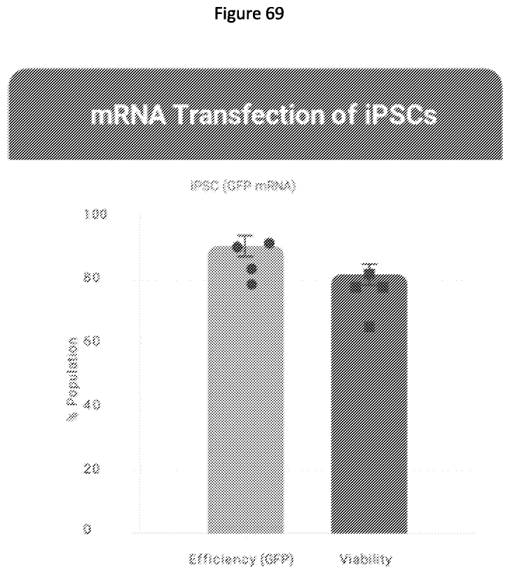

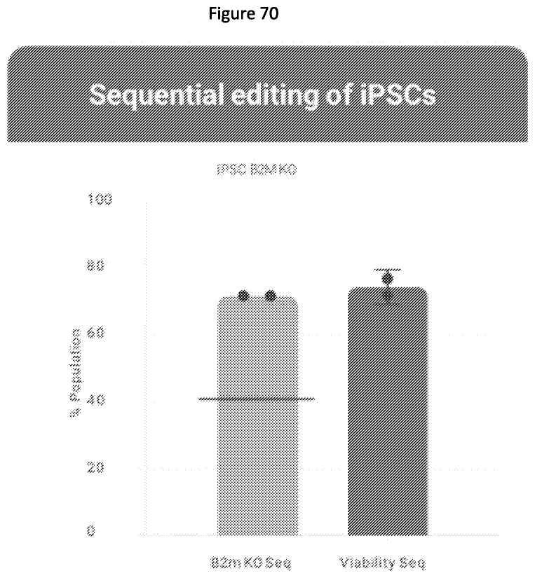

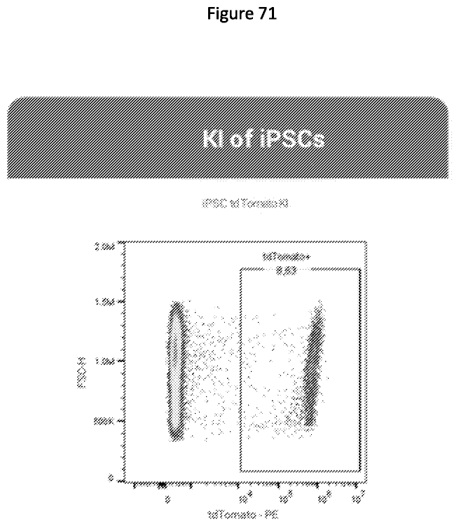

In implementations, the methods described herein sequentially transfected iPSCs. iPSCs were transfected sequentially with mRNA and showed high viability (FIG. 69, FIG. 70, and FIG. 71). 5 million iPS cells (PS11) were transfected using Solupore RT. A single GFP mRNA transfection yielded ~90% transfection efficiency at 24 hr. Cas9 RNP targeting B2m was delivered to iPS cells on 2 separate occasion, with 1 hr apart, to yield ~70% knock of B2m on day 4 post-transfection. Cas9 RNP targeting the safe harbour site AAVS1 in combination with a ssDNA HDR construct of tdTomato fluorescent reporter gene was delivered to iPS cells and yielded ~8% knock in efficiency 7 days post transfection (FIG. 69, FIG. 70, and FIG. 71).

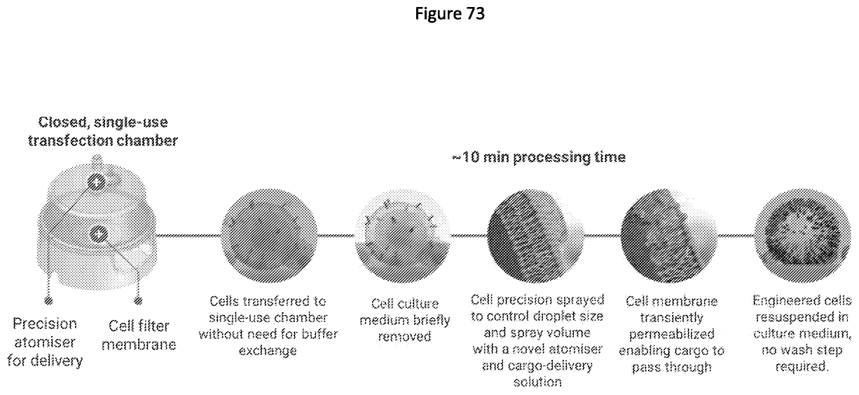

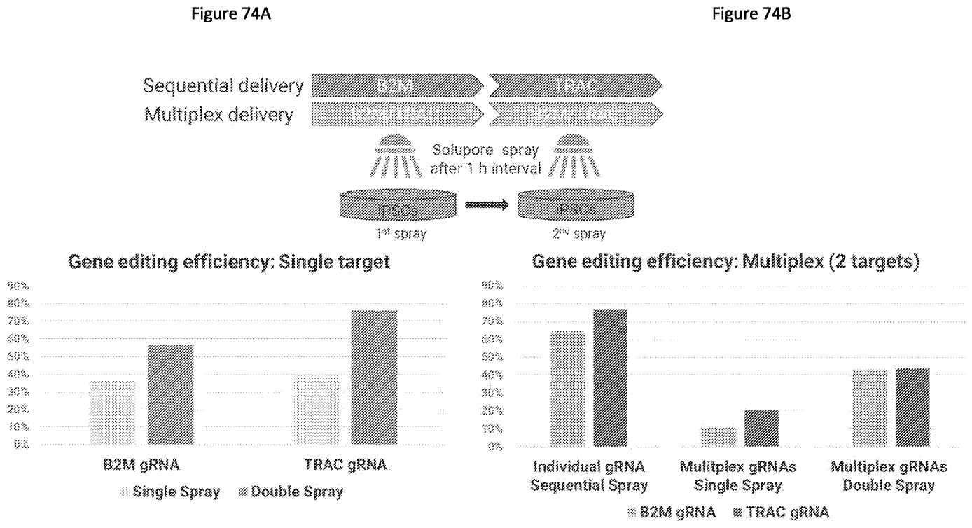

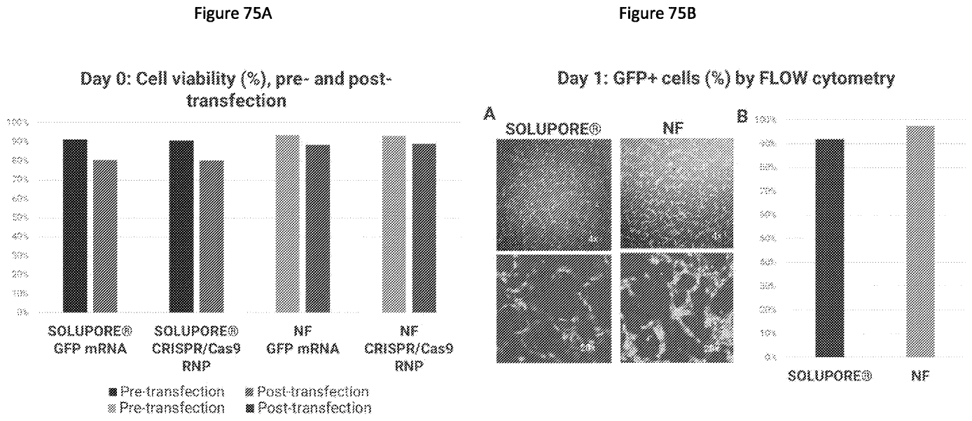

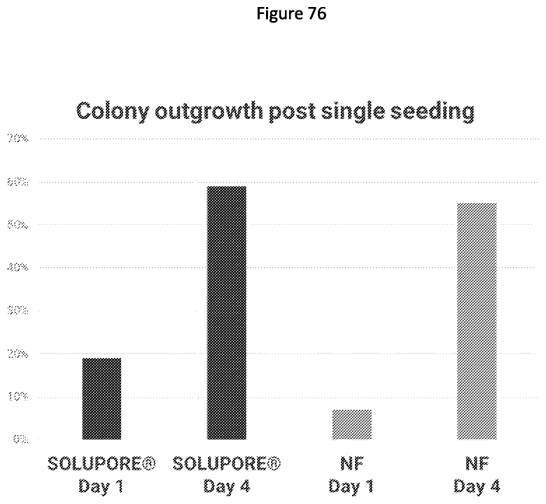

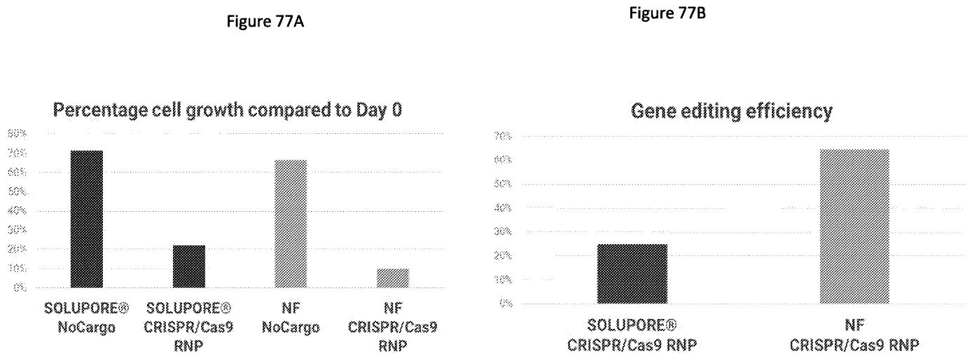

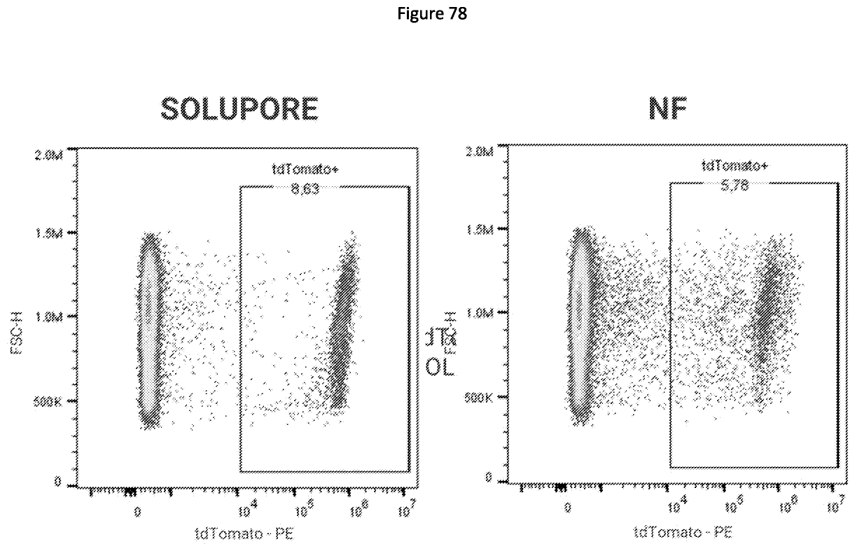

The technology efficiently produces single and multi-gene edited iPSCs. The multiplex capabilities combined with the ability to deliver sequentially, enables diverse cargo delivery options, including KO and KI editing, while maintaining cell health and reducing cell derivation workflow time (FIG. 72-78).

### Example 7: Non-Viral Reprogramming of Blood Cells to iPSCs

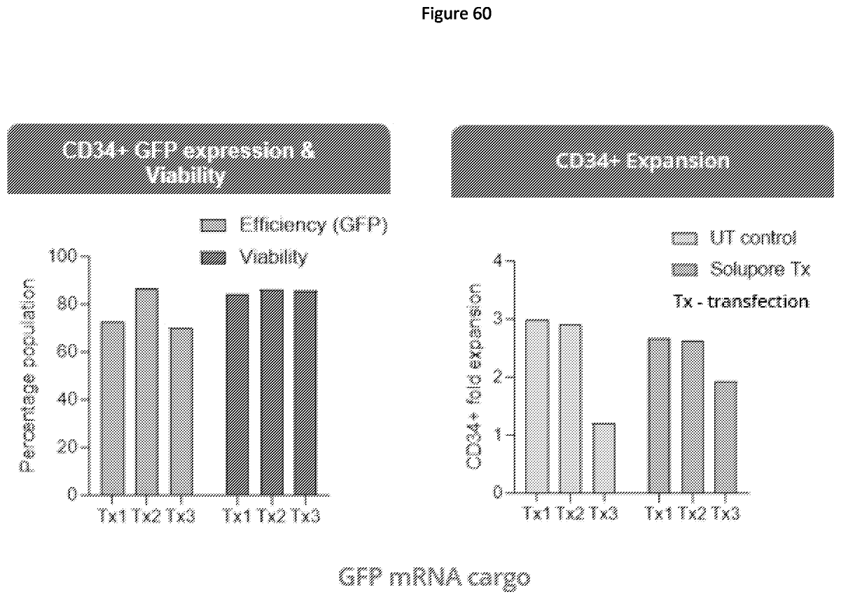

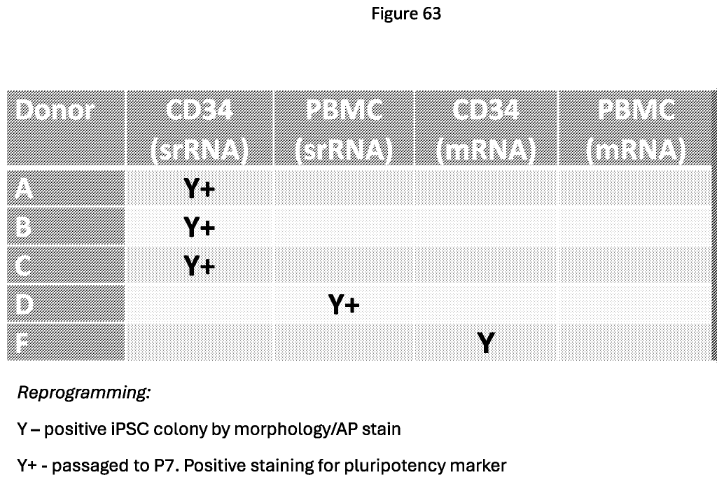

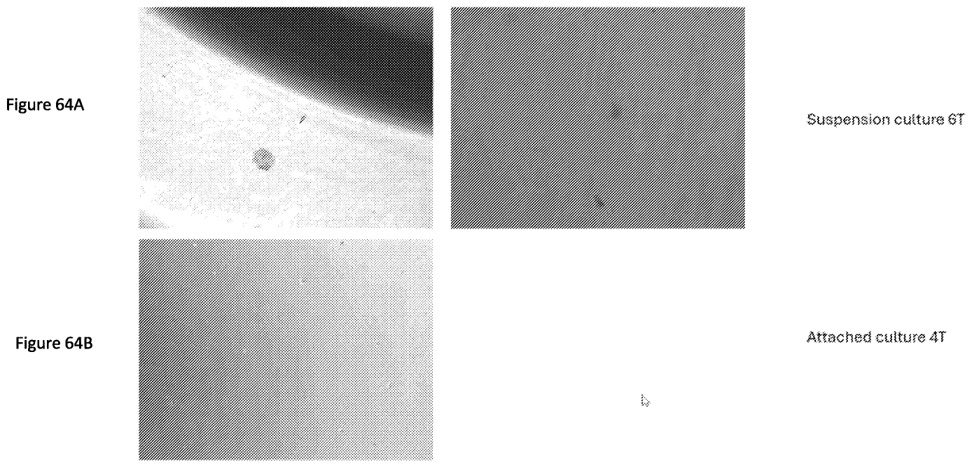

In implementations, the methods further efficiently transfected CD34+ cells with multiple RNA deliveries (FIG. 60). In implementations, reprogramming of CD34 cells to iPSCs was shown with repeated delivery of mRNA reprogramming factors using the delivery methods described herein. Positive iPSC colonies were observed from 3 out of 3 donors from CD34 cord blood starting material (immature cell populations) and 1 out of 3 donors from PBMC (mature adult cells) starting material (FIG. 63). Approximately 10 colonies (spheres) grew in suspension after 6 transfections of mRNA. Cells remained in the 3D bioreactor and the morphology was consistent (FIG. 64A).

In implementations, 6 million isolated CD34+ cells were transfected using the delivery method and device described herein. Cells were harvested from the bioreactor and again transfected with the reprogramming mRNA before being transferred back to the bioreactor. The process was repeated for a total of 6 transfections. Positive iPS colonies were observed in the bioreactor (FIG. 64A).

In implementations, reprogramming of CD34+ cells following delivery of srRNA by the method was observed. Positive iPSC colonies stained with alkaline phosphatase and reprogramming was confirmed in 3 out of 3 donors tested.

Colonies were picked from both CD34 and PBMC plates and were passaged 7 times and stained for pluripotency markers. The pluripotency of these markers was confirmed in the iPSC colonies.

In implementations, mRNA transfections of CD34+ cells were demonstrated. Successful colony formation was observed after 6 transfections of CD34+ cells with mRNA factors. Cells were kept in a 3D bioreactor between transfections and maintained therein. Six million CD34+ cells were transfected initially and only about a 40% loss was observed over the 6 transfections. As described, the confirmation of pluripotency and passaging of CD34 derived iPSC colonies from mRNA delivery was observed.

Methods for transfecting PBMCs and CD34+ cells were evaluated for delivery of srRNA and mRNA.

A single transfection of reprogramming factors in the form of self-replicating RNA (ReproRNA; Stemcell technologies) to CD34 or PBMC cells was carried out using Solupore RT. Cells were plated and cultured. Colonies were selected and passaged to P7 and stained for the pluripotency markers SSEA4 and TRA-1-60 and differentiation marker SSEA1.

Methods of transfection PBMCs and CD34 cells were evaluated for delivery of mRNA. In implementations, a CD34+ cell donor was used and eight consecutive transfections (transfection sprays) were performed.

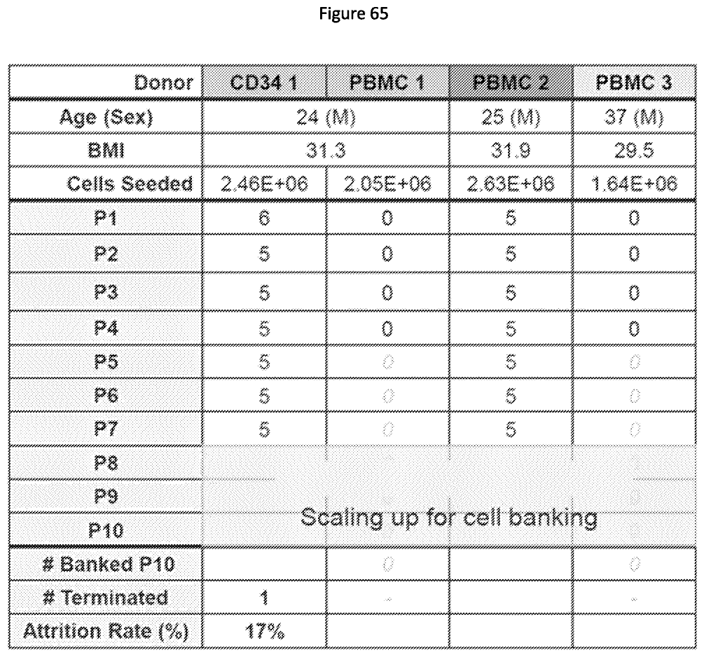

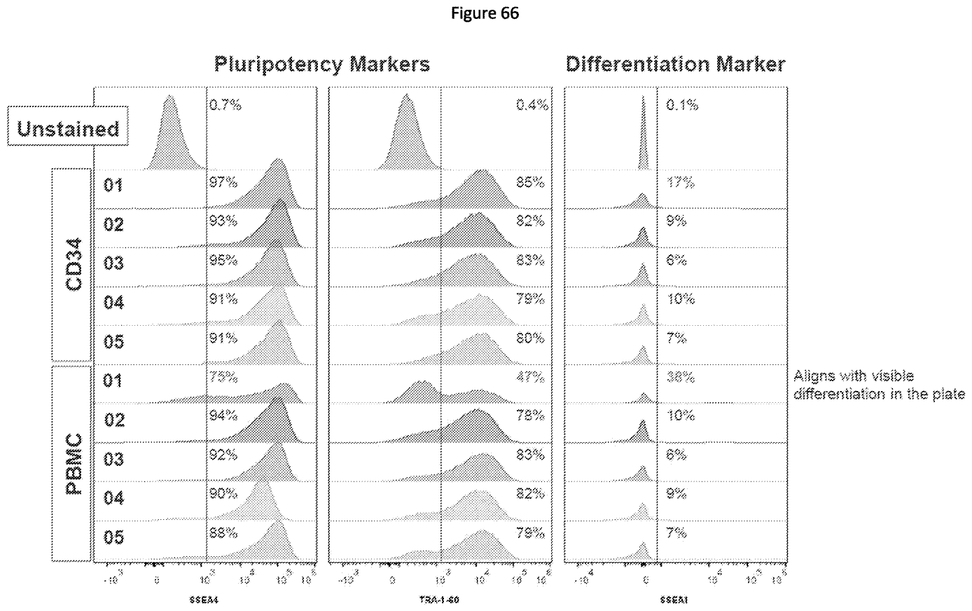

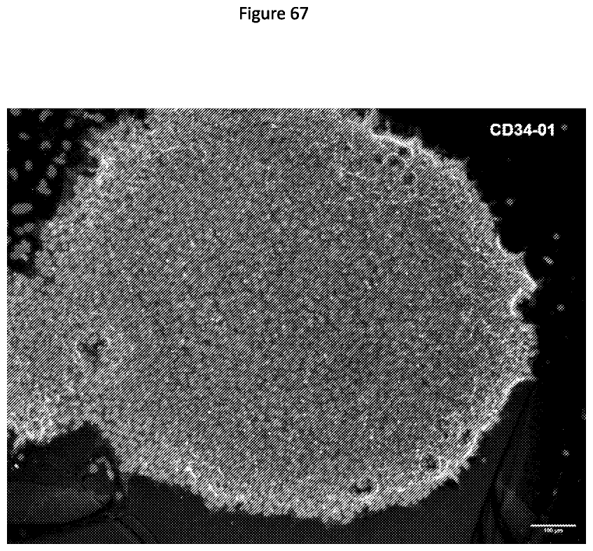

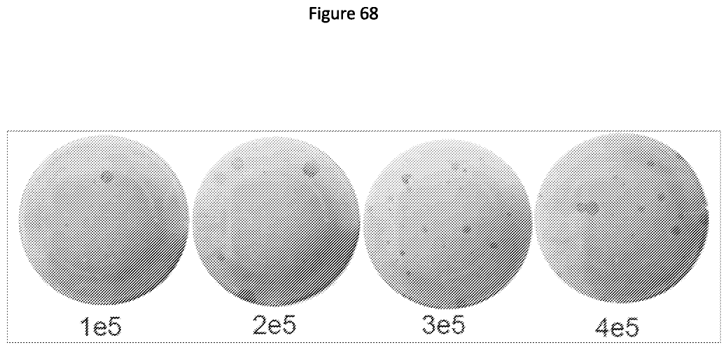

In some implementations, a PBMC donor was used and the PBMCs were kept in 3D culture (a bioreactor) prior and in between transfections. Eight consecutive transfection sprays (transfections) were performed. A live stain for SSEA-4 and TRA-1-6 was performed and when positive, the cells are passaged. iPSC clones were characterized after passage 5 (FIG. 65, FIG. 66, and FIG. 67).

In future implementations, T cells are used for non-viral reprogramming to iPSCs using the delivery method described herein.

### Other Embodiments

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A method of delivering a payload across a plasma membrane of an adherent cell, comprising,
providing a population of adherent cells,
contacting the population of cells with a volume of an aqueous solution, the aqueous solution including the payload and an alcohol at greater than 2 percent (v/v) concentration, and wherein the contacting the population of cells with the volume of the aqueous solution is performed in an environment having a temperature greater than 25 °C.

2. The method of claim 3, wherein the adherent cell comprises stem cells, wherein the stem cells comprise induced pluripotent stem cells (iPSCs), primary mesenchymal stem cells, neuronal stem cells, hematopoietic stem cells, mouse embryonic stem cells, and human embryonic stem cells.

3. The method of claim 1, wherein the environment has a temperature of about 37 °C.

4. The method of claim 1, wherein the population of cells has a temperature of about 25 °C to about 37 °C during the contacting step.

5. The method of claim 1, wherein the aqueous solution has a temperature of about 25 °C to about 37 °C during the contacting step.

6. The method of claim 1, wherein the population of cells and the aqueous solution have a temperature the same as the environment.

7. The method of claim 1, wherein the environment is an environment enclosed within an incubator.

8. The method of claim 1, wherein the stop solution has a temperature of about 25 °C, to about 37 °C during the contacting step.

9. A system comprising:
an incubator including an internal environment and configurable to maintain a temperature of the internal environment at greater than 25 °C; and a delivery platform configurable to deliver a payload to a population of cells, the delivery platform comprising:
a pod including a filter plate and an upper portion forming a well;
a housing including a pod holder configured to receive the pod;
a delivery solution applicator configured to deliver atomized delivery solution to the well;
a display; and
a controller including circuitry configured to display at least one process parameter,
wherein the delivery platform is located within the internal environment of the incubator.

10. The system of claim 9, wherein the environment has a temperature from about 37 °C to about 25 °C.

11. A system comprising:
an incubator including an internal environment and configurable to maintain a temperature of the internal environment at greater than 25 °C; and
a device for use to deliver a cargo to cells in the absence of alcohol, the device comprising:
a housing including
a base,
at least one controller including circuitry configured to control an operation of the device, and
a display;
one or more fluid circuits including at least one valve, at least one pump, a syringe, and at least one fluid detection sensor;
a chamber assembly received within an articulating frame extending from the front surface of the housing, wherein the chamber assembly is sealed from atmospheric conditions in operation and includes a filter;
at least one media container;
at least one cell culture container fluidically coupled to the chamber assembly via the one or more fluid circuits; and
at least one collection tray configured to receive media or cells, wherein the delivery platform is located within the internal environment of the incubator.

12. The system of claim 11, wherein the environment has a temperature of about 25 °C to about 37 °C.

13. A method of reprogramming a population of blood cells to a population of induced pluripotent stem cells (iPSCs), the method comprising
providing a population of blood cells,
transfecting the population of blood cells by at least contacting the population of blood cells with a volume of an aqueous solution, the aqueous solution including a nucleic acid molecule and an alcohol at a greater than 2 percent (v/v) concentration,
thereby reprogramming the population of blood cells to a population of iPSCs.

14. The method of claim 13, wherein the blood cells comprise CD34+ cells or peripheral blood mononuclear cells (PBMCs).

15. The method of claim 13, wherein the population of blood cells is at a density of about 5e4 to about 5e7.

16. The method of claim 13, wherein the nucleic acid molecule comprises a ribonucleic acid (RNA) molecule.

17. The method of claim 13, wherein the RNA molecule comprises self-replicating RNA (srRNA), messenger RNA (mRNA), siRNA, or RNAi.

18. The method of claim 13, wherein the population of blood cells is transfected between 2 and 10 times, or wherein the population of blood cells is transfected 5 times, 6 times, 7 times, 8 times, 9 times, or 10 times.

19. The method of claim 13, further comprising performing multiple transfections on the population of blood cells.

20. The method of claim 13, wherein the aqueous solution comprises an isotonic aqueous solution.

21. The method of claim 13, wherein the nucleic acid molecule encodes a gene-editing composition.

22. A population of blood-cell derived induced pluripotent stem cells (iPSCs),
wherein the population is generated without the use of a virus or viral vector, and
wherein the blood cells are contacted with a volume of an aqueous solution, the aqueous solution including a nucleic acid molecule and an alcohol at a greater than 2 percent (v/v) concentration.
